SEQUENCE LISTING (243 sequences)

SEQ ID NO: 1 - protein, 10 aa, synthetic construct
  REGION 1..10: note = Synthetic: Heavy Chain - > 491-1C6 (LJI_0491-30).1 , CRDH1
GGSISRSSHY

SEQ ID NO: 2 - protein, 7 aa, synthetic construct
  REGION 1..7: note = Synthetic: Heavy Chain - > 491-1C6 (LJI_0491-30).2, CRDH2
IYYSGTT

SEQ ID NO: 3 - protein, 15 aa, synthetic construct
  REGION 1..15: note = Synthetic: Heavy Chain - > 491-1C6 (LJI_0491-30).3, CRDH3
ARRRAGAALPGVEDY

SEQ ID NO: 4 - protein, 122 aa, synthetic construct
  REGION 1..122: note = Synthetic: Heavy Chain - > 491-1C6 (LJI_0491-30).4, AA
QVQLVQSGPGLVKPSETLSLTCTVSGGSISRSSHYWGWIRQPPGKGLEWIGSIYYSGTTYYNPSLKSRVTISVDTSKNQFSLKLNSVTAADTAVYFCARRRAGAALPGVEDYWGQGTLVTVS

SEQ ID NO: 5 - DNA, 366 bp, synthetic construct
  misc_feature 1..366: note = Synthetic: Heavy Chain - > 491-1C6 (LJI_0491-30).5, Nt
  source 1..366: mol_type = other DNA
caggtccagctggtacagtctggcccaggactggtgaagccttcggagaccctgtccctcacctgcactgtctctggtggctccatcagcagaagtagtcactactggggctggatccgccagcccccagggaaggggctggagtggattgggagtatctattatagtgggaccacctactacaatccgtccctcaagagtcgagtcaccatatccgtagacacgtccaagaaccagttctccctgaagctgaactctgtgaccgccgcagacacggctgtgtatttctgtgcgagacgaagggctggagcagctcttccgggggtggaggactactggggccagggaaccctggtcaccgtctcc

SEQ ID NO: 6 - protein, 8 aa, synthetic construct
  REGION 1..8: note = Synthetic: Light Chain - > 491-1C6 (LJI_0491-30).1, CDRL1
SGSIASNY

SEQ ID NO: 7 - skipped

SEQ ID NO: 8 - protein, 10 aa, synthetic construct
  REGION 1..10: note = Synthetic: Light Chain - > 491-1C6 (LJI_0491-30).3, CDRL3
QSYDSSSQEV

SEQ ID NO: 9 - protein, 111 aa, synthetic construct
  REGION 1..111: note = Synthetic: Light Chain - > 491-1C6 (LJI_0491-30).4, AA
RPVLTQPHSVSESPGKTVTISCTRSSGSIASNYVQWFQLRPGSAPTTVIYEDNQRLSGVPDRFSGSIDSSSNSASLTISGLKTEDEAEYYCQSYDSSSQEVFGGGTKLTVL

SEQ ID NO: 10 - DNA, 333 bp, synthetic construct
  misc_feature 1..333: note = Synthetic: Light Chain - > 491-1C6 (LJI_0491-30).5, Nt
  source 1..333: mol_type = other DNA
cggcccgtgctgactcagccccactctgtgtcggagtctccggggaagacggtaaccatctcctgcacccgcagcagtggcagcattgccagcaactatgtgcagtggttccagctgcgcccgggcagtgcccccaccactgtgatctatgaggataaccaaagactctctggggtccctgatcggttctctggctccatcgacagctcctccaactctgcctccctcaccatctctggactgaagactgaggacgaggctgagtactactgtcagtcttatgatagcagcagtcaggaggtattcggcggagggaccaagctgaccgtccta

SEQ ID NO: 11 - protein, 10 aa, synthetic construct
  REGION 1..10: note = Synthetic: Heavy Chain - > 491-1C6 (LJI_0491-30).2, CRDH1
GGSISRSSHY

SEQ ID NO: 12 - protein, 7 aa, synthetic construct
  REGION 1..7: note = Synthetic: Heavy Chain - > 491-1C6 (LJI_0491-30).3, CRDH2
IYYSGTT

SEQ ID NO: 13 - protein, 15 aa, synthetic construct
  REGION 1..15: note = Synthetic: Heavy Chain - > 491-1C6 (LJI_0491-30).4, CRDH3
ARRRAGAALPGVEDY

SEQ ID NO: 14 - protein, 122 aa, synthetic construct
  REGION 1..122: note = Synthetic: Heavy Chain - > 491-1C6 (LJI_0491-30).5, AA
QVQLQQLGPGLVKPSETLSLTCTVFGGSISRSSHYWGWIRQPPGKGLEWIGSIYYSGTTYYNPSLKSRVTISVDTSKNQFSLKLNSVTAADTAVYFCARRRAGAALPGVEDYWGQGTLVTVS

SEQ ID NO: 15 - DNA, 366 bp, synthetic construct
  misc_feature 1..366: note = Synthetic: Heavy Chain - > 491-1C6 (LJI_0491-30).6, Nt
  source 1..366: mol_type = other DNA
caggtgcagctgcagcagttgggcccaggactggtgaagccttcggagaccctgtccctcacctgcactgtttttggtggctccatcagcagaagtagtcactactggggctggatccgccagcccccagggaaggggctggagtggattgggagtatttattatagtgggaccacctactacaatccgtccctcaagagtcgagtcaccatatccgtagacacgtccaagaaccagttttccctgaagctgaactctgtgaccgccgcagacacggctgtgtatttctgtgcgagacgaagggctggagcagctcttccgggggtggaggactactggggccagggaaccctggtcaccgtctcc

SEQ ID NO: 16 - protein, 8 aa, synthetic construct
  REGION 1..8: note = Synthetic: Light Chain - > 491-1C6 (LJI_0491-30).2, CDRL1
SGSIASNY

SEQ ID NO: 17 - skipped

SEQ ID NO: 18 - protein, 10 aa, synthetic construct
  REGION 1..10: note = Synthetic: Light Chain - > 491-1C6 (LJI_0491-30).4, CDRL3
QSYDSSSQEV

SEQ ID NO: 19 - protein, 111 aa, synthetic construct
  REGION 1..111: note = Synthetic: Light Chain - > 491-1C6 (LJI_0491-30).5, AA
NFMLTQPHSVSESPGKTVTISCTRSSGSIASNYVQWYQLRPGSAPTTVIYEDNQRPSGVPDRFFGSIDSSSNSASLTISGLKTEDEAEYYCQSYDSSSQEVFGGGTKLTVL

SEQ ID NO: 20 - DNA, 333 bp, synthetic construct
  misc_feature 1..333: note = Synthetic: Light Chain - > 491-1C6 (LJI_0491-30).6, Nt
  source 1..333: mol_type = other DNA
aattttatgttgactcagccgcactctgtgtcggagtctccggggaagacggtaaccatctcctgcacccgcagcagtggcagcattgccagcaattatgtgcagtggtaccagctgcgcccgggcagtgcccccaccactgtgatctatgaggataaccaaagaccctctggggtccctgatcggttctttggctccatcgacagctcctccaactctgcctccctcaccatctctggactgaagactgaggacgaggctgagtactactgtcagtcttatgatagcagcagtcaggaggtattcggcggagggaccaagctgaccgtccta

SEQ ID NO: 21 - protein, 8 aa, synthetic construct
  REGION 1..8: note = Synthetic: Heavy Chain - > 491-1H2b (LJI_0491-86b), CRDH1
GFTFDDYA

SEQ ID NO: 22 - protein, 8 aa, synthetic construct
  REGION 1..8: note = Synthetic: Heavy Chain - > 491-1H2b (LJI_0491-86b), CRDH2
ITWNSGTI

SEQ ID NO: 23 - protein, 19 aa, synthetic construct
  REGION 1..19: note = Synthetic: Heavy Chain - > 491-1H2b (LJI_0491-86b), CRDH3
AKDSGRKLLWGREDYYMGV

SEQ ID NO: 24 - protein, 125 aa, synthetic construct
  REGION 1..125: note = Synthetic: Heavy Chain - > 491-1H2b (LJI_0491-86b), AA
QVQLQQSGGGLVQPGRSLRLSCAASGFTFDDYAMHWVRQAPGKGLEWVSGITWNSGTIGYADSVKGRFTISRDNAKNSLYLQMRSLRAEDTALYYCAKDSGRKLLWGREDYYMGVWGKGTTVTVS

SEQ ID NO: 25 - DNA, 375 bp, synthetic construct
  misc_feature 1..375: note = Synthetic: Heavy Chain - > 491-1H2b (LJI_0491-86b), Nt
  source 1..375: mol_type = other DNA
caggtacagctgcagcagtcagggggaggcttggtacagcctggcaggtccctgagactctcctgtgcagcctctggattcacctttgatgattatgccatgcactgggtccggcaagctccagggaagggcctggagtgggtctcaggtattacttggaatagtggtaccataggctatgcggactctgtgaagggccgattcaccatctccagagacaacgccaagaactccctctatctgcaaatgagaagtcttagagctgaggacacggccttgtattactgtgcaaaagattccgggcggaaactgctttgggggcgtgaggactactacatgggcgtctggggcaaagggaccacggtcaccgtctcc

SEQ ID NO: 26 - protein, 8 aa, synthetic construct
  REGION 1..8: note = Synthetic: Heavy Chain - > 491-1H2e (LJI_0491-86e).1, CRDH1
GFTFDDYA

SEQ ID NO: 27 - protein, 8 aa, synthetic construct
  REGION 1..8: note = Synthetic: Heavy Chain - > 491-1H2e (LJI_0491-86e).1, CRDH2
ITWNSGTI

SEQ ID NO: 28 - protein, 19 aa, synthetic construct
  REGION 1..19: note = Synthetic: Heavy Chain - > 491-1H2e (LJI_0491-86e).1, CRDH3
AKDSGRKQLWGREDYYMGV

SEQ ID NO: 29 - protein, 125 aa, synthetic construct
  REGION 1..125: note = Synthetic: Heavy Chain - > 491-1H2e (LJI_0491-86e).1, AA
QVQLVQSGGGLVQPGRSLRLSCAASGFTFDDYAMHWVRQAPGKGLEWVSGITWNSGTIGYADTVKGRFTISRDNAKNSLYLQMRSLRAEDTALYYCAKDSGRKQLWGREDYYMGVWGKGTTVTVS

SEQ ID NO: 30 - DNA, 375 bp, synthetic construct
  misc_feature 1..375: note = Synthetic: Heavy Chain - > 491-1H2e (LJI_0491-86e).1, Nt
  source 1..375: mol_type = other DNA
caggtccagctggtacagtctggtggaggcttggtacagcctggcaggtccctgagactctcctgtgcagcctctggattcacctttgatgattatgccatgcactgggtccggcaagctccagggaagggcctggagtgggtctcaggtattacttggaatagtggtaccataggctatgcggacactgtgaagggccgattcaccatctccagagacaacgccaagaactccctctatctgcaaatgagaagtcttagagctgaggacacggccttgtattactgtgcaaaagattccgggcggaaacagctttgggggcgtgaggactactacatgggcgtctggggcaaagggaccacggtcaccgtctcc

SEQ ID NO: 31 - protein, 8 aa, synthetic construct
  REGION 1..8: note = Synthetic: Heavy Chain - > 491-1H2e (LJI_0491-86e).2, CRDH1
GFTFDDYA

SEQ ID NO: 32 - protein, 8 aa, synthetic construct
  REGION 1..8: note = Synthetic: Heavy Chain - > 491-1H2e (LJI_0491-86e).2, CRDH2
ITWNSGTI

SEQ ID NO: 33 - protein, 19 aa, synthetic construct
  REGION 1..19: note = Synthetic: Heavy Chain - > 491-1H2e (LJI_0491-86e).2, CRDH3
AKDSGRKQLWGREDYYMGV

SEQ ID NO: 34 - protein, 125 aa, synthetic construct
  REGION 1..125: note = Synthetic: Heavy Chain - > 491-1H2e (LJI_0491-86e).2, AA
EVQLVEFGGGLVQPGRSLRLSCAASGFTFDDYAMHWVRQAPGKGLEWVSGITWNSGTIGYADSVKGRFTISRDNAKNSLYLQMRSLRAEDTALYYCAKDSGRKQLWGREDYYMGVWGKGTTVTVS

SEQ ID NO: 35 - DNA, 375 bp, synthetic construct
  misc_feature 1..375: note = Synthetic: Heavy Chain - > 491-1H2e (LJI_0491-86e).2, Nt
  source 1..375: mol_type = other DNA
gaagtgcagctggtggagtctgggggaggcttggtacagcctggcaggtccctgagactttcctgtgcagcctttggattcacctttgatgattatgccatgcactgggtccggcaagctccagggaagggcctggagtgggtttcaggtattacttggaatagtggtaccataggctatgcggactctgtgaagggccgattcaccatctccagagacaacgccaagaactccctctatctgcaaatgagaagtcttagagctgaggacacggccttgtattactgtgcaaaagattccgggcggaaacagctttgggggcgtgaggactactacatgggcgtctggggcaaagggaccacggtcaccgtctcc

SEQ ID NO: 36 - protein, 6 aa, synthetic construct
  REGION 1..6: note = Synthetic: Light Chain - > 491-1H2 (LJI_0491-86), CDRL1
NIGSKS

SEQ ID NO: 37 - skipped

SEQ ID NO: 38 - protein, 11 aa, synthetic construct
  REGION 1..11: note = Synthetic: Light Chain - > 491-1H2 (LJI_0491-86), CDRL3
QVWDSSSDHVV

SEQ ID NO: 39 - protein, 108 aa, synthetic construct
  REGION 1..108: note = Synthetic: Light Chain - > 491-1H2 (LJI_0491-86), AA
SYELTQPPSVSVAPGKTARITCGGSNIGSKSVHWYQQKPGQAPVLVIYYDSDRPSGIPERFSGSNSGNTATLTISRVEAGDEADYYCQVWDSSSDHVVFGGGTKLTVL

SEQ ID NO: 40 - DNA, 324 bp, synthetic construct
  misc_feature 1..324: note = Synthetic: Light Chain - > 491-1H2 (LJI_0491-86), Nt
  source 1..324: mol_type = other DNA
tcctatgagctgactcagccaccctctgtgtcagtggccccaggaaagacggccaggattacttgtgggggaagcaacattgggagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagcagtgatcatgtggtattcggcggagggaccaagctgaccgtccta

SEQ ID NO: 41 - protein, 8 aa, synthetic construct
  REGION 1..8: note = Synthetic: Heavy Chain - > 491-2A1 (LJI_0491-97).1, CRDH1
GYRFTRHW

SEQ ID NO: 42 - protein, 8 aa, synthetic construct
  REGION 1..8: note = Synthetic: Heavy Chain - > 491-2A1 (LJI_0491-97).1, CRDH2
IYPGDSDT

SEQ ID NO: 43 - protein, 21 aa, synthetic construct
  REGION 1..21: note = Synthetic: Heavy Chain - > 491-2A1 (LJI_0491-97).1, CRDH3
ARHVYCSSSTCYTTEGGWFDA

SEQ ID NO: 44 - protein, 127 aa, synthetic construct
  REGION 1..127: note = Synthetic: Heavy Chain - > 491-2A1 (LJI_0491-97).1, AA
QVQLVESGAEVKKPGESLKISCKGSGYRFTRHWIGWVRQMPGKGLEWMGIIYPGDSDTRYSPSFQGQVTISADKSIGTAYVQWSSLKASDTAMYYCARHVYCSSSTCYTTEGGWFDAWGQGTLVTVS

SEQ ID NO: 45 - DNA, 381 bp, synthetic construct
  misc_feature 1..381: note = Synthetic: Heavy Chain - > 491-2A1 (LJI_0491-97).1, Nt
  source 1..381: mol_type = other DNA
caggtacagctcgtggagtctggagcagaggtgaaaaagccgggggagtctctgaagatctcctgtaagggttctggatacaggtttaccagacactggatcggctgggtgcgccagatgcccgggaaaggcctggagtggatggggatcatctatcctggtgactctgataccagatacagcccgtccttccaaggccaggtcaccatctcagccgacaagtccatcggcaccgcatatgtgcagtggagcagcctgaaggcctcggacaccgccatgtattattgtgcgagacatgtatattgtagtagtagcacctgctacacgaccgagggggggtggttcgacgcctggggccagggaaccctggtcaccgtctcc

SEQ ID NO: 46 - protein, 8 aa, synthetic construct
  REGION 1..8: note = Synthetic: Heavy Chain - > 491-2A1 (LJI_0491-97).2, CRDH1
GYRFTRHW

SEQ ID NO: 47 - protein, 8 aa, synthetic construct
  REGION 1..8: note = Synthetic: Heavy Chain - > 491-2A1 (LJI_0491-97).2, CRDH2
IYPGDSDT

SEQ ID NO: 48 - protein, 21 aa, synthetic construct
  REGION 1..21: note = Synthetic: Heavy Chain - > 491-2A1 (LJI_0491-97).2, CRDH3
ARHVYCSSSTCYTTEGGWFDA

SEQ ID NO: 49 - protein, 127 aa, synthetic construct
  REGION 1..127: note = Synthetic: Heavy Chain - > 491-2A1 (LJI_0491-97).2, AA
EVQLVQSGAEVKKPGESLKISCKGSGYRFTRHWIGWVRQMPGKGLEWMGIIYPGDSDTRYSPSFQGQVTISADKSIGTAYVQWSSLKASDTAMYYCARHVYCSSSTCYTTEGGWFDAWGQGTLVTVS

SEQ ID NO: 50 - DNA, 381 bp, synthetic construct
  misc_feature 1..381: note = Synthetic: Heavy Chain - > 491-2A1 (LJI_0491-97).2, Nt
  source 1..381: mol_type = other DNA
gaggtgcagctggtgcagtctggagcagaggtgaaaaagccgggggagtctctgaagatctcctgtaagggttctggatacaggtttaccagacactggatcggctgggtgcgccagatgcccgggaaaggcctggagtggatggggatcatctatcctggtgactctgataccagatacagcccgtccttccaaggccaggtcaccatctcagccgacaagtccatcggcaccgcatatgtgcagtggagcagcctgaaggcctcggacaccgccatgtattattgtgcgagacatgtatattgtagtagtagcacctgctacacgaccgagggggggtggttcgacgcctggggccagggaaccctggtcaccgtctcc

SEQ ID NO: 51 - protein, 8 aa, synthetic construct
  REGION 1..8: note = Synthetic: Light Chain - > 491-2A1 (LJI_0491-97), CDRL1
SSNIGSNT

SEQ ID NO: 52 - skipped

SEQ ID NO: 53 - protein, 11 aa, synthetic construct
  REGION 1..11: note = Synthetic: Light Chain - > 491-2A1 (LJI_0491-97), CDRL3
AAWDDSLNGVV

SEQ ID NO: 54 - protein, 110 aa, synthetic construct
  REGION 1..110: note = Synthetic: Light Chain - > 491-2A1 (LJI_0491-97), AA
QSVLTQPPSASGTPGQRVTISCSGSSSNIGSNTVNWYQQLPGTAPKLLIYSNNQRPSGVPDRFSDSKSGTSASLAISGLQSEDEADYYCAAWDDSLNGVVFGGGTKLTVL

SEQ ID NO: 55 - DNA, 330 bp, synthetic construct
  misc_feature 1..330: note = Synthetic: Light Chain - > 491-2A1 (LJI_0491-97), Nt
  source 1..330: mol_type = other DNA
cagtctgttctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctgactccaagtctggcacctcagcctccctggccatcagtgggctccagtctgaggatgaggctgattattactgtgcagcctgggatgacagcctgaatggtgtggtattcggcggagggaccaagcttaccgtccta

SEQ ID NO: 56 - protein, 8 aa, synthetic construct
  REGION 1..8: note = Synthetic: Heavy Chain - > 491-2A4 (LJI_0491-100).1, CRDH1
GITVSKNY

SEQ ID NO: 57 - protein, 7 aa, synthetic construct
  REGION 1..7: note = Synthetic: Heavy Chain - > 491-2A4 (LJI_0491-100).1, CRDH2
IYSGGST

SEQ ID NO: 58 - protein, 10 aa, synthetic construct
  REGION 1..10: note = Synthetic: Heavy Chain - > 491-2A4 (LJI_0491-100).1, CRDH3
ARDLGDYFDY

SEQ ID NO: 59 - protein, 115 aa, synthetic construct
  REGION 1..115: note = Synthetic: Heavy Chain - > 491-2A4 (LJI_0491-100).1, AA
QVQLVQSGGGLIQPGGSLRLSCAASGITVSKNYMNWVRQAPGKGLEWVSVIYSGGSTFYADSVKGRFTISRDNSKNTLYLQMNSLRAEDTAVYYCARDLGDYFDYWGQGTLVTVS

SEQ ID NO: 60 - DNA, 345 bp, synthetic construct
  misc_feature 1..345: note = Synthetic: Heavy Chain - > 491-2A4 (LJI_0491-100).1, Nt
  source 1..345: mol_type = other DNA
caggtccagctggtacagtctggaggaggcttgatccagcctggggggtccctgagactctcctgtgcagcctctgggatcaccgtcagtaaaaactacatgaactgggtccgccaggctccagggaaggggctggagtgggtctcagttatttatagcggtggtagcacattctacgcagactccgtgaagggccgattcaccatctccagagacaattccaagaacacgctgtatctgcaaatgaacagcctgagagccgaggacacggccgtgtattactgtgcgagagatctcggtgactactttgactactggggccagggaaccctggtcaccgtctcc

SEQ ID NO: 61 - protein, 6 aa, synthetic construct
  REGION 1..6: note = Synthetic: Light Chain - > 491-2A4 (LJI_0491-100).1, CDRL1
QGISSY

SEQ ID NO: 62 - skipped

SEQ ID NO: 63 - protein, 10 aa, synthetic construct
  REGION 1..10: note = Synthetic: Light Chain - > 491-2A4 (LJI_0491-100).1, CDRL3
QQLNSYPPNT

SEQ ID NO: 64 - protein, 108 aa, synthetic construct
  REGION 1..108: note = Synthetic: Light Chain - > 491-2A4 (LJI_0491-100).1, AA
DIVMTQTPSFLSASVGDRVTITCRASQGISSYLAWYQQKPGKAPKLLIYAASTLQSGVPSRFSGSGSGTEFTLTISSLQPEDFATYYCQQLNSYPPNTFGQGTKLEIK

SEQ ID NO: 65 - DNA, 324 bp, synthetic construct
  misc_feature 1..324: note = Synthetic: Light Chain - > 491-2A4 (LJI_0491-100).1, Nt
  source 1..324: mol_type = other DNA
gatattgtgatgacccagactccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctccgaacacttttggccaggggaccaagctggagatcaaa

SEQ ID NO: 66 - protein, 8 aa, synthetic construct
  REGION 1..8: note = Synthetic: Heavy Chain - > 491-2A4 (LJI_0491-100).2, CRDH1
GITVSKNY

SEQ ID NO: 67 - protein, 7 aa, synthetic construct
  REGION 1..7: note = Synthetic: Heavy Chain - > 491-2A4 (LJI_0491-100).2, CRDH2
IYSGGST

SEQ ID NO: 68 - protein, 10 aa, synthetic construct
  REGION 1..10: note = Synthetic: Heavy Chain - > 491-2A4 (LJI_0491-100).2, CRDH3
ARDLGDYFDY

SEQ ID NO: 69 - protein, 115 aa, synthetic construct
  REGION 1..115: note = Synthetic: Heavy Chain - > 491-2A4 (LJI_0491-100).2, AA
EVQLVESGGGLIQPGGSLRLSCAASGITVSKNYMNWVRQAPGKGLEWVSVIYSGGSTFYADSVKGRFTISRDNSKNTLYLQMNSLRAEDTAVYYCARDLGDYFDYWGQGTLVTVS

SEQ ID NO: 70 - DNA, 345 bp, synthetic construct
  misc_feature 1..345: note = Synthetic: Heavy Chain - > 491-2A4 (LJI_0491-100).2, Nt
  source 1..345: mol_type = other DNA
gaggtgcagctggtggagtctggaggaggcttgatccagcctggggggtccctgagactctcctgtgcagcctctgggatcaccgtcagtaaaaactacatgaactgggtccgccaggctccagggaaggggctggagtgggtctcagttatttatagcggtggtagcacattctacgcagactccgtgaagggccgattcaccatctccagagacaattccaagaacacgctgtatctgcaaatgaacagcctgagagccgaggacacggccgtgtattactgtgcgagagatctcggtgactactttgactactggggccagggaaccctggtcaccgtctcc

SEQ ID NO: 71 - protein, 6 aa, synthetic construct
  REGION 1..6: note = Synthetic: Light Chain - > 491-2A4 (LJI_0491-100).2, CDRL1
QGISSY

SEQ ID NO: 72 - skipped

SEQ ID NO: 73 - protein, 10 aa, synthetic construct
  REGION 1..10: note = Synthetic: Light Chain - > 491-2A4 (LJI_0491-100).2, CDRL3
QQLNSYPPNT

SEQ ID NO: 74 - protein, 108 aa, synthetic construct
  REGION 1..108: note = Synthetic: Light Chain - > 491-2A4 (LJI_0491-100).2, AA
DIQLTQSPSFLSASVGDRVTITCRASQGISSYLAWYQQKPGKAPKLLIYAASTLQSGVPSRFSGSGSGTEFTLTISSLQPEDFATYYCQQLNSYPPNTFGQGTKLEIK

SEQ ID NO: 75 - DNA, 324 bp, synthetic construct
  misc_feature 1..324: note = Synthetic: Light Chain - > 491-2A4 (LJI_0491-100).2, Nt
  source 1..324: mol_type = other DNA
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctccgaacacttttggccaggggaccaagctggagatcaaa

SEQ ID NO: 76 - protein, 8 aa, synthetic construct
  REGION 1..8: note = Synthetic: Heavy Chain - > 491-2A5 (LJI_0491-101).1, CRDH1
GYTFTSYD

SEQ ID NO: 77 - protein, 8 aa, synthetic construct
  REGION 1..8: note = Synthetic: Heavy Chain - > 491-2A5 (LJI_0491-101).1, CRDH2
MNPNSGNT

SEQ ID NO: 78 - protein, 23 aa, synthetic construct
  REGION 1..23: note = Synthetic: Heavy Chain - > 491-2A5 (LJI_0491-101).1, CRDH3
ARGRYFDWLKGIEDYEEDRVGNY

SEQ ID NO: 79 - protein, 129 aa, synthetic construct
  REGION 1..129: note = Synthetic: Heavy Chain - > 491-2A5 (LJI_0491-101).1, AA
EVQLLESGAEVKKPGASVKVSCKASGYTFTSYDINWVRQATGQGLEWMGWMNPNSGNTGYAQKFQGRVTMTRNTSISTAYMELSSLRSEDTAVYYCARGRYFDWLKGIEDYEEDRVGNYWGQGTLVTVS

SEQ ID NO: 80 - DNA, 387 bp, synthetic construct
  misc_feature 1..387: note = Synthetic: Heavy Chain - > 491-2A5 (LJI_0491-101).1, Nt.
  source 1..387: mol_type = other DNA
gaggtgcagctgttggagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagaggcagatattttgactggttaaagggaatagaggactacgaagaagatagggtggggaactactggggccagggaaccctggtcaccgtctcc

SEQ ID NO: 81 - protein, 6 aa, synthetic construct
  REGION 1..6: note = Synthetic: Light Chain - > 491-2A5 (LJI_0491-101).1, CDRL1
QSVSSY

SEQ ID NO: 82 - skipped

SEQ ID NO: 83 - protein, 10 aa, synthetic construct
  REGION 1..10: note = Synthetic: Light Chain - > 491-2A5 (LJI_0491-101).1, CDRL3
QQRSNWPPLT

SEQ ID NO: 84 - protein, 108 aa, synthetic construct
  REGION 1..108: note = Synthetic: Light Chain - > 491-2A5 (LJI_0491-101).1, AA
DIVMTQSPSTLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 85 - DNA, 324 bp, synthetic construct
  misc_feature 1..324: note = Synthetic: Light Chain - > 491-2A5 (LJI_0491-101).1, Nt
  source 1..324: mol_type = other DNA
gacatcgtgatgacccagtctccatccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgctcactttcggcggagggaccaaggtggagatcaaa

SEQ ID NO: 86 - protein, 8 aa, synthetic construct
  REGION 1..8: note = Synthetic: Heavy Chain - > 491-2A5 (LJI_0491-101).2, CRDH1
GYTFTSYD

SEQ ID NO: 87 - protein, 8 aa, synthetic construct
  REGION 1..8: note = Synthetic: Heavy Chain - > 491-2A5 (LJI_0491-101).2, CRDH2
MNPNSGNT

SEQ ID NO: 88 - protein, 23 aa, synthetic construct
  REGION 1..23: note = Synthetic: Heavy Chain - > 491-2A5 (LJI_0491-101).2, CRDH3
ARGRYFDWLKGIEDYEEDRVGNY

SEQ ID NO: 89 - protein, 129 aa, synthetic construct
  REGION 1..129: note = Synthetic: Heavy Chain - > 491-2A5 (LJI_0491-101).2, AA
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARGRYFDWLKGIEDYEEDRVGNYWGQGTLVTVS

SEQ ID NO: 90 - DNA, 387 bp, synthetic construct
  misc_feature 1..387: note = Synthetic: Heavy Chain - > 491-2A5 (LJI_0491-101).2, Nt
  source 1..387: mol_type = other DNA
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagaggcagatattttgactggttaaagggaatagaggactacgaagaagatagggtggggaactactggggccagggaaccctggtcaccgtctcc

SEQ ID NO: 91 - protein, 6 aa, synthetic construct
  REGION 1..6: note = Synthetic: Light Chain - > 491-2A5 (LJI_0491-101).2, CDRL1
QSVSSY

SEQ ID NO: 92 - skipped

SEQ ID NO: 93 - protein, 10 aa, synthetic construct
  REGION 1..10: note = Synthetic: Light Chain - > 491-2A5 (LJI_0491-101).2, CDRL3
QQRSNWPPLT

SEQ ID NO: 94 - protein, 108 aa, synthetic construct [sequence omitted]

SEQ ID NO: 95 - DNA, 324 bp, synthetic construct
  misc_feature 1..324: note = Synthetic: Light Chain - > 491-2A5 (LJI_0491-101).2, Nt
  source 1..324: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgctcactttcggcggagggaccaaggtggagatcaaa

SEQ ID NO: 96 - protein, 8 aa, synthetic construct
  REGION 1..8: note = Synthetic: Heavy Chain - > 491-2A10 (LJI_0491-106).1, CRDH1
EFIVSSNY

SEQ ID NO: 97 - protein, 7 aa, synthetic construct
  REGION 1..7: note = Synthetic: Heavy Chain - > 491-2A10 (LJI_0491-106).1, CRDH2
LYSGGTT

SEQ ID NO: 98 - protein, 11 aa, synthetic construct
  REGION 1..11: note = Synthetic: Heavy Chain - > 491-2A10 (LJI_0491-106).1, CRDH3
ARDRGPWLHDY

SEQ ID NO: 99 - protein, 116 aa, synthetic construct
  REGION 1..116: note = Synthetic: Heavy Chain - > 491-2A10 (LJI_0491-106).1, AA
QVQLVESGGGLIQPGGSLRLSCAASEFIVSSNYMSWVRQAPGKGLEWVSLLYSGGTTYYADSVKGRFTISRDNSKNTLFLQMNSLRAEDTAMYYCARDRGPWLHDYWGQGTLVTVS

SEQ ID NO: 100 - DNA, 348 bp, synthetic construct
  misc_feature 1..348: note = Synthetic: Heavy Chain - > 491-2A10 (LJI_0491-106).1, Nt
  source 1..348: mol_type = other DNA
caggtacagctcgtggagtctggaggaggcttgatccagcctggggggtccctgagactctcctgtgcagcctctgagttcatcgtcagtagcaactacatgagctgggtccgccaggctccagggaaggggctggagtgggtctcacttctttatagcggtggtaccacatactacgcagactccgtgaagggccgattcaccatctccagagacaattccaagaacacactgtttcttcaaatgaacagcctgagagccgaagacacggccatgtattactgtgcgagagataggggtccgtggttacatgactactggggccagggaaccctggtcaccgtctcc

SEQ ID NO: 101 - protein, 8 aa, synthetic construct
  REGION 1..8: note = Synthetic: Heavy Chain - > 491-2A10 (LJI_0491-106).2, CRDH1
EFIVSSNY

SEQ ID NO: 102 - protein, 7 aa, synthetic construct
  REGION 1..7: note = Synthetic: Heavy Chain - > 491-2A10 (LJI_0491-106).2, CRDH2
LYSGGTT

SEQ ID NO: 103 - protein, 11 aa, synthetic construct
  REGION 1..11: note = Synthetic: Heavy Chain - > 491-2A10 (LJI_0491-106).2, CRDH3
ARDRGPWLHDY

SEQ ID NO: 104 - protein, 116 aa, synthetic construct
  REGION 1..116: note = Synthetic: Heavy Chain - > 491-2A10 (LJI_0491-106).2, AA
EVQLVESGGGLIQPGGSLRLSCAASEFIVSSNYMRWVRQAPGKGLEWVSLLYSGGTTYYADSVKGRFTISRDNSKNTLFLQMNSLRAEDTAMYYCARDRGPWLHDYWGQGTLVTVS

SEQ ID NO: 105 - DNA, 348 bp, synthetic construct
  misc_feature 1..348: note = Synthetic: Heavy Chain - > 491-2A10 (LJI_0491-106).2, Nt
  source 1..348: mol_type = other DNA
gaggtgcagctggtggagtctggaggaggcttgatccagcctggggggtccctgagactttcctgtgcagcctctgagttcatcgtcagtagcaactacatgagatgggtccgccaggctccagggaaggggctggagtgggtctcacttctttatagcggtggtaccacatactacgcagactccgtgaagggccgattcaccatctccagagacaattccaagaacacactgtttcttcaaatgaacagcctgagagccgaagacacggccatgtattactgtgcgagagataggggtccgtggttacatgactactggggccagggaaccctggtcaccgtctcc

SEQ ID NO: 106 - protein, 6 aa, synthetic construct
  REGION 1..6: note = Synthetic: Light Chain - > 491-2A10 (LJI_0491-106).1, CDRL1
QDINKY

SEQ ID NO: 107 - skipped

SEQ ID NO: 108 - protein, 9 aa, synthetic construct
  REGION 1..9: note = Synthetic: Light Chain - > 491-2A10 (LJI_0491-106).1, CDRL3
QQYDNLPPT

SEQ ID NO: 109 - protein, 107 aa, synthetic construct
  REGION 1..107: note = Synthetic: Light Chain - > 491-2A10 (LJI_0491-106).1, AA
DIVMTQSPSSLSASVGDRVTITCQASQDINKYCNWYQQKPGKAPKLLIYDASNLETGVPSRFSGSGSGTDFTFTINSLQPEDIATYYCQQYDNLPPTFGGGTKVEIK

SEQ ID NO: 110 - DNA, 321 bp, synthetic construct
  misc_feature 1..321: note = Synthetic: Light Chain - > 491-2A10 (LJI_0491-106).1, Nt
  source 1..321: mol_type = other DNA
gacatcgtgatgacccagtctccatcctccctgtctgcatctgtaggagacagagtcaccatcacttgccaggcgagtcaggacattaacaagtattgcaattggtatcagcagaaaccagggaaagcccctaaactcctgatctacgatgcatccaatttggaaacaggggtcccatcaaggttcagtggaagtggatctgggacagactttactttcaccatcaacagcctgcagcctgaagatattgcaacatattactgtcaacaatatgataatctccctcccactttcggcggagggaccaaggtggaaatcaaa

SEQ ID NO: 111 - protein, 8 aa, synthetic construct
  REGION 1..8: note = Synthetic: Heavy Chain - > 491-2B1 (LJI_0491-109).1, CRDH1
GYTFTSYD

SEQ ID NO: 112 - protein, 8 aa, synthetic construct
  REGION 1..8: note = Synthetic: Heavy Chain - > 491-2B1 (LJI_0491-109).1, CRDH2
MNPNSGNT

SEQ ID NO: 113 - protein, 23 aa, synthetic construct
  REGION 1..23: note = Synthetic: Heavy Chain - > 491-2B1 (LJI_0491-109).1, CRDH3
ARGRYFDWLKGIEDYEEDRVGNY

SEQ ID NO: 114 - protein, 129 aa, synthetic construct
  REGION 1..129: note = Synthetic: Heavy Chain - > 491-2B1 (LJI_0491-109).1, AA
EVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARGRYFDWLKGIEDYEEDRVGNYWGQGTLVTVS

SEQ ID NO: 115 - DNA, 387 bp, synthetic construct
  misc_feature 1..387: note = Synthetic: Heavy Chain - > 491-2B1 (LJI_0491-109).1, Nt
  source 1..387: mol_type = other DNA
gaagtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagaggcagatattttgactggttaaagggaatagaggactacgaagaagatagggtggggaactactggggccagggaaccctggtcaccgtctcc

SEQ ID NO: 116 - protein, 6 aa, synthetic construct
  REGION 1..6: note = Synthetic: Light Chain - > 491-2B1 (LJI_0491-109).1, CDRL1
QSVSSY

SEQ ID NO: 117 - skipped

SEQ ID NO: 118 - protein, 10 aa, synthetic construct
  REGION 1..10: note = Synthetic: Light Chain - > 491-2B1 (LJI_0491-109).1, CDRL3
QQRSNWPPLT

SEQ ID NO: 119 - protein, 108 aa, synthetic construct
  REGION 1..108: note = Synthetic: Light Chain - > 491-2B1 (LJI_0491-109).1, AA
DIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 120 - DNA, 324 bp, synthetic construct
  misc_feature 1..324: note = Synthetic: Light Chain - > 491-2B1 (LJI_0491-109).1, Nt
  source 1..324: mol_type = other DNA
gacattgtgctgacccagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgctcactttcggcggagggaccaaggtggagatcaaa

SEQ ID NO: 121 - protein, 8 aa, synthetic construct
  REGION 1..8: note = Synthetic: Heavy Chain - > 491-2B1 (LJI_0491-109).2, CRDH1
GYTFTSYD

SEQ ID NO: 122 - protein, 8 aa, synthetic construct
  REGION 1..8: note = Synthetic: Heavy Chain - > 491-2B1 (LJI_0491-109).2, CRDH2
MNPNSGNT

SEQ ID NO: 123 - protein, 23 aa, synthetic construct
  REGION 1..23: note = Synthetic: Heavy Chain - > 491-2B1 (LJI_0491-109).2, CRDH3
ARGRYFDWLKGIEDYVEDRVGNY

SEQ ID NO: 124 - protein, 129 aa, synthetic construct
  REGION 1..129: note = Synthetic: Heavy Chain - > 491-2B1 (LJI_0491-109).2, AA
QVQLVQSGAEVKKPGASVKVSCKASGYTFTSYDINWVRQATGQGLEWMGWMNPNSGNTGYAQKFQGRVTMTRNTSISTAYMELSSLRFEDTAVYYCARGRYFDWLKGIEDYVEDRVGNYWGQGTLVTVS

SEQ ID NO: 125 - DNA, 387 bp, synthetic construct
  misc_feature 1..387: note = Synthetic: Heavy Chain - > 491-2B1 (LJI_0491-109).2, Nt
  source 1..387: mol_type = other DNA
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatttgaggacacggccgtgtattactgtgcgagaggcagatattttgactggttaaagggaatagaggactacgtagaagatagggtggggaactactggggccagggaaccctggtcaccgtctcc

SEQ ID NO: 126 - protein, 6 aa, synthetic construct
  REGION 1..6: note = Synthetic: Light Chain - > 491-2B1 (LJI_0491-109).2, CDRL1
QSVSSY

SEQ ID NO: 127 - skipped

SEQ ID NO: 128 - protein, 10 aa, synthetic construct
  REGION 1..10: note = Synthetic: Light Chain - > 491-2B1 (LJI_0491-109).2, CDRL3
QQRSNWPPLT

SEQ ID NO: 129 - protein, 108 aa, synthetic construct [sequence omitted]

SEQ ID NO: 130 - DNA, 324 bp, synthetic construct; identical to SEQ ID NO: 95
  misc_feature 1..324: note = Synthetic: Light Chain - > 491-2B1 (LJI_0491-109).2, Nt
  source 1..324: mol_type = other DNA

SEQ ID NO: 131 - protein, 8 aa, synthetic construct
  REGION 1..8: note = Synthetic: Heavy Chain - > 491-2B5 (LJI_0491-113), CRDH1
GGSFSGYY

SEQ ID NO: 132 - protein, 7 aa, synthetic construct
  REGION 1..7: note = Synthetic: Heavy Chain - > 491-2B5 (LJI_0491-113), CRDH2
INHSGST

SEQ ID NO: 133 - protein, 12 aa, synthetic construct
  REGION 1..12: note = Synthetic: Heavy Chain - > 491-2B5 (LJI_0491-113), CRDH3
ARVVIPFFYFDY

SEQ ID NO: 134 - protein, 117 aa, synthetic construct
  REGION 1..117: note = Synthetic: Heavy Chain - > 491-2B5 (LJI_0491-113), AA
QVQLQQWGAGLLKPSETLSLTCAVYGGSFSGYYWSWIRQPPGKGLEWIGEINHSGSTNYNPSLKSRVTISVDTSKNHFSLKLSSVTAADTAVYYCARVVIPFFYFDYWGQGTLVTVS

SEQ ID NO: 135 - DNA, 351 bp, synthetic construct
  misc_feature 1..351: note = Synthetic: Heavy Chain - > 491-2B5 (LJI_0491-113), Nt
  source 1..351: mol_type = other DNA
caggtgcagctacaacagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtggacacgtccaagaaccacttctccctgaagctgagctctgtgaccgccgcggacacggctgtgtattactgtgcgagagtggtaattccttttttctactttgactactggggccagggaaccctggtcaccgtctcc

SEQ ID NO: 136 - protein, 5 aa, synthetic construct
  REGION 1..5: note = Synthetic: Light Chain - > 491-2B5 (LJI_0491-113), CDRL1
NIGSK

SEQ ID NO: 137 - skipped

SEQ ID NO: 138 - protein, 11 aa, synthetic construct
  REGION 1..11: note = Synthetic: Light Chain - > 491-2B5 (LJI_0491-113), CDRL3
QVWDSSDPLYV

SEQ ID NO: 139 - protein, 108 aa, synthetic construct
  REGION 1..108: note = Synthetic: Light Chain - > 491-2B5 (LJI_0491-113), AA
QAGLTQPPSVSVAPGKTARITCGGNNIGSKSVHWYQQKPGQAPVLVIYYDSDRPSGIPERFSGSNSGNTATLTISRVEAGDEADYYCQVWDSSDPLYVFGTGTKLTVL

SEQ ID NO: 140 - DNA, 324 bp, synthetic construct
  misc_feature 1..324: note = Synthetic: Light Chain - > 491-2B5 (LJI_0491-113), Nt
  source 1..324: mol_type = other DNA
caggcagggctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtgatcctctttatgtcttcggaactgggaccaagcttaccgtccta

SEQ ID NO: 141 - protein, 8 aa, synthetic construct
  REGION 1..8: note = Synthetic: Heavy Chain - > 491-2B7 (LJI_0491-115).1, CRDH1
GFIFSSYA

SEQ ID NO: 142 - protein, 8 aa, synthetic construct
  REGION 1..8: note = Synthetic: Heavy Chain - > 491-2B7 (LJI_0491-115).1, CRDH2
ISGSGGST

SEQ ID NO: 143 - protein, 17 aa, synthetic construct
  REGION 1..17: note = Synthetic: Heavy Chain - > 491-2B7 (LJI_0491-115).1, CRDH3
AKQCRTDGVCYTALFDY

SEQ ID NO: 144 - protein, 123 aa, synthetic construct
  REGION 1..123: note = Synthetic: Heavy Chain - > 491-2B7 (LJI_0491-115).1, AA
EVQLVQSGGGLVQPGGSLRLSCAASGFIFSSYAMSWVRQAPGKGLEWVSAISGSGGSTYYADSVKGRFTISRDNSKNTLYLQMNSLRAEDTAVYYCAKQCRTDGVCYTALFDYWGQGSLVTVS

SEQ ID NO: 145 - DNA, 369 bp, synthetic construct
  misc_feature 1..369: note = Synthetic: Heavy Chain - > 491-2B7 (LJI_0491-115).1, Nt
  source 1..369: mol_type = other DNA
gaggtgcagctggtgcagtctgggggaggcttggtacagcctggggggtccctgagactctcctgtgcagcctctggattcatctttagcagctatgccatgagctgggtccgccaggctccagggaaggggctggagtgggtctcagctattagtggtagtggtggtagcacatactacgcagactccgtgaagggccgattcaccatctccagagacaattccaagaacacgctgtatctgcaaatgaacagcctgagagccgaggacacggccgtttattactgtgcgaaacaatgtagaactgatggtgtatgctatacggctttatttgactactggggccagggaagcctggtcaccgtctcc

SEQ ID NO: 146 - protein, 8 aa, synthetic construct
  REGION 1..8: note = Synthetic: Heavy Chain - > 491-2B7 (LJI_0491-115).2, CRDH1
GFIFSSYA

SEQ ID NO: 147 - protein, 8 aa, synthetic construct
  REGION 1..8: note = Synthetic: Heavy Chain - > 491-2B7 (LJI_0491-115).2, CRDH2
ISGSGGST

SEQ ID NO: 148 - protein, 17 aa, synthetic construct
  REGION 1..17: note = Synthetic: Heavy Chain - > 491-2B7 (LJI_0491-115).2, CRDH3
AKQCRTDGVCYTALFDY

SEQ ID NO: 149 - protein, 123 aa, synthetic construct
  REGION 1..123: note = Synthetic: Heavy Chain - > 491-2B7 (LJI_0491-115).2, AA
EVQLLESGGGLVQPGGSLRLSCAASGFIFSSYAMSWVRQAPGKGLEWVSAISGSGGSTYYADSVKGRFTISRDNSKNTLYLQMNSLRAEDTAVYYCAKQCRTDGVCYTALFDYWGQGSLVTVS

SEQ ID NO: 150 - DNA, 369 bp, synthetic construct
  misc_feature 1..369: note = Synthetic: Heavy Chain - > 491-2B7 (LJI_0491-115).2, Nt
  source 1..369: mol_type = other DNA
gaggtgcagctgttggagtctgggggaggcttggtacagcctggggggtccctgagactctcctgtgcagcctctggattcatctttagcagctatgccatgagctgggtccgccaggctccagggaaggggctggagtgggtctcagctattagtggtagtggtggtagcacatactacgcagactccgtgaagggccgattcaccatctccagagacaattccaagaacacgctgtatctgcaaatgaacagcctgagagccgaggacacggccgtttattactgtgcgaaacaatgtagaactgatggtgtatgctatacggctttatttgactactggggccagggaagcctggtcaccgtctcc

SEQ ID NO: 151 - protein, 8 aa, synthetic construct
  REGION 1..8: note = Synthetic: Light Chain - > 491-2B7 (LJI_0491-115), CDRL1
SSNIGSNY

SEQ ID NO: 152 - skipped

SEQ ID NO: 153 - protein, 12 aa, synthetic construct
  REGION 1..12: note = Synthetic: Light Chain - > 491-2B7 (LJI_0491-115), CDRL3
AAWDDSLSGRWV

SEQ ID NO: 154 - protein, 111 aa, synthetic construct
  REGION 1..111: note = Synthetic: Light Chain - > 491-2B7 (LJI_0491-115), AA
QPVLTQPPSASGTPGQRVTISCSGSSSNIGSNYVYWYQQLPGTAPKLLIDGNNQRPSGVPDRFSGSKSGTSASLAISGLRSEDEADYYCAAWDDSLSGRWVFGGGTKLTVL

SEQ ID NO: 155 - DNA, 333 bp, synthetic construct
  misc_feature 1..333: note = Synthetic: Light Chain - > 491-2B7 (LJI_0491-115), Nt
  source 1..333: mol_type = other DNA
cagcctgtgctgactcagccaccctcagcgtctgggacccccgggcagagggtcaccatctcttgttccggaagcagttccaacatcggaagtaattatgtatactggtaccagcagctcccaggaacggcccccaaactcctcatcgatgggaataatcagcggccctcaggggtccctgaccgattctctggctccaagtctggcacctcagcctccctggccatcagtgggctccggtccgaggatgaggctgattattactgtgcagcatgggatgacagcctgagtggccgttgggtgttcggcggagggaccaagcttaccgtccta

SEQ ID NO: 156 - protein, 8 aa, synthetic construct
  REGION 1..8: note = Synthetic: Heavy Chain - > 491-2B8 (LJI_0491-116).1, CRDH1
GYTFTTFG

SEQ ID NO: 157 - protein, 8 aa, synthetic construct
  REGION 1..8: note = Synthetic: Heavy Chain - > 491-2B8 (LJI_0491-116).1, CRDH2
ISTYNGNT

SEQ ID NO: 158 - protein, 21 aa, synthetic construct
  REGION 1..21: note = Synthetic: Heavy Chain - > 491-2B8 (LJI_0491-116).1, CRDH3
TRGGSRYNWNDFDYYNYYMDV

SEQ ID NO: 159 - protein, 127 aa, synthetic construct
  REGION 1..127: note = Synthetic: Heavy Chain - > 491-2B8 (LJI_0491-116).1, AA
QVQLVQSGAEVKKPGASVKVSCKASGYTFTTFGISWVRQAPGQGLEWMGWISTYNGNTKYAQKLQGRVTMTTDTSTSTAYMELRSLRSDDTAVYFCTRGGSRYNWNDFDYYNYYMDVWGKGTTVTVS

SEQ ID NO: 160 - DNA, 381 bp, synthetic construct
  misc_feature 1..381: note = Synthetic: Heavy Chain - > 491-2B8 (LJI_0491-116).1, Nt
  source 1..381: mol_type = other DNA
caggttcagctggtgcagtctggagctgaggtgaagaagcctggggcctcagtgaaggtctcctgcaaggcttcaggttacacctttaccacctttggtatcagctgggtgcgacaggcccctggacaagggcttgagtggatgggatggatcagcacttacaatggtaacacaaagtatgcacagaagctccagggcagagtcaccatgaccacagacacatccacgagcacagcctacatggagctgaggagcctgagatctgacgacacggccgtgtatttctgtacgagagggggctctaggtataactggaacgacttcgactactacaactactacatggacgtctggggcaaagggaccacggtcaccgtctcc

SEQ ID NO: 161 - protein, 11 aa, synthetic construct
  REGION 1..11: note = Synthetic: Light Chain - > 491-2B8 (LJI_0491-116).1, CDRL1
QSLVYSDGNTY

SEQ ID NO: 162 - skipped

SEQ ID NO: 163 - protein, 8 aa, synthetic construct
  REGION 1..8: note = Synthetic: Light Chain - > 491-2B8 (LJI_0491-116).1, CDRL3
MQGTHWPF

SEQ ID NO: 164 - protein, 111 aa, synthetic construct
  REGION 1..111: note = Synthetic: Light Chain - > 491-2B8 (LJI_0491-116).1, AA
EIVMTQSPLSLPVTLGQPASISCRSSQSLVYSDGNTYLNWFQQRPGQSPRRLIYKVSNRDSGVPDRFSGSGSGTNFTLKISRVEAEDVGVYYCMQGTHWPFFGGGTKVEIK

SEQ ID NO: 165 - DNA, 333 bp, synthetic construct
  misc_feature 1..333: note = Synthetic: Light Chain - > 491-2B8 (LJI_0491-116).1, Nt
  source 1..333: mol_type = other DNA
gaaattgtaatgacacagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactaatttcacactgaaaatcagcagggtggaggctgaggatgttggggtttattactgcatgcaaggtacacactggccttttttcggcggagggaccaaggtggaaatcaaa

SEQ ID NO: 166 - protein, 8 aa, synthetic construct
  REGION 1..8: note = Synthetic: Heavy Chain - > 491-2B8 (LJI_0491-116).2, CRDH1
GYTFTTFG

SEQ ID NO: 167 - protein, 8 aa, synthetic construct
  REGION 1..8: note = Synthetic: Heavy Chain - > 491-2B8 (LJI_0491-116).2, CRDH2
ISTYNGNT

SEQ ID NO: 168 - protein, 21 aa, synthetic construct
  REGION 1..21: note = Synthetic: Heavy Chain - > 491-2B8 (LJI_0491-116).2, CRDH3
TRGGSRYNWYDFDYYNYYMDV

SEQ ID NO: 169 - protein, 127 aa, synthetic construct
  REGION 1..127: note = Synthetic: Heavy Chain - > 491-2B8 (LJI_0491-116).2, AA
QVQLVQSGAEVKKPGASVKVSCKASGYTFTTFGISWVRQAPGQGLEWMGWISTYNGNTKYAQKLQGRVTMTTDTSTSTAYMELRSLRSDDTAVYFCTRGGSRYNWYDFDYYNYYMDVWGKGTTVTVS

SEQ ID NO: 170 - DNA, 381 bp, synthetic construct
  misc_feature 1..381: note = Synthetic: Heavy Chain - > 491-2B8 (LJI_0491-116).2, Nt
  source 1..381: mol_type = other DNA
caggtgcagctggtgcagtctggagctgaggtgaagaagcctggggcctcagtgaaggtctcctgcaaggcttcaggttacacctttaccacctttggtatcagctgggtgcgacaggcccctggacaagggcttgagtggatgggatggatcagcacttacaatggtaacacaaagtatgcacagaagctccagggcagagtcaccatgaccacagacacatccacgagcacagcctacatggagctgaggagcctgagatctgacgacacggccgtgtatttctgtacgagagggggctctaggtataactggtacgacttcgactactacaactactacatggacgtctggggcaaagggaccacggtcaccgtctcc

SEQ ID NO: 171 - protein, 11 aa, synthetic construct
  REGION 1..11: note = Synthetic: Light Chain - > 491-2B8 (LJI_0491-116).2, CDRL1
QSLVYSDGNTY

SEQ ID NO: 172 - skipped

SEQ ID NO: 173 - protein, 8 aa, synthetic construct
  REGION 1..8: note = Synthetic: Light Chain - > 491-2B8 (LJI_0491-116).2, CDRL3
MQGTHWPF

SEQ ID NO: 174 - protein, 111 aa, synthetic construct
  REGION 1..111: note = Synthetic: Light Chain - > 491-2B8 (LJI_0491-116).2, AA
DVVMTQSPLSLPVTLGQPASISCRSSQSLVYSDGNTYLNWFQQRPGQSPRRLIYKVSNRDSGVPDRFSGSGSGTNFTLKISRVEAEDVGVYYCMQGTHWPFFGGGTKVEIK

SEQ ID NO: 175 - DNA, 333 bp, synthetic construct
  misc_feature 1..333: note = Synthetic: Light Chain - > 491-2B8 (LJI_0491-116).2, Nt
  source 1..333: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactaatttcacactgaaaatcagcagggtggaggctgaggatgttggggtttattactgcatgcaaggtacacactggccttttttcggcggagggaccaaggtggagatcaaa

SEQ ID NO: 176 - protein, 7 aa, synthetic construct
  REGION 1..7: note = Synthetic: Heavy Chain - > 491-2G3 (LJI_0491-171), CRDH1
FTFDEYA

SEQ ID NO: 177 - protein, 8 aa, synthetic construct
  REGION 1..8: note = Synthetic: Heavy Chain - > 491-2G3 (LJI_0491-171), CRDH2
ISWNSGRL

SEQ ID NO: 178 - protein, 18 aa, synthetic construct
  REGION 1..18: note = Synthetic: Heavy Chain - > 491-2G3 (LJI_0491-171), CRDH3
AKSSRFLEWLLPGDAFDI

SEQ ID NO: 179 - protein, 124 aa, synthetic construct
  REGION 1..124: note = Synthetic: Heavy Chain - > 491-2G3 (LJI_0491-171), AA
QVQLVESGGGWVQPGRSRRLSCAASGFTFDEYAMHWVRQTPGKGLEWVSGISWNSGRLDYADSVKGRFTISRDNAKNSLYLQMNSLRAEDTALYYCAKSSRFLEWLLPGDAFDIWGRGTMVSVS

SEQ ID NO: 180 - DNA, 372 bp, synthetic construct
  misc_feature 1..372: note = Synthetic: Heavy Chain - > 491-2G3 (LJI_0491-171), Nt
  source 1..372: mol_type = other DNA
caggtacagctcgtggagtctgggggaggctgggtacagcctggcaggtcccggagactctcctgtgcagcctctggattcacctttgatgagtatgccatgcactgggtccgacaaactccagggaagggcctggagtgggtctcaggtattagttggaatagtggtcgcctagactatgcggactctgtgaagggccgattcaccatctccagagacaacgccaagaactccctgtatctgcaaatgaacagtctgagagctgaggacacggccttgtattactgtgcaaaatcttcacgatttttggagtggttattgcccggtgatgcttttgatatctggggccgagggacaatggtctccgtctct

SEQ ID NO: 181 - protein, 6 aa, synthetic construct
  REGION 1..6: note = Synthetic: Light Chain - > 491-2G3 (LJI_0491-171).1, CDRL1
QSVSDY

SEQ ID NO: 182 - skipped

SEQ ID NO: 183 - protein, 10 aa, synthetic construct
  REGION 1..10: note = Synthetic: Light Chain - > 491-2G3 (LJI_0491-171).1, CDRL3
QQRSNWPPIT

SEQ ID NO: 184 - protein, 108 aa, synthetic construct
  REGION 1..108: note = Synthetic: Light Chain - > 491-2G3 (LJI_0491-171).1, AA
DIVLTQSPATLSLSPGERATLSCRSSQSVSDYLAWYQQKPGQAPRLLIYDASNRATGIPARFSGSGSGTDFTLTISSLEPEDFAVYYCQQRSNWPPITFGQGTRLEIK

SEQ ID NO: 185 - DNA, 324 bp, synthetic construct
  misc_feature 1..324: note = Synthetic: Light Chain - > 491-2G3 (LJI_0491-171).1, Nt
  source 1..324: mol_type = other DNA
gacattgtgctgacccagtctccagccaccctgtctttgtctccaggggaaagagccaccctctcctgcaggtccagtcagagtgttagcgactacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcaacagcgtagcaactggcctccgatcaccttcggccaagggacacgactggagattaaa

SEQ ID NO: 186 - protein, 6 aa, synthetic construct
  REGION 1..6: note = Synthetic: Light Chain - > 491-2G3 (LJI_0491-171).2, CDRL1
QSVSDY

SEQ ID NO: 187 - skipped

SEQ ID NO: 188 - protein, 10 aa, synthetic construct
  REGION 1..10: note = Synthetic: Light Chain - > 491-2G3 (LJI_0491-171).2, CDRL3
QQRSNWPPIT

SEQ ID NO: 189 - protein, 108 aa, synthetic construct
  REGION 1..108: note = Synthetic: Light Chain - > 491-2G3 (LJI_0491-171).2, AA
EIVLTQSPATLSLSPGERATLSCRSSQSVSDYLAWYQQKPGQAPRLLIYDASNRATGIPARFSGSGSGTDFTLTISSLEPEDFAVYYCQQRSNWPPITFGQGTRLEIK

SEQ ID NO: 190 - DNA, 324 bp, synthetic construct
  misc_feature 1..324: note = Synthetic: Light Chain - > 491-2G3 (LJI_0491-171).2, Nt
  source 1..324: mol_type = other DNA
gaaattgtgttgacacagtctccagccaccctgtctttgtctccaggggaaagagccaccctctcctgcaggtccagtcagagtgttagcgactacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcaacagcgtagtaactggcctccgatcaccttcggccaagggacacgactggagattaaa

SEQ ID NO: 191 - protein, 8 aa, synthetic construct
  REGION 1..8: note = Synthetic: Heavy Chain - > 491-4H4 (LJI_0491-278), CRDH1
GFTVSSNY

SEQ ID NO: 192 - protein, 6 aa, synthetic construct
  REGION 1..6: note = Synthetic: Heavy Chain - > 491-4H4 (LJI_0491-278), CRDH2
YSGGST

SEQ ID NO: 193 - protein, 9 aa, synthetic construct
  REGION 1..9: note = Synthetic: Heavy Chain - > 491-4H4 (LJI_0491-278), CRDH3
ARAPYGDSH

SEQ ID NO: 194 - protein, 115 aa, synthetic construct
  REGION 1..115: note = Synthetic: Heavy Chain - > 491-4H4 (LJI_0491-278), AA
QVQLVESGGGLIQPGGSLRLSCVASGFTVSSNYMSWFRQGPGKGLEWVSVIYSGGSTFHADSVKGRFTISRDHSKNTLYLQMNSLRAEDTAVYYCARAPYGDSHWGQGTLVTVSS

SEQ ID NO: 195 - DNA, 346 bp, synthetic construct
  misc_feature 1..346: note = Synthetic: Heavy Chain - > 491-4H4 (LJI_0491-278), Nt
  source 1..346: mol_type = other DNA
caggtacagctcgtggagtctggaggaggcttgatccagcctggggggtccctgagactctcctgtgtagcctctgggttcaccgtcagtagcaactacatgagctggttccgccagggtccagggaaggggctggagtgggtctcagttatttatagcggtggtagcacattccatgcagactccgtgaagggccgattcaccatctccagagaccattccaagaacacgctgtatcttcaaatgaacagcctgagagccgaggacacggccgtgtattactgtgcgagagctccctacggtgactcccactggggccagggaaccctggtcaccgtctcctcag

SEQ ID NO: 196 - protein, 6 aa, synthetic construct
  REGION 1..6: note = Synthetic: Light Chain - > 491-4H4 (LJI_0491-278), CDRL1
QDIRNF

SEQ ID NO: 197 - skipped

SEQ ID NO: 198 - protein, 9 aa, synthetic construct
  REGION 1..9: note = Synthetic: Light Chain - > 491-4H4 (LJI_0491-278), CDRL3
QQYDNLPIT

SEQ ID NO: 199 - protein, 107 aa, synthetic construct
  REGION 1..107: note = Synthetic: Light Chain - > 491-4H4 (LJI_0491-278), AA
DIVMTQSPSSLSASIGDRVTITCQASQDIRNFLNWYQQKPGKAPKLLIHDASNLETGVPSRFSGSGSGTDFTFTISSLQPEDIATYYCQQYDNLPITFGQGTRLEIK

SEQ ID NO: 200 - DNA, 321 bp, synthetic construct
  misc_feature 1..321: note = Synthetic: Light Chain - > 491-4H4 (LJI_0491-278), Nt
  source 1..321: mol_type = other DNA
gatattgtgatgactcagtctccatcctccctgtctgcatctataggagacagagtcaccatcacttgccaggcgagtcaggacattcggaactttttaaattggtatcagcagaaaccagggaaagcccctaagctcctgatccacgatgcatccaatttggaaacaggggtcccatcaaggttcagtggaagtggatctgggacagattttactttcaccatcagcagcctgcagcctgaagatattgcaacatattactgtcaacagtatgataatctccccatcaccttcggccaagggacacgactggagattaaa

SEQ ID NO: 201 - protein, 8 aa, synthetic construct
  REGION 1..8: note = Synthetic: HC CDR 27
GFTFDDYA

SEQ ID NO: 202 - protein, 8 aa, synthetic construct
  REGION 1..8: note = Synthetic: HC CDR 27
SSWNSGSV

SEQ ID NO: 203 - protein, 19 aa, synthetic construct
  REGION 1..19: note = Synthetic: HC CDR 27
AKAVDPTRGSYSPDYGFDI

SEQ ID NO: 204 - protein, 8 aa, synthetic construct
  REGION 1..8: note = Synthetic: HC CDR 39
GFTFSGSA

SEQ ID NO: 205 - protein, 10 aa, synthetic construct
  REGION 1..10: note = Synthetic: HC CDR 39
IRSKANSYAT

SEQ ID NO: 206 - protein, 13 aa, synthetic construct
  REGION 1..13: note = Synthetic: HC CDR 39
TREDIVGVPVAKD

SEQ ID NO: 207 - protein, 6 aa, synthetic construct
  REGION 1..6: note = Synthetic: LC CDR 29 / 39
QSISSY

SEQ ID NO: 208 - skipped

SEQ ID NO: 209 - protein, 9 aa, synthetic construct
  REGION 1..9: note = Synthetic: LC CDR 29
QQSYSTPLT

SEQ ID NO: 210 - protein, 10 aa, synthetic construct
  REGION 1..10: note = Synthetic: LC CDR 39
QQSYSTLSIT

SEQ ID NO: 211 - protein, 125 aa, synthetic construct
  REGION 1..125: note = Synthetic: LJI_491_27_HC (1C3)
QVQLLESGGGLVQPGRSLRLSCAASGFTFDDYAMHWVRQPPGKGLEWVSGSSWNSGSVVYADSVKGRFTISRDSAKNSLHLQMNSLRVEDTALYYCAKAVDPTRGSYSPDYGFDIWGQGTMVTVS

SEQ ID NO: 212 - DNA, 375 bp, synthetic construct
  misc_feature 1..375: note = Synthetic: LJI_491_27_HC (1C3)
  source 1..375: mol_type = other DNA
gaagtgcagctggtggagtttgggggaggcttggtacagcctggcaggtccctgagactttcctgtgcagcctttggattcacctttgatgattatgccatgcactgggtccggcaacctccagggaagggcctggagtgggtttcaggtagtagttggaatagtggtagcgtagtctatgcggactctgtgaagggccgattcaccatctccagagacagcgccaagaactccctgcatttgcaaatgaacagtttgagagttgaggacacggccttatattactgtgcaaaagccgtcgaccctacacgtgggagctactccccagattatggttttgatatctggggccaagggacaatggtcaccgtctct

SEQ ID NO: 213 - protein, 110 aa, synthetic construct
  REGION 1..110: note = Synthetic: LJI_491_27_kLC (1C3)
DIQMTQSPSSLSASVGDRVTITCRASQSISSYLNWYQQKPGKAPNLLIYAASSLESGVPSRFSGSGSGTDFTLTISSLQPEDFATYYCQQSYSTPLTFGGGTKVEIKRTV

SEQ ID NO: 214 - DNA, 312 bp, synthetic construct
  misc_feature 1..312: note = Synthetic: LJI_491_27_kLC (1C3)
  source 1..312: mol_type = other DNA
gacatccagatgacccagtctccatcctccctgtctgcatctgtaggagacagagtcaccatcacttgccgggcaagtcagagcatcagcagctatttaaattggtatcagcagaaaccagggaaagcccctaacctcctgatctatgctgcatccagtttggaaagtggggtcccatcaaggttcagtggcagtggatctgggacagatttcactctcaccatcagcagcctgcaacctgaagattttgcaacttactactgtcaacagagttacagtaccccgctcactttcggcggagggaccaaggtg

SEQ ID NO: 215 - protein, 124 aa, synthetic construct
  REGION 1..124: note = Synthetic: LJI_491_39_HC (2E6)
EVQLVQSGGGLVQPGGSLKLSCAASGFTFSGSAMHWVRQASGKGLEWVGHIRSKANSYATAYAASKGRFTISRDDSKNTAYLQMNSLKTEDTAVYYCTREDIVGVPVAKDWFDPWGQGTLVTVS

SEQ ID NO: 216 - DNA, 372 bp, synthetic construct
  misc_feature 1..372: note = Synthetic: LJI_491_39_HC (2E6)
  source 1..372: mol_type = other DNA
gaagtgcagctggtgcagtctgggggaggcttggtccagcctggggggtccctgaaactctcctgtgcagcctctgggttcaccttcagtggctctgctatgcactgggtccgccaggcttccgggaaagggctggagtgggttggccatattagaagcaaagctaacagttacgcgacagcatatgctgcgtcggtgaaaggcaggttcaccatctccagagatgattcaaagaacacggcgtatctgcaaatgaacagcctgaataccgaggacacggccgtgtattactgtactagagaggatattgtaggagtaccagttgctaaggactggttcgacccctggggccagggaaccctggtcaccgtc

SEQ ID NO: 217 - protein, 111 aa, synthetic construct
  REGION 1..111: note = Synthetic: LJI_491_39_kLC (2E6)
DIQMTQSPSSLFASVGDRVTITCRASQSISSYLNWYQQKPGKAPKLLIYAASSLQSGVPSRFSGSGFGTDFTLTISSLQPEDFATYYCQQSYSTLSITFGQGTRLEIKRTV

SEQ ID NO: 218 - DNA, 333 bp, synthetic construct
  misc_feature 1..333: note = Synthetic: LJI_491_39_kLC (2E6)
  source 1..333: mol_type = other DNA
gacatccagatgacgcagtctccatcctccctgtttgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatttgggacagatttcactctcaccatcagcagtctgcaacctgaagattttgcaacttactactgtcaacagagttacagtaccctttcgatcaccttcggccaagggacacgactggagattaaacgaactgtg

SEQ ID NO: 219 - protein, 8 aa, synthetic construct
  REGION 1..8: note = Synthetic: Heavy Chain Complementarity Determining Regions , CDRH1
GFTFSYYW

SEQ ID NO: 220 - protein, 9 aa, synthetic construct
  REGION 1..9: note = Synthetic: Heavy Chain Complementarity Determining Regions , CDRH2
IKQDGSEKY

SEQ ID NO: 221 - protein, 16 aa, synthetic construct
  REGION 1..16: note = Synthetic: Heavy Chain Complementarity Determining Regions , CDRH3
ARDRYGWPSIPAPFDY

SEQ ID NO: 222 - protein, 9 aa, synthetic construct
  REGION 1..9: note = Synthetic: Light Chain Complementarity Determining Regions, CDRL1
SSDIGGYDY

SEQ ID NO: 223 - protein, 4 aa, synthetic construct
  REGION 1..4: note = Synthetic: Light Chain Complementarity Determining Regions, CDRL2
DVSL

SEQ ID NO: 224 - protein, 11 aa, synthetic construct
  REGION 1..11: note = Synthetic: Light Chain Complementarity Determining Regions, CDRL3
NSYTNISTLGV

SEQ ID NO: 225 - protein, 122 aa, synthetic construct
  REGION 1..122: note = Synthetic: 996-C5_HC
QVQLVESGGGLVQPGGSLRLSCAASGFTFSYYWMSWVRQAPGKGLEWVANIKQDGSEKYYVDSVKGRFTISRDNAKNSLYLQMNSLRAEDTAVYYCARDRYGWPSIPAPFDYWGQGTLVTVS

SEQ ID NO: 226 - DNA, 369 bp, synthetic construct
  misc_feature 1..369: note = Synthetic: Heavy Chain
  source 1..369: mol_type = other DNA
caagttcaattggtggagagcgggggagggttggttcaaccaggcggcagtctgcgcttgtcctgtgcggcatcagggtttaccttcagctactactggatgagttgggtgcggcaggctccaggaaaaggacttgaatgggtagcaaatattaaacaggatgggagcgaaaaatactacgtcgactccgtcaaaggaagattcactataagccgggataacgcgaaaaactctttgtaccttcaaatgaactctctcagagctgaggataccgccgtctattattgtgccagggatcggtatgggtggccaagtatcccagcgccctttgattactggggccaagggactcttgttaccgtgtctagc

SEQ ID NO: 227 - protein, 111 aa, synthetic construct
  REGION 1..111: note = Synthetic: 996-C5_LLC of the present invention
QSALTQPASVSGSPGQSITISCTGTSSDIGGYDYVSWYQHHPGKAPKLMIYDVSLRPSGVSNRFSGSKSGNTASLTISGLQAEDEADYYCNSYTNISTLGVFGGGTKLTVL

SEQ ID NO: 228 - protein, 111 aa, synthetic construct
  REGION 1..111: note = Synthetic: 996-C5 (Parent antibody)
QSALTQPASVSGSPGQSITISCTGTSSDIGGYDYVSWYQHHPGKAPKLMIYDVSNRPSGVSNRFSGSKSGNTASLTISGLQAEDEADYYCNSYTNISTLGVFGGGTKLTVL

SEQ ID NO: 229 - DNA, 330 bp, synthetic construct
  misc_feature 1..330: note = Synthetic: 996-C5_LLC
  source 1..330: mol_type = other DNA
caatcagcgcttacccagcctgcaagtgttagcgggagcccagggcagtctataacaatttcctgtactggtacaagctccgacatcggggggtacgattatgtcagttggtaccaacaccatcccggcaaagctccaaaacttatgatttacgacgtaagccttcgacccagcggggtgagcaacaggttcagtgggtcaaagagcgggaacacagcctcacttacaataagtggacttcaggctgaggatgaggctgactattactgtaatagttatactaacatttcaacgcttggggtattcgggggcggtactaagctgacagtc

SEQ ID NO: 230 - protein, 50 aa, synthetic construct
  REGION 1..50: note = Synthetic: linker
  VARIANT 6..50: note = "GGGGS" may or may not be present
GGGGSGGGGSGGGGSGGGGSGGGGSGGGGSGGGGSGGGGSGGGGSGGGGS

SEQ ID NO: 231 - protein, 4 aa, synthetic construct
  REGION 1..4: note = Synthetic: linker
GPGP

SEQ ID NO: 232 - protein, 6 aa, synthetic construct
  REGION 1..6: note = Synthetic: linker
GGSGGS

SEQ ID NO: 233 - protein, 21 aa, synthetic construct
  REGION 1..21: note = Synthetic: CDRH3
CAKAVDPTRGSYSPDYGFDIW

SEQ ID NO: 234 - protein, 13 aa, synthetic construct
  REGION 1..13: note = Synthetic: CDRH3
CGRDRGPWLHDYW

SEQ ID NO: 235 - protein, 11 aa, synthetic construct
  REGION 1..11: note = Synthetic: CDRH3
CARAPYGDSHW

SEQ ID NO: 236 - protein, 21 aa, synthetic construct
  REGION 1..21: note = Synthetic: CDRH3
CAKDSGRKQLWGREDYYMGVW

SEQ ID NO: 237 - protein, 19 aa, synthetic construct
  REGION 1..19: note = Synthetic: CDRH3
CTREDIVGVPVAKDWFDPW

SEQ ID NO: 238 - protein, 11 aa, synthetic construct [sequence omitted]

SEQ ID NO: 239 - protein, 11 aa, synthetic construct [sequence omitted]

SEQ ID NO: 240 - protein, 11 aa, synthetic construct [sequence omitted]

SEQ ID NO: 241 - protein, 13 aa, synthetic construct [sequence omitted]

SEQ ID NO: 242 - protein, 12 aa, synthetic construct [sequence omitted]

SEQ ID NO: 243 - protein, 30 aa, synthetic construct
  REGION 1..30: note = Synthetic: linker
  VARIANT 4..30: note = "GGS" may or may not be present
GGSGGSGGSGGSGGSGGSGGSGGSGGSGGS

(19) United States
(12) Patent Application Publication
(10) Pub. No.: US 2025/0382353 A1
(43) Pub. Date: Dec. 18, 2025

(54) ANTI-SARS-COV2 SPIKE (S) ANTIBODIES AND USES THEREOF

(71) Applicant: La Jolla Institute for Immunology, La Jolla, CA (US)

(72) Inventors: Kathryn M. Hastie, La Jolla, CA (US); Erica Ollmann Saphire, La Jolla, CA (US); Stephanie Harkins, La Jolla, CA (US)

(21) Appl. No.: 18/838,692

(22) PCT Filed: Feb. 17, 2023
(86) PCT No.: PCT/US2023/062831

§ 371 Date: Aug. 15, 2024

Related U.S. Application Data
(60) Provisional application No. 63/311,656, filed on Feb. 18, 2022.
(60) Provisional application No. 63/313,598, filed on Feb. 24, 2022.
(60) Provisional application No. 63/316,669, filed on Mar. 4, 2022.
(60) Provisional application No. 63/319,955, filed on Mar. 15, 2022.

(51) Int. Cl. C07K 16/10; A61K 39/00; G01N 33/569

(52) U.S. Cl. CPC C07K 16/1003; G01N 33/56983; A61K 2039/505; C07K 2317/53; C07K 2317/55; C07K 2317/76; C07K 2317/92; G01N 2333/165; G01N 2469/10

(57) ABSTRACT

The present invention includes monoclonal antibody or antigen-binding fragment thereof, methods of using, detection, recombinant vectors, host cells, kits, variants, and pharmaceutical compositions that include the antibody or antigen-binding fragment thereof that is cross-reactive and binds to different variants of a Spike protein of SARS-CoV-2 (SARS2-S).

### CROSS-REFERENCE TO RELATED APPLICATIONS

This application is the National Stage of International Application No. PCT/US2023/062831, filed on Feb. 17, 2023, which claims priority to U.S. Provisional Application Ser. No. 63/311,656, filed Feb. 18, 2022; 63/313,598 filed Feb. 24, 2022; 63/316,669, filed Mar. 4, 2022; and 63/319,955, filed Mar. 15, 2022, the entire contents of each of which are incorporated herein by reference.

### TECHNICAL FIELD OF THE INVENTION

The present invention relates in general to the field of antibodies against coronavirus, and more particularly, to human antibodies that bind the Spike protein of SARS-CoV-2 (SARS2-S) and its variants.

### STATEMENT OF FEDERALLY-FUNDED RESEARCH

None.

### INCORPORATION-BY-REFERENCE OF MATERIALS FILED ON COMPACT DISC

The Sequence Listing in an XML file, named as LJII2019WO.xml of 313,762 bytes, created on Apr. 5, 2023 and submitted to the United States Patent and Trademark Office via Patent Center, is incorporated herein by reference.

### BACKGROUND OF THE INVENTION

Without limiting the scope of the invention, its background is described in connection with SARS-CoV-2.

The worldwide spread of SARS-CoV-2 in the human population resulted in the ongoing COVID-19 pandemic that has already caused more than 600 million infections and more than 6.8 million deaths. To initiate infection, the SARS-CoV-2 spike (S) glycoprotein promotes binding to ACE2 located on the surface of the host cell, initiating a cascade of conformational changes in the protein that drives from a metastable pre-fusion conformation to a stable post-fusion conformation. That reorganization of the protein exposes the fusion peptide and a fusion between the viral and host membranes is driven by the S2 chain of the proprotein. Given its external location on the virus and its functionality, SARS-CoV-2 spike ‘S’ protein in its pre-fusion state is one target of neutralizing antibodies and therefore the main target of the design of safe and effective therapies.

Moreover, since the beginning of the pandemic, the SARS-CoV-2 spike ‘S’ protein has shown a significant number of new variants, with a new variant emerging every few months. What is needed are improved antibodies against SARS-CoV-2, including those that are cross-reactive to more than one variant of the SARS-CoV-2 spike ‘S’ protein. Also needed are antibodies that bind to the S-protein in both the “up” oriented receptor-binding domains (RBD) of S-protein and the “down” oriented RBDs of S-protein.

### SUMMARY OF THE INVENTION

As embodied and broadly described herein, an aspect of the present disclosure relates to a monoclonal antibody or antigen-binding fragment thereof comprising heavy chain complementarity determining regions (CDR) H1, CDRH2, and CDRH3, and light chain CDRL1, CDRL2, and CDRL3 of: SEQ ID NOS: 1, 2, 3 and 6, 7, 8; SEQ ID NOS: 11, 12, 13 and 16, 17, 18; SEQ ID NOS: 21, 22, 23 and 36, 37, 38; or 26, 27, 28 and 36, 37, 38; or 31, 32, 33 and 36, 37, 38; SEQ ID NOS: 41, 42, 43 and 51, 52, 53; or 46, 47, 48 and 51, 52, 53; SEQ ID NOS: 56, 57, 58 and 61, 62, 63; SEQ ID NOS: 66, 67, 68 and 71, 72, 73; SEQ ID NOS: 76, 77, 78 and 81, 82, 83; SEQ ID NOS: 86, 87, 88 and 91, 92, 93; SEQ ID NOS: 96, 97, 98 and 106, 107, 108; or 101, 102, 103 and 106, 107, 108; SEQ ID NOS: 111, 112, 113 and 116, 117, 118; SEQ ID NOS: 121, 122, 123 and 126, 127, 128; SEQ ID NOS: 131, 132, 133 and 136, 137, 138; SEQ ID NOS: 141, 142, 143 and 151, 152, 153; or 146, 147, 148 and 151, 152, 153; SEQ ID NOS: 156, 157, 158 and 161, 162, 163; SEQ ID NOS: 166, 167, 168 and 171, 172, 173; SEQ ID NOS: 176, 177, 178 and 181, 182, 183; or 176, 177, 178 and 186, 187, 188; SEQ ID NOS: 191, 192, 193 and 196, 197, and 198; SEQ ID NOS: 201, 202, 203 or 204, 205, 206 and 207, 206, 208 or 207, 206, 210; or SEQ ID NOS: 219, 220, 221, and 222, 223, and 224 respectively. In one aspect, the antibody or antigen-binding fragment is cross-reactive to one or more, and in some cases binds to one or more, or at least two, variants of a Spike protein of SARS-CoV-2 (SARS2-S), and wherein the antibody is a neutralizing antibody. In another aspect, the antibody heavy chain variable region and light chain variable region comprises an amino acid sequence at least about 90, 91, 92, 93, 94, 95, 96, 97, 98, 99, or 100% identical to the amino acid sequence of SEQ ID NOS: 4 and 9; 14 and 19; 24 and 39, or 29 and 39, or 34 and 39; 44 and 54, or 49 and 54; 59 and 64; 69 and 74; 79 and 84; 89 and 94; 99 and 109, or 104 and 109; 114 and 119; 124 and 129; 134 and 139; 144 and 154, or 149 and 154; 159 and 164; 169 and 174; 179 and 184, or 179 and 189; 194 and 199, 211 and 213, 215 and 217, 225 and 226, or 225 and 227, respectively. In another aspect, the antigen-binding fragment is a recombinant single-chain fragment variable (scFV) antibody, Fab fragment, F(ab′)₂ fragment, or Fv fragment. In another aspect, the antibody or antigen-binding fragment is chimeric, humanized, fully human, or bispecific. In another aspect, the antibody or antigen-binding fragment comprises an Fc portion mutated to at least one of: eliminate or enhance Fc Receptor (FcR) interactions to change a half-life, increase or decrease antibody-dependent cellular cytotoxicity, or increase or decrease complement activation. In another aspect, the antibody heavy chain variable region and light chain variable region is encoded by a nucleic acid sequence having at least 90, 91, 92, 93, 94, 95, 96, 97, 98, 99, or 100% identical to a nucleic acid of SEQ ID NOS: 5 and 10; 15 and 20; 25 and 40, or 30 or 40, or 35 or 40; 45 and 55, or 46 and 55; 60 and 65; 70 and 75; 80 and 85; 90 and 95; 100 and 110 or 105 and 110; 115 and 120; 125 and 130; 135 and 140; 145 and 155, or 150 and 155; 160 and 165; 170 and 175; 180 and 185, or 180 and 190; 195 and 200, 212 and 214, 216 and 218, 226 and 229, respectively. In another aspect, the antibody or antigen-binding fragment is adapted for administration or genetic delivery with an RNA or DNA sequence or vector encoding the antibody or antigen-binding fragment. In another aspect, the monoclonal antibody further comprises a peptide linker in a hinge region of the heavy chain.

As embodied and broadly described herein, an aspect of the present disclosure relates to a pharmaceutical composition comprising a pharmaceutically acceptable excipient and a therapeutically effective amount of a monoclonal antibody or antigen-binding fragment thereof comprising heavy chain complementarity determining regions (CDR) H1, CDRH2, and CDRH3, and light chain CDRL1, CDRL2, and CDRL3 of: SEQ ID NOS: 1, 2, 3 and 6, 7, 8; SEQ ID NOS: 11, 12, 13 and 16, 17, 18; SEQ ID NOS: 21, 22, 23 and 36, 37, 38; or 26, 27, 28 and 36, 37, 38; or 31, 32, 33 and 36, 37, 38; SEQ ID NOS: 41, 42, 43 and 51, 52, 53; or 46, 47, 48 and 51, 52, 53; SEQ ID NOS: 56, 57, 58 and 61, 62, 63; SEQ ID NOS: 66, 67, 68 and 71, 72, 73; SEQ ID NOS: 76, 77, 78 and 81, 82, 83; SEQ ID NOS: 86, 87, 88 and 91, 92, 93; SEQ ID NOS: 96, 97, 98 and 106, 107, 108; or 101, 102, 103 and 106, 107, 108; SEQ ID NOS: 111, 112, 113 and 116, 117, 118; SEQ ID NOS: 121, 122, 123 and 126, 127, 128; SEQ ID NOS: 131, 132, 133 and 136, 137, 138; SEQ ID NOS: 141, 142, 143 and 151, 152, 153; or 146, 147, 148 and 151, 152, 153; SEQ ID NOS: 156, 157, 158 and 161, 162, 163; SEQ ID NOS: 166, 167, 168 and 171, 172, 173; SEQ ID NOS: 176, 177, 178 and 181, 182, 183; or 176, 177, 178 and 186, 187, 188; SEQ ID NOS: 191, 192, 193 and 196, 197, and 198; SEQ ID NOS: 201, 202, 203 or 204, 205, 206 and 207, 206, 208 or 207, 206, 210; or SEQ ID NOS: 219, 220, 221, and 222, 223, and 224, respectively. In one aspect, the antibody or antigen-binding fragment is cross-reactive to one or more, and in some cases at least two variants, of a Spike protein of SARS-CoV-2 (SARS2-S), and wherein the antibody is a neutralizing antibody. In another aspect, the antibody heavy chain variable region and light chain variable region comprises an amino acid sequence at least about 90, 91, 92, 93, 94, 95, 96, 97, 98, 99, or 100% identical to the amino acid sequence of SEQ ID NOS: 4 and 9; 14 and 19; 24 and 39, or 29 and 39, or 34 and 39; 44 and 54, or 49 and 54; 59 and 64; 69 and 74; 79 and 84; 89 and 94; 99 and 109, or 104 and 109; 114 and 119; 124 and 129; 134 and 139; 144 and 154, or 149 and 154; 159 and 164; 169 and 174; 179 and 184, or 179 and 189, 194 and 199, 211 and 213, 215 and 217, 225 and 226, or 225 and 227, respectively. In another aspect, the antigen-binding fragment is a recombinant single-chain fragment variable (scFV) antibody, Fab fragment, F(ab′)₂ fragment, or Fv fragment. In another aspect, the antibody or antigen-binding fragment is chimeric, humanized, fully human, or bispecific. In another aspect, the e antibody or antigen-binding fragment comprises an Fc portion mutated to at least one of: eliminate or enhance Fc Receptor (FcR) interactions to change a half-life, increase or decrease antibody-dependent cellular cytotoxicity, or increase or decrease complement activation. In another aspect, the antibody heavy chain variable region and light chain variable region is encoded by a nucleic acid sequence having at least 90, 91, 92, 93, 94, 95, 96, 97, 98, 99, or 100% identical to a nucleic acid of SEQ ID NOS: 5 and 10; 15 and 20; 25 and 40, or 30 or 40, or 35 or 40; 45 and 55, or 46 and 55; 60 and 65; 70 and 75; 80 and 85; 90 and 95; 100 and 110 or 105 and 110; 115 and 120; 125 and 130; 135 and 140; 145 and 155, or 150 and 155; 160 and 165; 170 and 175; 180 and 185, or 180 and 190; 195 and 200, 212 and 214, 216 and 218, 226 and 229, respectively. In another aspect, the antibody further comprises a peptide linker in a hinge region between the variable and constant domains of the heavy chain. In another aspect, the antibody or antigen-binding fragment is adapted for administration or genetic delivery with an RNA or DNA sequence or vector encoding the antibody or antigen-binding fragment. In another aspect, the antibody or antigen-binding fragment is formulated for nasal, pulmonary, alveolar, intravenous, administration.

As embodied and broadly described herein, an aspect of the present disclosure relates to a method of treating SARS-CoV-2 in a subject in need thereof, the method comprising treating SARS-CoV-2 in a subject in need thereof, the method comprising administering to the subject a monoclonal antibody or antigen-binding fragment thereof comprising heavy chain complementarity determining regions (CDR) H1, CDRH2, and CDRH3, and light chain CDRL1, CDRL2, and CDRL3 of: SEQ ID NOS: 1, 2, 3 and 6, 7, 8; SEQ ID NOS: 11, 12, 13 and 16, 17, 18; SEQ ID NOS: 21, 22, 23 and 36, 37, 38; or 26, 27, 28 and 36, 37, 38; or 31, 32, 33 and 36, 37, 38; SEQ ID NOS: 41, 42, 43 and 51, 52, 53; or 46, 47, 48 and 51, 52, 53; SEQ ID NOS: 56, 57, 58 and 61, 62, 63; SEQ ID NOS: 66, 67, 68 and 71, 72, 73; SEQ ID NOS: 76, 77, 78 and 81, 82, 83; SEQ ID NOS: 86, 87, 88 and 91, 92, 93; SEQ ID NOS: 96, 97, 98 and 106, 107, 108; or 101, 102, 103 and 106, 107, 108; SEQ ID NOS: 111, 112, 113 and 116, 117, 118; SEQ ID NOS: 121, 122, 123 and 126, 127, 128; SEQ ID NOS: 131, 132, 133 and 136, 137, 138; SEQ ID NOS: 141, 142, 143 and 151, 152, 153; or 146, 147, 148 and 151, 152, 153; SEQ ID NOS: 156, 157, 158 and 161, 162, 163; SEQ ID NOS: 166, 167, 168 and 171, 172, 173; SEQ ID NOS: 176, 177, 178 and 181, 182, 183; or 176, 177, 178 and 186, 187, 188; SEQ ID NOS: 191, 192, 193 and 196, 197, and 198; SEQ ID NOS: 201, 202, 203 or 204, 205, 206 and 207, 206, 208 or 207, 206, 210; or SEQ ID NOS: 219, 220, 221, and 222, 223, and 224 respectively. In one aspect, the antibody or antigen-binding fragment is cross-reactive to one or more, and in some cases at least two variants, of a Spike protein of SARS-CoV-2 (SARS2-S), and wherein the antibody is a neutralizing antibody. In another aspect, the antibody heavy chain variable region and light chain variable region comprises an amino acid sequence at least about 90, 91, 92, 93, 94, 95, 96, 97, 98, 99, or 100% identical to the amino acid sequence of SEQ ID NOS: 4 and 9; 14 and 19; 24 and 39, or 29 and 39, or 34 and 39; 44 and 54, or 49 and 54; 59 and 64; 69 and 74; 79 and 84; 89 and 94; 99 and 109, or 104 and 109; 114 and 119; 124 and 129; 134 and 139; 144 and 154, or 149 and 154; 159 and 164; 169 and 174; 179 and 184, or 179 and 189; 194 and 199, 211 and 213, 215 and 217, 225 and 226, or 225 and 227, respectively. In another aspect, the antigen-binding fragment is a recombinant single-chain fragment variable (scFV) antibody, Fab fragment, F(ab′)₂ fragment, or Fv fragment. In another aspect, the antibody or antigen-binding fragment is chimeric, humanized, fully human, or bispecific. In another aspect, the antibody or antigen-binding fragment comprises an Fc portion mutated to at least one of: eliminate or enhance Fc Receptor (FcR) interactions to change a half-life, increase or decrease antibody-dependent cellular cytotoxicity, or increase or decrease complement activation. In another aspect, the antibody heavy chain variable region and light chain variable region is encoded by a nucleic acid sequence having at least 90, 91, 92, 93, 94, 95, 96, 97, 98, 99, or 100% identical to a nucleic acid of SEQ ID NOS: 5 and 10; 15 and 20; 25 and 40, or 30 or 40, or 35 or 40; 45 and 55, or 46 and 55; 60 and 65; 70 and 75; 80 and 85; 90 and 95; 100 and 110 or 105 and 110; 115 and 120; 125 and 130; 135 and 140; 145 and 155, or 150 and 155; 160 and 165; 170 and 175; 180 and 185, or 180 and 190; 195 and 200; 212 and 214; 216 and 218; or 226 and 229, respectively. In another aspect, the antibody or antigen-binding fragment is adapted for administration or genetic delivery with an RNA or DNA sequence or vector encoding the antibody or antigen-binding fragment.

As embodied and broadly described herein, an aspect of the present disclosure relates to a pharmaceutical device suitable for nasal or pulmonary delivery of monoclonal antibody or antigen-binding fragment thereof comprising heavy chain complementarity determining regions (CDR) H1, CDRH2, and CDRH3, and light chain CDRL1, CDRL2, and CDRL3 of: SEQ ID NOS: 1, 2, 3 and 6, 7, 8; SEQ ID NOS: 11, 12, 13 and 16, 17, 18; SEQ ID NOS: 21, 22, 23 and 36, 37, 38; or 26, 27, 28 and 36, 37, 38; or 31, 32, 33 and 36, 37, 38; SEQ ID NOS: 41, 42, 43 and 51, 52, 53; or 46, 47, 48 and 51, 52, 53; SEQ ID NOS: 56, 57, 58 and 61, 62, 63; SEQ ID NOS: 66, 67, 68 and 71, 72, 73; SEQ ID NOS: 76, 77, 78 and 81, 82, 83; SEQ ID NOS: 86, 87, 88 and 91, 92, 93; SEQ ID NOS: 96, 97, 98 and 106, 107, 108; or 101, 102, 103 and 106, 107, 108; SEQ ID NOS: 111, 112, 113 and 116, 117, 118; SEQ ID NOS: 121, 122, 123 and 126, 127, 128; SEQ ID NOS: 131, 132, 133 and 136, 137, 138; SEQ ID NOS: 141, 142, 143 and 151, 152, 153; or 146, 147, 148 and 151, 152, 153; SEQ ID NOS: 156, 157, 158 and 161, 162, 163; SEQ ID NOS: 166, 167, 168 and 171, 172, 173; SEQ ID NOS: 176, 177, 178 and 181, 182, 183; or 176, 177, 178 and 186, 187, 188; SEQ ID NOS: 191, 192, 193 and 196, 197, and 198; SEQ ID NOS: 201, 202, 203 or 204, 205, 206 and 207, 206, 208 or 207, 206, 210; or SEQ ID NOS: 219, 220, 221, and 222, 223, and 224, respectively, selected from an inhaler for liquids, a nebulizer, metered-dose inhaler, aerosol, and a dry powder inhaler. In one aspect, the antibody or antigen-binding fragment is cross-reactive to one or more, and in some cases at least two, variants of a Spike protein of SARS-CoV-2 (SARS2-S).

As embodied and broadly described herein, an aspect of the present disclosure relates to a method for detecting a variant of a Spike protein of SARS-CoV-2 (SARS2-S) comprising: obtaining or having obtained a biological sample suspected of comprising a SARS-CoV-2 virus; contacting the biological sample with a monoclonal antibody or antigen-binding fragment thereof comprising heavy chain complementarity determining regions (CDR) H1, CDRH2, and CDRH3, and light chain CDRL1, CDRL2, and CDRL3 of: SEQ ID NOS: 1, 2, 3 and 6, 7, 8; SEQ ID NOS: 11, 12, 13 and 16, 17, 18; SEQ ID NOS: 21, 22, 23 and 36, 37, 38; or 26, 27, 28 and 36, 37, 38; or 31, 32, 33 and 36, 37, 38; SEQ ID NOS: 41, 42, 43 and 51, 52, 53; or 46, 47, 48 and 51, 52, 53; SEQ ID NOS: 56, 57, 58 and 61, 62, 63; SEQ ID NOS: 66, 67, 68 and 71, 72, 73; SEQ ID NOS: 76, 77, 78 and 81, 82, 83; SEQ ID NOS: 86, 87, 88 and 91, 92, 93; SEQ ID NOS: 96, 97, 98 and 106, 107, 108; or 101, 102, 103 and 106, 107, 108; SEQ ID NOS: 111, 112, 113 and 116, 117, 118; SEQ ID NOS: 121, 122, 123 and 126, 127, 128; SEQ ID NOS: 131, 132, 133 and 136, 137, 138; SEQ ID NOS: 141, 142, 143 and 151, 152, 153; or 146, 147, 148 and 151, 152, 153; SEQ ID NOS: 156, 157, 158 and 161, 162, 163; SEQ ID NOS: 166, 167, 168 and 171, 172, 173; SEQ ID NOS: 176, 177, 178 and 181, 182, 183; or 176, 177, 178 and 186, 187, 188; SEQ ID NOS: 191, 192, 193 and 196, 197, and 198; SEQ ID NOS: 201, 202, 203 or 204, 205, 206 and 207, 206, 208 or 207, 206, 210; or SEQ ID NOS: 219, 220, 221, and 222, 223, and 224, respectively; and detecting the binding of the antibody or antigen-binding fragment to the SARS-CoV-2 virus. In one aspect, the step of detecting the antibody or antigen-binding fragment is diagnostic for the detection of SARS-CoV-2 in the biological sample. In another aspect, the method further comprises detecting SARS-CoV-2 by performing an immunoassay on the biological sample from a subject; wherein the immunoassay uses an antibody or antigen-binding fragment. In another aspect, the immunoassay selected from radioimmunoassay, enzyme-linked immunosorbent assay (ELISA), sandwich assays, Western blot, immunoprecipitation, immunohistochemistry, immunofluorescence, antibody microarray, dot blotting, and fluorescence-activated cell sorting (FACS).

As embodied and broadly described herein, an aspect of the present disclosure relates to a kit for detecting one or more variants of a Spike protein of SARS-CoV-2 comprising a monoclonal antibody or antigen-binding fragment thereof comprising heavy chain complementarity determining regions (CDR) H1, CDRH2, and CDRH3, and light chain CDRL1, CDRL2, and CDRL3 of: SEQ ID NOS: 1, 2, 3 and 6, 7, 8; SEQ ID NOS: 11, 12, 13 and 16, 17, 18; SEQ ID NOS: 21, 22, 23 and 36, 37, 38; or 26, 27, 28 and 36, 37, 38; or 31, 32, 33 and 36, 37, 38; SEQ ID NOS: 41, 42, 43 and 51, 52, 53; or 46, 47, 48 and 51, 52, 53; SEQ ID NOS: 56, 57, 58 and 61, 62, 63; SEQ ID NOS: 66, 67, 68 and 71, 72, 73; SEQ ID NOS: 76, 77, 78 and 81, 82, 83; SEQ ID NOS: 86, 87, 88 and 91, 92, 93; SEQ ID NOS: 96, 97, 98 and 106, 107, 108; or 101, 102, 103 and 106, 107, 108; SEQ ID NOS: 111, 112, 113 and 116, 117, 118; SEQ ID NOS: 121, 122, 123 and 126, 127, 128; SEQ ID NOS: 131, 132, 133 and 136, 137, 138; SEQ ID NOS: 141, 142, 143 and 151, 152, 153; or 146, 147, 148 and 151, 152, 153; SEQ ID NOS: 156, 157, 158 and 161, 162, 163; SEQ ID NOS: 166, 167, 168 and 171, 172, 173; SEQ ID NOS: 176, 177, 178 and 181, 182, 183; or 176, 177, 178 and 186, 187, 188; SEQ ID NOS: 191, 192, 193 and 196, 197, and 198; SEQ ID NOS: 201, 202, 203 or 204, 205, 206 and 207, 206, 208 or 207, 206, 210; or SEQ ID NOS: 219, 220, 221, and 222, 223, and 224, respectively, for the isolation and/or detection of SARS-CoV-2 in one or more sample types of human or nonhuman origin, including: blood, plasma, serum, saliva, tears, cerebrospinal fluid, lymph, urine, feces, exhaled breath condensate, perspiration, amniotic fluid, exosomes, cell and tissue lysates.

As embodied and broadly described herein, an aspect of the present disclosure relates to a kit for concurrent detection of two or more variants of a Spike protein of SARS-CoV-2 comprising a monoclonal antibody or antigen-binding fragment thereof comprising heavy chain complementarity determining regions (CDR) H1, CDRH2, and CDRH3, and light chain CDRL1, CDRL2, and CDRL3 of: SEQ ID NOS: 1, 2, 3 and 6, 7, 8; SEQ ID NOS: 11, 12, 13 and 16, 17, 18; SEQ ID NOS: 21, 22, 23 and 36, 37, 38; or 26, 27, 28 and 36, 37, 38; or 31, 32, 33 and 36, 37, 38; SEQ ID NOS: 41, 42, 43 and 51, 52, 53; or 46, 47, 48 and 51, 52, 53; SEQ ID NOS: 56, 57, 58 and 61, 62, 63; SEQ ID NOS: 66, 67, 68 and 71, 72, 73; SEQ ID NOS: 76, 77, 78 and 81, 82, 83; SEQ ID NOS: 86, 87, 88 and 91, 92, 93; SEQ ID NOS: 96, 97, 98 and 106, 107, 108; or 101, 102, 103 and 106, 107, 108; SEQ ID NOS: 111, 112, 113 and 116, 117, 118; SEQ ID NOS: 121, 122, 123 and 126, 127, 128; SEQ ID NOS: 131, 132, 133 and 136, 137, 138; SEQ ID NOS: 141, 142, 143 and 151, 152, 153; or 146, 147, 148 and 151, 152, 153; SEQ ID NOS: 156, 157, 158 and 161, 162, 163; SEQ ID NOS: 166, 167, 168 and 171, 172, 173; SEQ ID NOS: 176, 177, 178 and 181, 182, 183; or 176, 177, 178 and 186, 187, 188; SEQ ID NOS: 191, 192, 193 and 196, 197, and 198; SEQ ID NOS: 201, 202, 203 or 204, 205, 206 and 207, 206, 208 or 207, 206, 210; or SEQ ID NOS: 219, 220, 221, and 222, 223, and 224, respectively, for the isolation and/or detection of SARS-CoV-2 in one or more sample types of human or nonhuman origin, including: blood, plasma, serum, saliva, tears, cerebrospinal fluid, lymph, urine, feces, exhaled breath condensate, perspiration, amniotic fluid, exosomes, cell and tissue lysates.

As embodied and broadly described herein, an aspect of the present disclosure relates to a recombinant nucleic acid molecule encoding an antibody or antigen-binding fragment thereof as described hereinabove. In one aspect, the nucleic acid molecule encoding the antigen-binding fragment thereof, wherein the antibody heavy chain variable region and light chain variable region is encoded by a nucleic acid sequence having at least 90, 91, 92, 93, 94, 95, 96, 97, 98, 99, or 100% identical to the nucleic acid of SEQ ID NOS: 5 and 10; 15 and 20; 25 and 40, or 30 or 40, or 35 or 40; 45 and 55, or 46 and 55; 60 and 65; 70 and 75; 80 and 85; 90 and 95; 100 and 110 or 105 and 110; 115 and 120; 125 and 130; 135 and 140; 145 and 155, or 150 and 155; 160 and 165; 170 and 175; 180 and 185, or 180 and 190; 195 and 200; 212 and 214; 216 and 218; or 226 and 229, respectively.

As embodied and broadly described herein, an aspect of the present disclosure relates to a recombinant expression vector comprising an expression control sequence operatively linked to the recombinant nucleic acid molecule.

As embodied and broadly described herein, an aspect of the present disclosure relates to a host cell comprising the recombinant nucleic acid molecule.

As embodied and broadly described herein, an aspect of the present disclosure relates to a method of making an antibody or antigen-binding fragment of an antibody comprising culturing a recombinant host cell comprising a recombinant expression construct comprising an expression control sequence operatively linked to a recombinant nucleic acid molecule encoding a monoclonal antibody or binding fragment thereof comprising heavy chain complementarity determining regions (CDR) H1, CDRH2, and CDRH3, and light chain CDRL1, CDRL2, and CDRL3 of: SEQ ID NOS: 1, 2, 3 and 6, 7, 8; SEQ ID NOS: 11, 12, 13 and 16, 17, 18; SEQ ID NOS: 21, 22, 23 and 36, 37, 38; or 26, 27, 28 and 36, 37, 38; or 31, 32, 33 and 36, 37, 38; SEQ ID NOS: 41, 42, 43 and 51, 52, 53; or 46, 47, 48 and 51, 52, 53; SEQ ID NOS: 56, 57, 58 and 61, 62, 63; SEQ ID NOS: 66, 67, 68 and 71, 72, 73; SEQ ID NOS: 76, 77, 78 and 81, 82, 83; SEQ ID NOS: 86, 87, 88 and 91, 92, 93; SEQ ID NOS: 96, 97, 98 and 106, 107, 108; or 101, 102, 103 and 106, 107, 108; SEQ ID NOS: 111, 112, 113 and 116, 117, 118; SEQ ID NOS: 121, 122, 123 and 126, 127, 128; SEQ ID NOS: 131, 132, 133 and 136, 137, 138; SEQ ID NOS: 141, 142, 143 and 151, 152, 153; or 146, 147, 148 and 151, 152, 153; SEQ ID NOS: 156, 157, 158 and 161, 162, 163; SEQ ID NOS: 166, 167, 168 and 171, 172, 173; SEQ ID NOS: 176, 177, 178 and 181, 182, 183; or 176, 177, 178 and 186, 187, 188; SEQ ID NOS: 191, 192, 193 and 196, 197, and 198; SEQ ID NOS: 201, 202, 203 or 204, 205, 206 and 207, 206, 208 or 207, 206, 210; or SEQ ID NOS: 219, 220, 221, and 222, 223, and 224, respectively, wherein the host cell produces the antibody or antigen-binding fragment of an antibody, and isolating the antibody or antigen-binding fragment of an antibody from the host cell.

### BRIEF DESCRIPTION OF THE DRAWINGS

For a more complete understanding of the features and advantages of the present invention, reference is now made to the detailed description of the invention along with the accompanying figures and in which:

FIG. 1 shows the binding of antibodies disclosed herein to SARS-CoV-2 spike protein variants, domain (receptor-binding, RBD or N-terminal domain, NTD) or SARS-1 spike protein coated on ELISA microtiter plates.

FIGS. 2A and 2B show the results from infection was normalized to the average number of cells infected with the indicated pseudovirus incubated with PBS rather than monoclonal antibody. (FIG. 2A) Data are presented as the relative neutralization for each concentration of antibody. (FIG. 2B) Neutralization IC50s (displayed as ng/mL) were calculated using non-linear regression in GraphPad Prism 9.0.

FIG. 3 is a graph that shows the relative neutralization for each concentration of antibody in Vero monolayers infected with rVSV-SARS-CoV-2.

FIGS. 4A and 4B shows that the purified IgGs of each antibody was complexed with the ectodomain of SARS-CoV2 Spike. (FIG. 2A) Three antigen-binding fragments (Fab) of mAb 491-27 (1C3) (top left) bind to “up” oriented receptor-binding domains (RBD) of Spike. (FIG. 2B) Two Fabs of mAb 491-39 (2E6) (top right) are bound to “down” oriented RBDs of Spike.

FIG. 5 is a graph that shows 100% ACE2 blocking with the 0491_27 antibody of the present invention.

FIG. 6 is a graph that shows the high-affinity on-rate of the 0491_27 antibody of the present invention.

FIG. 7 shows a 3-dimensional representation of the receptor binding domain (RBD) of SARS-CoV-2 in complex with the antigen-binding fragment (Fab) of antibody 996-C5, determined to 2 Å resolution. The mutation of light chain residue N55 to leucine enhances the hydrophobic packing interaction with RBD residue L441 (gray).

FIG. 8 is a graph that shows the comparative neutralization of G614-pseudovirus particles comparing the 996-C5 antibody and the 996-C5 LC_N55L antibody of the present invention.

FIGS. 9A to 9C. Variant-related mutations on the SARS-CoV-2 Spike. (FIG. 9A) Spike primary structure with subunit domain boundaries denoted. SP: signal peptide; NTD: N-terminal domain; RBD: receptor-binding domain; SD1: subdomain 1; SD2: subdomain 2; S1/S2: furin cleavage site; S2′: S2 sub-cleavage site; FP: fusion peptide; CH: central helix; HR1: heptad repeat 1; CD: connector domain; HR2: heptad repeat 2; TM: transmembrane domain; CT: cytoplasmic tail. (FIG. 9B) Positions and substitutions of variant-related mutations located in the RBD, compared to the B.1 lineage. The World Health Organization variant designation and Pango lineage are indicated for each variant of concern (VOC). (FIG. 9C) Cartoon representation of the RBD and positions of variant-related mutations. Mutations shared among Omicron variants, but not with other VOCs are shaded accordingly. Those specific to BA.1 and BA.2 are shaded respectively. Mutations found in multiple VOCs are shaded throughout, as well, including N501Y. Positions that are less frequently mutated in multiple VOCs (e.g., L452 and R346) are shaded as well.

FIGS. 10A to 10D. Broadly neutralizing antibodies from a vaccinee. (FIG. 10A) The heavy (top) and light (bottom) germline, percent somatic hypermutation (SHM) and CDR3 sequence is shown for each bnAb. Middle column SEQ ID NOS: 233, 234, 235, 236, 237, and 238, right column SEQ ID NOS: 239, 240, 241, 242, 243, and 244, respectively. (FIG. 10B) Neutralization of SARS-CoV-2 pseudoviruses bearing the spike of the indicated VOC. Color coding corresponds to the key shown in (C). (FIG. 10C) IC₅₀ values for each neutralization curve in (B), calculated by nonlinear regression analysis. The limit of detection (L.O.D.) is shown as a dotted line and indicates the maximum antibody concentration used in the experiment. Error bars for panels B and C indicate the standard deviation from the mean for at least three independent experiments, each performed in technical duplicates. (FIG. 10D) Fold-change in IC50 for each antibody:variant pair relative to D614G.

FIGS. 11A to 11C. Neutralization and kinetic analysis of select variants of concern and Fab fragments. (FIG. 11A) Neutralization of SARS-CoV-2 pseudoviruses bearing the spike of the indicated VOC by the Fab of each indicated antibody. (FIG. 11B) IC₅₀ values for each neutralization curve, calculated by nonlinear regression analysis. The limit of detection (L.O.D.), shown as a dotted line, indicates the maximum concentration of antibody used in the experiment (5000 ng/mL). For panels A and B, error bars indicate the standard deviation from the mean of four experiments, each performed in technical duplicates. (FIG. 11C) SPR analysis of each Fab:Spike pair, as indicated. The Fab ligand used for each experiment remains constant across each row and the spike analyte remains constant for each column. The average K_{D} obtained from eight replicate experiments is shown above each plot. Raw data are colored gray, with the 1:1 fit colored according to the spike concentration. For 1H2, the dissociation was truncated to 450 secs to capture an accurate off rate.

FIGS. 12A to 12D. Cryo-EM analysis of the 1C3-spike complex and interactions between 1C3 and Omicron BA.1 RBD. (FIG. 12A) Model of cryogenic electron microscopy structure of 1C3 antigen-binding fragment (Fab) in complex with SARS-CoV-2 Omicron BA.1 spike (PDB: 8F0G). One copy of 1C3 Fab (shown as cartoon representation with the HC and LC shown in dark and light, respectively) simultaneously contacts two spike monomers (shown as a surface representation), A (gray), and B (light gray). (B) SARS-CoV-2 Omicron spike interactions with 1C3, a closeup view of 1C3 HC interacting with the spike RBD in monomer A. Dashed lines indicate hydrogen bonds. (FIG. 12C) Closeup view of 1C3 light chain interacting with spike RBD in monomers A and B. Dashed lines indicate hydrogen bonds. (FIG. 12D) RBD residue F486 in BA.1, which is mutated to Valine in BA.4/5, forms intensive hydrophobic contacts with 1C3 HC residues.

FIGS. 13A to 13G. Cryo-EM analysis of 2A10-1H2-spike complex and interactions between 2A10, 1H2 and D614G RBD. (FIG. 13A) Model of cryogenic electron microscopy structure of 1H2 and 2A10 antigen-binding fragments (Fab) in complex with SARS-CoV-2 D614G spike. The local refined structure of the RBD complexed with the two Fabs is modeled on the D614G spike model (shown as surface representation, PDB: 8F0H). Only one copy of each of the two Fabs is shown as a cartoon representation. 2A10 is shaded darker (HC) and lighter (LC), and 1H2 is also provided in dark (HC), and light (LC). (FIG. 13B-FIG. 13D) Interactions between 2A10 and D614G spike. 2A10 mainly utilizes heavy chain complementarity-determining region (CDR) H1 (B) and CDRH2 (C) and LC residues from CDR L2 and L3 (D) to contact the spike. Hydrogen bonds are indicated by dashed lines. Outlines highlight 2A10 residues that are mutated from germline as well as spike residues mutated in Omicron variants (K417N, T478K, N501Y, and Y505H). (FIG. 13E-FIG. 13G) Interactions between 1H2 and D614G spike. Contacts to the spike RBD by 1H2 residues from framework region (FR) 1, CDRL2, and FR3 are shown in (E). Zoomed-in views of contacts are shown in (F) for CDRL3 and in (G) for CDRH3. N343-linked glycans are shown as sticks. Hydrogen bonds are indicated by the dashed lines. 1H2 residue R84 is mutated from the germline and is outlined. Spike residues mutated in Omicron variants (R346K in BA.1.1, E484A) are also labeled with outlines.

FIGS. 14A to 14E. Antibody-mediated protection of BA.1 challenge in K18-huACE2 mice. (14A) Experiment design for B to D. Mice received a single 10 mg/kg injection of 1C3, 2A10, 1H2 or control anti-SARS1 mAb intraperitoneally (i.p.) on day −1 and were challenged intranasally (i.n.) with 10⁴ PFU of SARS CoV-2 BA.1 one day later (day 0). Tissues were harvested 3 days post-infection (dpi). (FIG. 14B) Infectious virus titers measured by plaque assay of lungs collected from mice challenged with 10⁴ PFU SARS CoV-2 BA.1 after passive transfer with mAb. Subgenomic 7a SARS CoV-2 RNA detected by RT-qPCR in nasal turbinate 3 dpi. (FIG. 14C) Lung images are representative of the distribution of immunofluorescence SARS CoV-2 N protein staining (magenta). (FIG. 14D) Representative histopathology images from lung samples stained with H&E. An arrow indicates bronchiolar epithelial cell necrosis. (FIG. 14E) Histopathological features of SARS CoV-2 infection in the lungs of mice were scored from 0 (least severe) to 5 (most severe). For panels B and E, data represent two independent experiments for a total of n=8-13 mice per group. Individual mice are represented as a square (male mouse) or circle (female mouse). Error bars indicate the SEM. Statistical analysis by Kruskal-Wallis test. *P<0.05, **P<0.01, and ***P<0.001. Only significant differences are shown. The limit of detection of each assay is represented by a dotted line.

FIGS. 15A to 15E. Antibody-mediated protection of BA.2 challenge in K18-huACE2 mice. (FIG. 15A) Experiment design for panels B to D. 4 groups of mice were injected via intraperitoneal injection (i.p.) with 10 mg/kg 1C3, 2A10, or 1H2, or 200 ul PBS on day −1. Mice were dosed intranasally (i.n.) with 10⁴ PFU of SARS CoV-2 BA.2 at day 0 and organs were harvested 3 days post-infection (dpi). (FIG. 15B) Infectious viral particles in lungs were titrated using plaque assay. Replicating virus detected by RT-qPCR of subgenomic 7a SARS CoV-2 RNA in nasal turbinate 3 dpi. (FIG. 15C) Immunofluorescent distribution of SARS CoV-2 N protein staining (magenta) of lungs. (FIG. 15D) Representative histopathology images from lung samples stained with H&E. Arrows indicate bronchiolar epithelial cell necrosis. (FIG. 15E) Histopathological features of SARS CoV-2 infection in the lungs of mice were scored from 0 (least severe) to 5 (most severe). Data for panels B and E represent two independent experiments for a total of n=9 mice per group, with each mouse represented as a square (male mouse) or circle (female mouse). Error bars indicate the SEM. Statistical analysis by Kruskal-Wallis test. *P<0.05, **P<0.01, and ***P<0.001. Only significant differences are indicated. Dotted line represents the limit of detection of each assay.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “antibody” refers to an intact antibody or a binding fragment thereof that binds specifically to a target antigen. Binding fragments are produced by recombinant DNA techniques, or by enzymatic or chemical cleavage of intact antibodies. Binding fragments include Fab, Fab′, F(ab′)₂, Fv, and single-chain variable fragment (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antigen-binding fragments (e.g., Fab, Fab′, F(ab′)₂, Fv, scFv) so long as they exhibit the desired biological activity. Antibodies (Abs) and immunoglobulins (Igs) are glycoproteins having the same structural characteristics. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial or eukaryotic cells, including plant cells.

As used herein, the terms “antibody fragment” or “antigen-binding fragment” refer to a portion of a full-length antibody, generally the antigen-binding or variable region, and include Fab, Fab′, F(ab′)₂, Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)₂ fragment that has two antigen-binding fragments which are capable of cross-linking antigen, and a residual other fragment (which is termed pFc′). As used herein, “functional fragment” with respect to antibodies, refers to Fv, F(ab) and F(ab′)₂ fragments.

As used herein, the “Fv” fragment is the minimum antigen-binding fragment that contains a complete antigen recognition and binding site. This region consists of a dimer of one heavy and one light chain variable domain in a tight, non-covalent association (V_{H}-V_{L} dimer).

It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antigen-binding fragment are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (V_{H}) followed by the constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.) The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted, while the antibodies' affinity for the SARS-COV2 spike proteins from different variants is retained. This engineering may only involve a few amino acids or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)₂, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fv framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

The presently disclosed and claimed invention further includes the use of fully human monoclonal antibodies cross-reactive against the SARS-COV2 spike proteins from different SARS-COV2 variants. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

An antibody or antigen-binding fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in WO/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved Clq binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine, and glycine. However, other residues can function as well. A random linker library was constructed in which the genes for the heavy and light chain variable domains were linked by a segment encoding an 18-amino acid polypeptide of variable composition. The scFv repertoire (approx. 5×10⁶ different members) is displayed on filamentous phage and subjected to affinity selection with hapten. The population of selected variants exhibited significant increases in binding activity but retained considerable sequence diversity. In certain embodiments, the antigen-binding fragments are further modified to increase their serum half-life by using modified Fc regions or mutations to the various constant regions, as are known in the art.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “substantially purified” refers to isolation of the antibodies or antigen-binding portions thereof of the present invention against SARS-CoV-2 such that the antibodies or antigen-binding portions comprise the majority percent of the sample in which it resides. Typically, in a sample, a substantially purified component comprises 50%, preferably 80%-85%, more preferably 90, 91, 92, 93, 94, 95, 96, 97, 98, or 99% of the sample. Techniques for purifying polynucleotides and polypeptides of interest are well-known in the art and include, for example, ion-exchange chromatography, affinity chromatography and sedimentation according to density.

As used herein, the term a “coding sequence” or a sequence which “encodes” the antibodies or antigen-binding portions thereof of the present invention against SARS-CoV-2, refers to a nucleic acid molecule that is transcribed (in the case of DNA) and translated (in the case of mRNA) into a polypeptide when placed under the control of appropriate regulatory sequences (or “control elements”) and in vitro or in vivo. The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

As used herein, the term “control elements”, includes, but is not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences, and/or sequence elements controlling an open chromatin structure.

As used herein, the term “nucleic acid” includes, but is not limited to, DNA or RNA that encodes the antibodies or antigen-binding portions thereof of the present invention against SARS-CoV-2 of the present invention, whether expressed or optimized for prokaryotic or eukaryotic expression. The term also captures sequences that include any of the known base analogs of DNA and RNA.

As used herein, the term “operably linked” refers to an arrangement of elements wherein the components so described are configured so as to perform their usual function. Thus, a given promoter operably linked to a coding sequence is capable of effecting the expression of the coding sequence when active. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

As used herein, the term “recombinant” refers to a polynucleotide that encodes the mutant SARS-CoV-2 spike antibody whether from the viral genome, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby and comparing this to a second amino acid sequence. In general, “identity” refers to an exact nucleotide to nucleotide or amino acid to amino acid correspondence of two polynucleotides or polypeptide sequences, respectively.

Two or more polynucleotide sequences can be compared by determining their “percent identity.” Two or more amino acid sequences likewise can be compared by determining their “percent identity.” The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986), relevant portion incorporated herein by reference. Suitable programs for calculating the percent identity or similarity between sequences are generally known in the art.

As used herein, the term a “vector” refers to a nucleic acid capable of transferring gene sequences to target cells (e.g., bacterial plasmid vectors, viral vectors, non-viral vectors, particulate carriers, and liposomes). Typically, “vector construct,” “expression vector,” and “gene transfer vector,” mean any nucleic acid construct capable of directing the expression of one or more sequences of interest in a host cell. Thus, the term includes cloning and expression vehicles, as well as viral vectors. The term is used interchangeable with the terms “nucleic acid expression vector” and “expression cassette.”

Many suitable expression systems are commercially available, including, for example, the following: baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portions of any of the above are incorporated herein by reference.

As used herein, the term “subject” refers to any member of the subphylum chordata, including, but not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

As used herein, the term “treatment” refers to any of (i) the prevention of infection or reinfection with a coronavirus using neutralizing antibodies, (ii) the reduction or elimination of symptoms, and (iii) the substantial or complete elimination of infection of a coronavirus or variants thereof. Treatment may be prophylactic (prior to infection) or therapeutic (following infection).

In certain embodiments, the coronavirus is SARS-CoV-2. In another aspect, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529 or its subvariants, BA.1, BA.2, and BA.3). In another aspect, the mutant coronavirus spike proteins are formed into dimers, trimers, multimers, or nanoparticles. In another aspect, the nanoparticles comprise ferritin nanoparticles, polymeric nanoparticles, or both.

As used herein, the term “effective dose” refers to that amount of the antibodies or antigen-binding portions thereof of the present invention against SARS-CoV-2 sufficient to induce immunity, to prevent and/or ameliorate an infection or to reduce at least one symptom of an infection and/or to enhance the efficacy of another dose of a SARS-CoV-2. An effective dose may refer to the amount of the antibodies or antigen-binding portions thereof of the present invention against SARS-CoV-2 sufficient to delay or minimize the onset of an infection. An effective dose may also refer to the amount of the antibodies or antigen-binding portions thereof of the present invention against SARS-CoV-2 that provides a therapeutic benefit in the treatment or management of an infection. Further, an effective dose is the amount of the antibodies or antigen-binding portions thereof of the present invention against SARS-CoV-2, or in combination with other therapies, that provides a therapeutic benefit in the treatment or management of an infection. An effective dose may also be the amount sufficient to enhance a subject's (e.g., a human's) own immune response against subsequent exposure to SARS-CoV-2. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered with the antibodies or antigen-binding portions thereof of the present invention against SARS-CoV-2, or can be administered separately. Either the protein or an expression vector encoding the protein can be administered to produce an immunostimulatory effect.

As used herein, the term “protective immune response” or “protective response” refers to an immune response mediated by the antibodies or antigen-binding portions thereof of the present invention against SARS-CoV-2, which is exhibited by a vertebrate (e.g., a human), which prevents or ameliorates an infection or reduces at least one symptom thereof.

The invention further provides kits, including SARS-CoV-2 antibodies, nucleic acids, agents, drugs and pharmaceutical formulations, packaged into suitable packaging material, optionally in combination with instructions for using the kit components, e.g., instructions for performing a method of detection of the invention. In one non-limiting examples, the antibody can be detected by an immunoassay selected from radioimmunoassay, enzyme-linked immunosorbent assay (ELISA), sandwich assays, Western blot, immunoprecipitation, immunohistochemistry, immunofluorescence, antibody microarray, dot blotting, and fluorescence-activated cell sorting (FACS).

In one embodiment, a kit includes a SARS-CoV-2 antibody, subsequence or fragment and instructions for detecting SARS-CoV-2. In another embodiment, a kit includes a SARS-CoV-2 antibody, subsequence or fragment and instructions for treating a subject in need of treatment (e.g., a subject having a disease, disorder, pathology, or condition amendable or that may respond to treatment or therapy) with the SARS-CoV-2 antibody, subsequence or fragment.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, a diagnostic or treatment method of the invention. Instructions can therefore include instructions for practicing any of the methods of the invention described herein. Thus, in various embodiments, a kit includes a label or packaging insert including instructions for practicing a method of the invention in solution, in vitro, in vivo, or ex vivo. In one aspect, the instructions include administering or delivering the SARS-CoV-2 antibody, subsequence or binding fragment thereof into a subject locally, regionally or systemically in a treatment or therapeutic method of the invention.

Instructions may additionally include indications of a satisfactory clinical endpoint or any adverse symptoms or complications that may occur. Instructions may further include storage information, expiration date, or any information required by regulatory agencies such as the Food and Drug Administration for use in a human subject.

The instructions may be on “printed matter”, e.g., on paper or cardboard within the kit, on a label affixed to the kit or packaging material or attached to a vial or tube containing a component of the kit. Instructions may comprise audio or video medium and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include a buffering agent, a preservative, or a stabilizing agent. The kit can also include control components for assaying for activity, e.g., a control sample or a standard. Each component of the kit can be enclosed within an individual container or in a mixture and all of the various containers can be within single or multiple packages.

In accordance with the invention further provided are cell-free (e.g., in solution, in solid phase) and cell-based (e.g., in vitro or in vivo) methods of screening, detecting and identifying SARS-CoV-2. The methods can be performed in solution, in vitro using a biological material or sample, and in vivo, for example, a sample of cells from an animal. In one embodiment, a method includes contacting a biological material or sample with an antibody that binds to SARS-CoV-2 under conditions allowing binding of the antibody SARS-CoV-2; and assaying for binding of the antibody to SARS-CoV-2. The binding of the antibody to SARS-CoV-2 detects the presence of SARS-CoV-2. In one aspect, SARS-CoV-2 is present on a cell or tissue. In another aspect, the biological material or sample is obtained from a mammalian subject.

As used herein, the term “contacting” when used in reference to a composition such as a protein (e.g., SARS-CoV-2 antibody), material, sample, or treatment, means a direct or indirect interaction between the composition (e.g., SARS-CoV-2 antibody) and the other referenced entity. A particular example of direct interaction is binding. A particular example of an indirect interaction is where the composition acts upon an intermediary molecule, which in turn acts upon the referenced entity. Thus, for example, contacting a cell (e.g., a lymphocyte) with the SARS-CoV-2 antibody includes allowing the antibody to bind to the cell (e.g., through binding to SARS-CoV-2), or allowing the antibody to act upon an intermediary that in turn acts upon the cell.

As used herein, the terms “assaying” and “measuring” and grammatical variations thereof are used interchangeably herein and refer to either qualitative or quantitative determinations, or both qualitative and quantitative determinations. When the terms are used in reference to binding, any means of assessing the relative amount, affinity or specificity of binding is contemplated, including the various methods set forth herein and known in the art.

For example, SARS-CoV-2 antibody binding to SARS-CoV-2 can be assayed or measured by an ELISA assay.

The practice of the present invention employs, unless otherwise indicated, conventional methods of chemistry, biochemistry, molecular biology, immunology and pharmacology, within the skill of the art to make the antibodies or antigen-binding portions thereof of the present invention against SARS-CoV-2. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

### Example 1: Neutralization of SARS-CoV-2 Pseudovirions

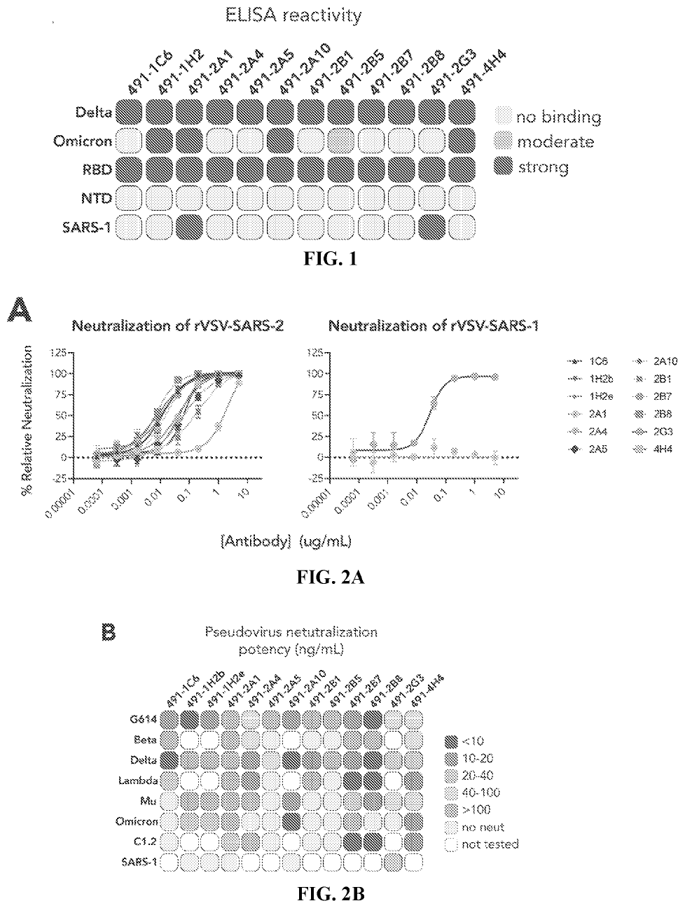

FIG. 1 shows the binding of antibodies disclosed herein to SARS-CoV-2 spike protein variants, domain (receptor-binding, RBD or N-terminal domain, NTD) or SARS-1 spike protein coated on ELISA microtiter plates.

Pre-titrated amounts of rVSV-SARS-CoV-2 (ancestral lineage, G614 or indicated variant, i.e., Beta or C1.2) or rVSV-SARS-1 was incubated with serially diluted purified monoclonal antibody at 37 C for 1 hr before addition to confluent Vero monolayers in 96-well plates. Infection proceeded for 12-16 hrs at 37° C. in 5% C02 before cells were fixed in 4% paraformaldehyde and stained with 10 ug/mL Hoescht. Cells were imaged using a CellInsight CX5 imager and infection was quantitated by automated enumeration of total cells and those expressing GFP. Infection was normalized to the average number of cells infected with the indicated pseudovirus incubated with PBS rather than monoclonal antibody. (FIG. 2A) Data are presented as the relative neutralization for each concentration of antibody. (FIG. 2B) Neutralization IC50s (displayed as ng/mL) were calculated using non-linear regression in GraphPad Prism 9.0.

### (3) Antibody Sequences:

Heavy Chain → 491-1C6 (LJI_0491-30).1

| | | |
|---|---|---|
| CRDH1 | SEQ ID NO: 1 | GGSISRSSHY |
| CRDH2 | SEQ ID NO: 2 | IYYSGTT |
| CRDH3 | SEQ ID NO: 3 | ARRRAGAALPGVEDY |

AA: SEQ ID NO: 4
QVQLVQSGPGLVKPSETLSLTCTVSGGSISRSSHYWGWIRQPPGKGLEWIGSIYYSGT
TYYNPSLKSRVTISVDTSKNQFSLKLNSVTAADTAVYFCARRRAGAALPGVEDYWG
QGTLVTVS
Nt: SEQ ID NO: 5
CAGGTCCAGCTGGTACAGTCTGGCCCAGGACTGGTGAAGCCTTCGGAGACCCTGT
CCCTCACCTGCACTGTCTCTGGTGGCTCCATCAGCAGAAGTAGTCACTACTGGGG
CTGGATCCGCCAGCCCCCAGGGAAGGGGCTGGAGTGGATTGGGAGTATCTATTAT
AGTGGGACCACCTACTACAATCCGTCCCTCAAGAGTCGAGTCACCATATCCGTAG
ACACGTCCAAGAACCAGTTCTCCCTGAAGCTGAACTCTGTGACCGCCGCAGACAC
GGCTGTGTATTTCTGTGCGAGACGAAGGGCTGGAGCAGCTCTTCCGGGGGTGGAG
GACTACTGGGGCCAGGGAACCCTGGTCACCGTCTCC
Light Chain → 491-1C6 (LJI_0491-30).1

| | | |
|---|---|---|
| CDRL1 | SEQ ID NO: 6 | SGSIASNY |
| CDRL2 | SEQ ID NO: 7 | EDN |
| CDRL3 | SEQ ID NO: 8 | QSYDSSSQEV |

AA: SEQ ID NO: 9
RPVLTQPHSVSESPGKTVTISCTRSSGSIASNYVQWFQLRPGSAPTTVIYEDNQRLSGV
PDRFSGSIDSSSNSASLTISGLKTEDEAEYYCQSYDSSSQEVFGGGTKLTVL
Nt: SEQ ID NO: 10
CGGCCCGTGCTGACTCAGCCCCACTCTGTGTCGGAGTCTCCGGGGAAGACGGTAA
CCATCTCCTGCACCCGCAGCAGTGGCAGCATTGCCAGCAACTATGTGCAGTGGTT
CCAGCTGCGCCCGGGCAGTGCCCCCACCACTGTGATCTATGAGGATAACCAAAGA
CTCTCTGGGGTCCCTGATCGGTTCTCTGGCTCCATCGACAGCTCCTCCAACTCTGC
CTCCCTCACCATCTCTGGACTGAAGACTGAGGACGAGGCTGAGTACTACTGTCAG
TCTTATGATAGCAGCAGTCAGGAGGTATTCGGCGGAGGGACCAAGCTGACCGTCC
TA
Heavy Chain → 491-1C6 (LJI_0491-30).2

| | | |
|---|---|---|
| CRDH1 | SEQ ID NO: 11 | GGSISRSSHY |
| CRDH2 | SEQ ID NO: 12 | IYYSGTT |
| CRDH3 | SEQ ID NO: 13 | ARRRAGAALPGVEDY |

AA: SEQ ID NO: 14
QVQLQQLGPGLVKPSETLSLTCTVFGGSISRSSHYWGWIQPPGKGLEWIGSIYYSGT
TYYNPSLKSRVTISVDTSKNQFSLKLNSVTAADTAVYFCARRRAGAALPGVEDYWG
QGTLVTVS
Nt:
SEQ ID NO: 15
CAGGTGCAGCTGCAGCAGTTGGGCCCAGGACTGGTGAAGCCTTCGGAGACCCTGT
CCCTCACCTGCACTGTTTTTGGTGGCTCCATCAGCAGAAGTAGTCACTACTGGGG
CTGGATCCGCCAGCCCCCAGGGAAGGGGCTGGAGTGGATTGGGAGTATTTATTAT
AGTGGGACCACCTACTACAATCCGTCCCTCAAGAGTCGAGTCACCATATCCGTAG
ACACGTCCAAGAACCAGTTTTCCCTGAAGCTGAACTCTGTGACCGCCGCAGACAC
GGCTGTGTATTTCTGTGCGAGACGAAGGGCTGGAGCAGCTCTTCCGGGGGTGGAG
GACTACTGGGGCCAGGGAACCCTGGTCACCGTCTCC
Light Chain → 491-1C6 (LJI_0491-30).2

| | | |
|---|---|---|
| CDRL1 | SEQ ID NO: 16 | SGSIASNY |
| CDRL2 | SEQ ID NO: 17 | EDN |
| CDRL3 | SEQ ID NO: 18 | QSYDSSSQEV |

AA: SEQ ID NO: 19
NFMLTQPHSVSESPGKTVTISCTRSSGSIASNYVQWYQLRPGSAPTTVIYEDNQRPSG
VPDRFFGSIDSSSNSASLTISGLKTEDEAEYYCQSYDSSSQEVFGGGTKLTVL
Nt: SEQ ID NO: 20
AATTTTATGtTGAcTCAGCCGCACTCTGTGTCGGAGTCTCCGGGGAAGACGGTAAC
CATCTCCTGCACCCGCAGCAGTGGCAGCATTGCCAGCAATTATGTGCAGTGGTAC
CAGCTGCGCCCGGGCAGTGCCCCCACCACTGTGATCTATGAGGATAACCAAAGAC
CCTCTGGGGTCCCTGATCGGTTCTTTGGCTCCATCGACAGCTCCTCCAACTCTGCC
TCCCTCACCATCTCTGGACTGAAGACTGAGGACGAGGCTGAGTACTACTGTCAGT
CTTATGATAGCAGCAGTCAGGAGGTATTCGGCGGAGGGACCAAGCTGACCGTCCT
A
Heavy Chain → 491-1H2b (LJI_0491-86b)

| | | |
|---|---|---|
| CRDH1 | SEQ ID NO: 21 | GFTFDDYA |
| CRDH2 | SEQ ID NO: 22 | ITWNSGTI |
| CRDH3 | SEQ ID NO: 23 | AKDSGRKLLWGREDYYMGV |

AA: SEQ ID NO: 24
QVQLQQSGGGLVQPGRSLRLSCAASGFTFDDYAMHWVRQAPGKGLEWVSGITWNS
GTIGYADSVKGRFTISRDNAKNSLYLQMRSLRAEDTALYYCAKDSGRKLLWGRED
YYMGVWGKGTTVTVS
Nt: SEQ ID NO: 25
CAGGTACAGCTGCAGCAGTCAGGGGGAGGCTTGGTACAGCCTGGCAGGTCCCTG
AGACTCTCCTGTGCAGCCTCTGGATTCACCTTTGATGATTATGCCATGCACTGGGT
CCGGCAAGCTCCAGGGAAGGGCCTGGAGTGGGTCTCAGGTATTACTTGGAATAGT
GGTACCATAGGCTATGCGGACTCTGTGAAGGGCCGATTCACCATCTCCAGAGACA
ACGCCAAGAACTCCCTCTATCTGCAAATGAGAAGTCTTAGAGCTGAGGACACGGC
CTTGTATTACTGTGCAAAAGATTCCGGGCGGAAACTGCTTTGGGGGCGTGAGGAC
TACTACATGGGCGTCTGGGGCAAAGGGACCACGGTCACCGTCTCC
Heavy Chain → 491-1H2e (LJI_0491-86e).1

| | | |
|---|---|---|
| CRDH1 | SEQ ID NO: 26 | GFTFDDYA |
| CRDH2 | SEQ ID NO: 27 | ITWNSGTI |
| CRDH3 | SEQ ID NO: 28 | AKDSGRKQLWGREDYYMGV |

AA: SEQ ID NO: 29
NO:QVQLVQSGGGLVQPGRSLRLSCAASGFTFDDYAMHWVRQAPGKGLEWVSGIT
WNSGTIGYADTVKGRFTISRDNAKNSLYLQMRSLRAEDTALYYCAKDSGRKQLWGR
EDYYMGVWGKGTTVTVS
Nt: SEQ ID NO: 30
NO:CAGGTCCAGCTGGTACAGTCTGGTGGAGGCTTGGTACAGCCTGGCAGGTCCC
TGAGACTCTCCTGTGCAGCCTCTGGATTCACCTTTGATGATTATGCCATGCACTGG
GTCCGGCAAGCTCCAGGGAAGGGCCTGGAGTGGGTCTCAGGTATTACTTGGAATA
GTGGTACCATAGGCTATGCGGACACTGTGAAGGGCCGATTCACCATCTCCAGAGA
CAACGCCAAGAACTCCCTCTATCTGCAAATGAGAAGTCTTAGAGCTGAGGACACG
GCCTTGTATTACTGTGCAAAAGATTCCGGGCGGAAACAGCTTTGGGGGCGTGAGG
ACTACTACATGGGCGTCTGGGGCAAAGGGACCACGGTCACCGTCTCC
Light Chain → 491-1H2 (LJI_0491-86)

| | | |
|---|---|---|
| CDRL1 | SEQ ID NO: 36 | NIGSKS |
| CDRL2 | SEQ ID NO: 37 | YDS |
| CDRL3 | SEQ ID NO: 38 | QVWDSSSDHVV |

AA: SEQ ID NO: 39
SYELTQPPSVSVAPGKTARITCGGSNIGSKSVHWYQQKPGQAPVLVIYYDSDRPSGIP
ERFSGSNSGNTATLTISRVEAGDEADYYCQVWDSSSDHVVFGGGTKLTVL
Nt: SEQ ID NO: 40
TCCTATGAGCTGACTCAGCCACCCTCTGTGTCAGTGGCCCCAGGAAAGACGGCCA
GGATTACTTGTGGGGGAAGCAACATTGGGAGTAAAAGTGTGCACTGGTACCAGC
AGAAGCCAGGCCAGGCCCCTGTGCTGGTCATCTATTATGATAGCGACCGGCCCTC
AGGGATCCCTGAGCGATTCTCTGGCTCCAACTCTGGGAACACGGCCACCCTGACC
ATCAGCAGGGTCGAAGCCGGGGATGAGGCCGACTATTACTGTCAGGTGTGGGAT
AGTAGCAGTGATCATGTGGTATTCGGCGGAGGGACCAAGCTGACCGTCCTA
Heavy Chain → 491-1H2e (LJI_0491-86e).2

| | | |
|---|---|---|
| CRDH1 | SEQ ID NO: 31 | GFTFDDYA |
| CRDH2 | SEQ ID NO: 32 | ITWNSGTI |
| CRDH3 | SEQ ID NO: 33 | AKDSGRKQLWGREDYYMGV |

AA: SEQ ID NO: 34
EVQLVEFGGGLVQPGRSLRLSCAASGFTFDDYAMHWVRQAPGKGLEWVSGITWNS
GTIGYADSVKGRFTISRDNAKNSLYLQMRSLRAEDTALYYCAKDSGRKQLWGRED
YYMGVWGKGTTVTVS
Nt: SEQ ID NO: 35
GAAGTGCAGCTGGTGGAGTCTGGGGGAGGCTTGGTACAGCCTGGCAGGTCCCTG
AGACTTTCCTGTGCAGCCTTTGGATTCACCTTTGATGATTATGCCATGCACTGGGT
CCGGCAAGCTCCAGGGAAGGGCCTGGAGTGGGTTTCAGGTATTACTTGGAATAGT
GGTACCATAGGCTATGCGGACTCTGTGAAGGGCCGATTCACCATCTCCAGAGACA
ACGCCAAGAACTCCCTCTATCTGCAAATGAGAAGTCTTAGAGCTGAGGACACGGC
CTTGTATTACTGTGCAAAAGATTCCGGGCGGAAACAGCTTTGGGGGCGTGAGGAC
TACTACATGGGCGTCTGGGGCAAAGGGACCACGGTCACCGTCTCC
Light Chain → 491-1H2 (LJI_0491-86)

| | | |
|---|---|---|
| CDRL1 | SEQ ID NO: 36 | NIGSKS |
| CDRL2 | SEQ ID NO: 37 | YDS |
| CDRL3 | SEQ ID NO: 38 | QVWDSSSDHVV |

AA: SEQ ID NO: 39
SYELTQPPSVSVAPGKTARITCGGSNIGSKSVHWYQQKPGQAPVLVIYYDSDRPSGIP
ERFSGSNSGNTATLTISRVEAGDEADYYCQVWDSSSDHVVFGGGTKLTVL
Nt: SEQ ID NO: 40
TCCTATGAGCTGACTCAGCCACCCTCTGTGTCAGTGGCCCCAGGAAAGACGGCCA
GGATTACTTGTGGGGGAAGCAACATTGGGAGTAAAAGTGTGCACTGGTACCAGC
AGAAGCCAGGCCAGGCCCCTGTGCTGGTCATCTATTATGATAGCGACCGGCCCTC
AGGGATCCCTGAGCGATTCTCTGGCTCCAACTCTGGGAACACGGCCACCCTGACC
ATCAGCAGGGTCGAAGCCGGGGATGAGGCCGACTATTACTGTCAGGTGTGGGAT
AGTAGCAGTGATCATGTGGTATTCGGCGGAGGGACCAAGCTGACCGTCCTA
Heavy Chain → 491-2A1 (LJI_0491-97).1

| | | |
|---|---|---|
| CRDH1 | SEQ ID NO: 41 | GYRFTRHW |
| CRDH2 | SEQ ID NO: 42 | IYPGDSDT |
| CRDH3 | SEQ ID NO: 43 | ARHVYCSSSTCYTTEGGWFDA |

AA: SEQ ID NO: 44
QVQLVESGAEVKKPGESLKISCKGSGYRFTRHWIGWVRQMPGKGLEWMGIIYPGD
SDTRYSPSFQGQVTISADKSIGTAYVQWSSLKASDTAMYYCARHVYCSSSTCYTTE
GGWFDAWGQGTLVTVS
Nt: SEQ ID NO: 45
CAGGTACAGCTCGTGGAGTCTGGAGCAGAGGTGAAAAAGCCGGGGGAGTCTCTG
AAGATCTCCTGTAAGGGTTCTGGATACAGGTTTACCAGACACTGGATCGGCTGGG
TGCGCCAGATGCCCGGGAAAGGCCTGGAGTGGATGGGGATCATCTATCCTGGTG
ACTCTGATACCAGATACAGCCCGTCCTTCCAAGGCCAGGTCACCATCTCAGCCGA
CAAGTCCATCGGCACCGCATATGTGCAGTGGAGCAGCCTGAAGGCCTCGGACAC
CGCCATGTATTATTGTGCGAGACATGTATATTGTAGTAGTAGCACCTGCTACACG
ACCGAGGGGGGGTGGTTCGACGCCTGGGGCCAGGGAACCCTGGTCACCGTCTCC
Light Chain → 491-2A1 (LJI_0491-97)

| | | |
|---|---|---|
| CDRL1 | SEQ ID NO: 51 | SSNIGSNT |
| CDRL2 | SEQ ID NO: 52 | SNN |
| CDRL3 | SEQ ID NO: 53 | AAWDDSLNGVV |

AA: SEQ ID NO: 54
QSVLTQPPSASGTPGQRVTISCSGSSSNIGSNTVNWYQQLPGTAPKLLIYSNNQRPSG
VPDRFSDSKSGTSASLAISGLQSEDEADYYCAAWDDSLNGVVFGGGTKLTVL
Nt: SEQ ID NO: 55
CAGTCTGTTCTGACTCAGCCACCCTCAGCGTCTGGGACCCCCGGGCAGAGGGTCA
CCATCTCTTGTTCTGGAAGCAGCTCCAACATCGGAAGTAATACTGTAAACTGGTA
CCAGCAGCTCCCAGGAACGGCCCCCAAACTCCTCATCTATAGTAATAATCAGCGG
CCCTCAGGGGTCCCTGACCGATTCTCTGACTCCAAGTCTGGCACCTCAGCCTCCCT
GGCCATCAGTGGGCTCCAGTCTGAGGATGAGGCTGATTATTACTGTGCAGCCTGG
GATGACAGCCTGAATGGTGTGGTATTCGGCGGAGGGACCAAGCTTACCGTCCTA
Heavy Chain → 491-2A1 (LJI_0491-97).2

| | | |
|---|---|---|
| CRDH1 | SEQ ID NO: 46 | GYRFTRHW |
| CRDH2 | SEQ ID NO: 47 | IYPGDSDT |
| CRDH3 | SEQ ID NO: 48 | ARHVYCSSSTCYTTEGGWFDA |

AA: SEQ ID NO: 49
EVQLVQSGAEVKKPGESLKISCKGSGYRFTRHWIGWVRQMPGKGLEWMGIIYPGD
SDTRYSPSFQGQVTISADKSIGTAYVQWSSLKASDTAMYYCARHVYCSSSTCYTTE
GGWFDAWGQGTLVTVS
Nt: SEQ ID NO: 50
GAGGTGCAGCTGGTGCAGTCTGGAGCAGAGGTGAAAAAGCCGGGGGAGTCTCTG
AAGATCTCCTGTAAGGGTTCTGGATACAGGTTTACCAGACACTGGATCGGCTGGG
TGCGCCAGATGCCCGGGAAAGGCCTGGAGTGGATGGGGATCATCTATCCTGGTG
ACTCTGATACCAGATACAGCCCGTCCTTCCAAGGCCAGGTCACCATCTCAGCCGA
CAAGTCCATCGGCACCGCATATGTGCAGTGGAGCAGCCTGAAGGCCTCGGACAC
CGCCATGTATTATTGTGCGAGACATGTATATTGTAGTAGTAGCACCTGCTACACG
ACCGAGGGGGGGTGGTTCGACGCCTGGGGCCAGGGAACCCTGGTCACCGTCTCC
Light Chain → 491-2A1 (LJI_0491-97)

| | | |
|---|---|---|
| CDRL1 | SEQ ID NO: 51 | SSNIGSNT |
| CDRL2 | SEQ ID NO: 52 | SNN |
| CDRL3 | SEQ ID NO: 53 | AAWDDSLNGVV |

AA: SEQ ID NO: 54
QSVLTQPPSASGTPGQRVTISCSGSSSNIGSNTVNWYQQLPGTAPKLLIYSNNQRPSG
VPDRFSDSKSGTSASLAISGLQSEDEADYYCAAWDDSLNGVVFGGGTKLTVL
Nt: SEQ ID NO: 55
CAGTCTGTTCTGACTCAGCCACCCTCAGCGTCTGGGACCCCCGGGCAGAGGGTCA
CCATCTCTTGTTCTGGAAGCAGCTCCAACATCGGAAGTAATACTGTAAACTGGTA
CCAGCAGCTCCCAGGAACGGCCCCCAAACTCCTCATCTATAGTAATAATCAGCGG
CCCTCAGGGGTCCCTGACCGATTCTCTGACTCCAAGTCTGGCACCTCAGCCTCCCT
GGCCATCAGTGGGCTCCAGTCTGAGGATGAGGCTGATTATTACTGTGCAGCCTGG
GATGACAGCCTGAATGGTGTGGTATTCGGCGGAGGGACCAAGCTTACCGTCCTA
Heavy Chain → 491-2A4 (LJI_0491-100).1

| | | |
|---|---|---|
| CRDH1 | SEQ ID NO: 56 | GITVSKNY |
| CRDH2 | SEQ ID NO: 57 | IYSGGST |
| CRDH3 | SEQ ID NO: 58 | ARDLGDYFDY |

AA: SEQ ID NO: 59
QVQLVQSGGGLIQPGGSLRLSCAASGITVSKNYMNWVRQAPGKGLEWVSVIYSGGS
TFYADSVKGRFTISRDNSKNTLYLQMNSLRAEDTAVYYCARDLGDYFDYWGQGTL
VTVS
Nt: SEQ ID NO: 60
CAGGTCCAGCTGGTACAGTCTGGAGGAGGCTTGATCCAGCCTGGGGGGTCCCTGA
GACTCTCCTGTGCAGCCTCTGGGATCACCGTCAGTAAAAACTACATGAACTGGGT
CCGCCAGGCTCCAGGGAAGGGGCTGGAGTGGGTCTCAGTTATTTATAGCGGTGGT
AGCACATTCTACGCAGACTCCGTGAAGGGCCGATTCACCATCTCCAGAGACAATT
CCAAGAACACGCTGTATCTGCAAATGAACAGCCTGAGAGCCGAGGACACGGCCG
TGTATTACTGTGCGAGAGATCTCGGTGACTACTTTGACTACTGGGGCCAGGGAAC
CCTGGTCACCGTCTCC
Light Chain → 491-2A4 (LJI_0491-100).1

| | | |
|---|---|---|
| CDRL1 | SEQ ID NO: 61 | QGISSY |
| CDRL2 | SEQ ID NO: 62 | AAS |
| CDRL3 | SEQ ID NO: 63 | QQLNSYPPNT |

AA: SEQ ID NO: 64
DIVMTQTPSFLSASVGDRVTITCRASQGISSYLAWYQQKPGKAPKLLIYAASTLQSGV
PSRFSGSGSGTEFTLTISSLQPEDFATYYCQQLNSYPPNTFGQGTKLEIK
Nt: SEQ ID NO: 65
GATATTGTGATGACCCAGACTCCATCCTTCCTGTCTGCATCTGTAGGAGACAGAG
TCACCATCACTTGCCGGGCCAGTCAGGGCATTAGCAGTTATTTAGCCTGGTATCA
GCAAAAACCAGGGAAAGCCCCTAAGCTCCTGATCTATGCTGCATCCACTTTGCAA
AGTGGGGTCCCATCAAGGTTCAGCGGCAGTGGATCTGGGACAGAATTCACTCTCA
CAATCAGCAGCCTGCAGCCTGAAGATTTTGCAACTTATTACTGTCAACAGCTTAA
TAGTTACCCTCCGAACACTTTTGGCCAGGGGACCAAGCTGGAGATCAAA
Heavy Chain → 491-2A4 (LJI_0491-100).2

| | | |
|---|---|---|
| CRDH1 | SEQ ID NO: 66 | GITVSKNY |
| CRDH2 | SEQ ID NO: 67 | IYSGGST |
| CRDH3 | SEQ ID NO: 68 | ARDLGDYFDY |

AA: SEQ ID NO: 69
EVQLVESGGGLIQPGGSLRLSCAASGITVSKNYMNWVRQAPGKGLEWVSVIYSGGS
TFYADSVKGRFTISRDNSKNTLYLQMNSLRAEDTAVYYCARDLGDYFDYWGQGTL
VTVS
Nt: SEQ ID NO: 70
GAGGTGCAGCTGGTGGAGTCTGGAGGAGGCTTGATCCAGCCTGGGGGGTCCCTG
AGACTCTCCTGTGCAGCCTCTGGGATCACCGTCAGTAAAAACTACATGAACTGGG
TCCGCCAGGCTCCAGGGAAGGGGCTGGAGTGGGTCTCAGTTATTTATAGCGGTGG
TAGCACATTCTACGCAGACTCCGTGAAGGGCCGATTCACCATCTCCAGAGACAAT
TCCAAGAACACGCTGTATCTGCAAATGAACAGCCTGAGAGCCGAGGACACGGCC
GTGTATTACTGTGCGAGAGATCTCGGTGACTACTTTGACTACTGGGGCCAGGGAA
CCCTGGTCACCGTCTCC
Light Chain → 491-2A4 (LJI_0491-100).2

| | | |
|---|---|---|
| CDRL1 | SEQ ID NO: 71 | QGISSY |
| CDRL2 | SEQ ID NO: 72 | AAS |
| CDRL3 | SEQ ID NO: 73 | QQLNSYPPNT |

AA: SEQ ID NO: 74
DIQLTQSPSFLSASVGDRVTITCRASQGISSYLAWYQQKPGKAPKLLIYAASTLQSGV
PSRFSGSGSGTEFTLTISSLQPEDFATYYCQQLNSYPPNTFGQGTKLEIK
Nt: SEQ ID NO: 75
GACATCCAGTTGACCCAGTCTCCATCCTTCCTGTCTGCATCTGTAGGAGACAGAG
TCACCATCACTTGCCGGGCCAGTCAGGGCATTAGCAGTTATTTAGCCTGGTATCA
GCAAAAACCAGGGAAAGCCCCTAAGCTCCTGATCTATGCTGCATCCACTTTGCAA
AGTGGGGTCCCATCAAGGTTCAGCGGCAGTGGATCTGGGACAGAATTCACTCTCA
CAATCAGCAGCCTGCAGCCTGAAGATTTTGCAACTTATTACTGTCAACAGCTTAA
TAGTTACCCTCCGAACACTTTTGGCCAGGGGACCAAGCTGGAGATCAAA
Heavy Chain → 491-2A5 (LJI_0491-101).1

| | | |
|---|---|---|
| CRDH1 | SEQ ID NO: 76 | GYTFTSYD |
| CRDH2 | SEQ ID NO: 77 | MNPNSGNT |
| CRDH3 | SEQ ID NO: 78 | ARGRYFDWLKGIEDYEEDRVGNY |

AA: SEQ ID NO: 79
EVQLLESGAEVKKPGASVKVSCKASGYTFTSYDINWVRQATGQGLEWMGWMNPN
SGNTGYAQKFQGRVTMTRNTSISTAYMELSSLRSEDTAVYYCARGRYFDWLKGIE
DYEEDRVGNYWGQGTLVTVS
Nt. SEQ ID NO: 80
GAGGTGCAGCTGTTGGAGTCTGGGGCTGAGGTGAAGAAGCCTGGGGCCTCAGTG
AAGGTCTCCTGCAAGGCTTCTGGATACACCTTCACCAGTTATGATATCAACTGGG
TGCGACAGGCCACTGGACAAGGGCTTGAGTGGATGGGATGGATGAACCCTAACA
GTGGTAACACAGGCTATGCACAGAAGTTCCAGGGCAGAGTCACCATGACCAGGA
ACACCTCCATAAGCACAGCCTACATGGAGCTGAGCAGCCTGAGATCTGAGGACA
CGGCCGTGTATTACTGTGCGAGAGGCAGATATTTTGACTGGTTAAAGGGAATAGA
GGACTACGAAGAAGATAGGGTGGGGAACTACTGGGGCCAGGGAACCCTGGTCAC
CGTCTCC
Light Chain → 491-2A5 (LJI_0491-101).1

| | | |
|---|---|---|
| CDRL1 | SEQ ID NO: 81 | QSVSSY |
| CDRL2 | SEQ ID NO: 82 | DAS |
| CDRL3 | SEQ ID NO: 83 | QQRSNWPPLT |

AA: SEQ ID NO: 84
DIVMTQSPSTLSLSPGERATLSCRASQSVSSYLAWYQQKPGQAPRLLIYDASNRATGI
PARFSGSGSGTDFTLTISSLEPEDFAVYYCQQRSNWPPLTFGGGTKVEIK
Nt: SEQ ID NO: 85
GACATCGTGATGACCCAGTCTCCATCCACCCTGTCTTTGTCTCCAGGGGAAAGAG
CCACCCTCTCCTGCAGGGCCAGTCAGAGTGTTAGCAGCTACTTAGCCTGGTACCA
ACAGAAACCTGGCCAGGCTCCCAGGCTCCTCATCTATGATGCATCCAACAGGGCC
ACTGGCATCCCAGCCAGGTTCAGTGGCAGTGGGTCTGGGACAGACTTCACTCTCA
CCATCAGCAGCCTAGAGCCTGAAGATTTTGCAGTTTATTACTGTCAGCAGCGTAG
CAACTGGCCTCCGCTCACTTTCGGCGGAGGGACCAAGGTGGAGATCAAA
Heavy Chain → 491-2A5 (LJI_0491-101).2

| | | |
|---|---|---|
| CRDH1 | SEQ ID NO: 86 | GYTFTSYD |
| CRDH2 | SEQ ID NO: 87 | MNPNSGNT |
| CRDH3 | SEQ ID NO: 88 | ARGRYFDWLKGIEDYEEDRVGNY |

AA: SEQ ID NO: 89
QVQLVQSGAEVKKPGASVKVSCKASGYTFTSYDINWVRQATGQGLEWMGWMNPN
SGNTGYAQKFQGRVTMTRNTSISTAYMELSSLRSEDTAVYYCARGRYFDWLKGIE
DYEEDRVGNYWGQGTLVTVS
Nt: SEQ ID NO: 90
CAGGTGCAGCTGGTGCAGTCTGGGGCTGAGGTGAAGAAGCCTGGGGCCTCAGTG
AAGGTCTCCTGCAAGGCTTCTGGATACACCTTCACCAGTTATGATATCAACTGGG
TGCGACAGGCCACTGGACAAGGGCTTGAGTGGATGGGATGGATGAACCCTAACA
GTGGTAACACAGGCTATGCACAGAAGTTCCAGGGCAGAGTCACCATGACCAGGA
ACACCTCCATAAGCACAGCCTACATGGAGCTGAGCAGCCTGAGATCTGAGGACA
CGGCCGTGTATTACTGTGCGAGAGGCAGATATTTTGACTGGTTAAAGGGAATAGA
GGACTACGAAGAAGATAGGGTGGGGAACTACTGGGGCCAGGGAACCCTGGTCAC
CGTCTCC
Light Chain → 491-2A5 (LJI_0491-101).2

| | | |
|---|---|---|
| CDRL1 | SEQ ID NO: 91 | QSVSSY |
| CDRL2 | SEQ ID NO: 92 | DAS |
| CDRL3 | SEQ ID NO: 93 | QQRSNWPPLT |

AA: SEQ ID NO: 94
EIVLTQSPATLSLSPGERATLSCRASQSVSSYLAWYQQKPGQAPRLLIYDASNRATGIP
ARFSGSGSGTDFTLTISSLEPEDFAVYYCQQRSNWPPLTFGGGTKVEIK
Nt: SEQ ID NO: 95
GAAATTGTGTTGACACAGTCTCCAGCCACCCTGTCTTTGTCTCCAGGGGAAAGAG
CCACCCTCTCCTGCAGGGCCAGTCAGAGTGTTAGCAGCTACTTAGCCTGGTACCA
ACAGAAACCTGGCCAGGCTCCCAGGCTCCTCATCTATGATGCATCCAACAGGGCC
ACTGGCATCCCAGCCAGGTTCAGTGGCAGTGGGTCTGGGACAGACTTCACTCTCA
CCATCAGCAGCCTAGAGCCTGAAGATTTTGCAGTTTATTACTGTCAGCAGCGTAG
CAACTGGCCTCCGCTCACTTTCGGCGGAGGGACCAAGGTGGAGATCAAA
Heavy Chain → 491-2A10 (LJI_0491-106).1

| | | |
|---|---|---|
| CRDH1 | SEQ ID NO: 96 | EFIVSSNY |
| CRDH2 | SEQ ID NO: 97 | LYSGGTT |
| CRDH3 | SEQ ID NO: 98 | ARDRGPWLHDY |

AA: SEQ ID NO: 99
QVQLVESGGGLIQPGGSLRLSCAASEFIVSSNYMSWVRQAPGKGLEWVSLLYSGGT
TYYADSVKGRFTISRDNSKNTLFLQMNSLRAEDTAMYYCARDRGPWLHDYWGQG
TLVTVS
Nt: SEQ ID NO: 100
CAGGTACAGCTCGTGGAGTCTGGAGGAGGCTTGATCCAGCCTGGGGGGTCCCTGA
GACTCTCCTGTGCAGCCTCTGAGTTCATCGTCAGTAGCAACTACATGAGCTGGGT
CCGCCAGGCTCCAGGGAAGGGGCTGGAGTGGGTCTCACTTCTTTATAGCGGTGGT
ACCACATACTACGCAGACTCCGTGAAGGGCCGATTCACCATCTCCAGAGACAATT
CCAAGAACACACTGTTTCTTCAAATGAACAGCCTGAGAGCCGAAGACACGGCCA
TGTATTACTGTGCGAGAGATAGGGGTCCGTGGTTACATGACTACTGGGGCCAGGG
AACCCTGGTCACCGTCTCC
Light Chain → 491-2A10 (LJI_0491-106).1

| | | |
|---|---|---|
| CDRL1 | SEQ ID NO: 106 | QDINKY |
| CDRL2 | SEQ ID NO: 107 | DAS |
| CDRL3 | SEQ ID NO: 108 | QQYDNLPPT |

AA: SEQ ID NO: 109
DIVMTQSPSSLSASVGDRVTITCQASQDINKYCNWYQQKPGKAPKLLIYDASNLETG
VPSRFSGSGSGTDFTFTINSLQPEDIATYYCQQYDNLPPTFGGGTKVEIK
Nt: SEQ ID NO: 110
GACATCGTGATGACCCAGTCTCCATCCTCCCTGTCTGCATCTGTAGGAGACAGAG
TCACCATCACTTGCCAGGCGAGTCAGGACATTAACAAGTATTGCAATTGGTATCA
GCAGAAACCAGGGAAAGCCCCTAAACTCCTGATCTACGATGCATCCAATTTGGAA
ACAGGGGTCCCATCAAGGTTCAGTGGAAGTGGATCTGGGACAGACTTTACTTTCA
CCATCAACAGCCTGCAGCCTGAAGATATTGCAACATATTACTGTCAACAATATGA
TAATCTCCCTCCCACTTTCGGCGGAGGGACCAAGGTGGAAATCAAA
Heavy Chain → 491-2A10 (LJI_0491-106).2

| | | |
|---|---|---|
| CRDH1 | SEQ ID NO: 101 | EFIVSSNY |
| CRDH2 | SEQ ID NO: 102 | LYSGGTT |
| CRDH3 | SEQ ID NO: 103 | ARDRGPWLHDY |

AA: SEQ ID NO: 104
EVQLVESGGGLIQPGGSLRLSCAASEFIVSSNYMRWVRQAPGKGLEWVSLLYSGGT
TYYADSVKGRFTISRDNSKNTLFLQMNSLRAEDTAMYYCARDRGPWLHDYWGQG
TLVTVS
Nt: SEQ ID NO: 105
GAGGTGCAGCTGGTGGAGTCTGGAGGAGGCTTGATCCAGCCTGGGGGGTCCCTG
AGACTTTCCTGTGCAGCCTCTGAGTTCATCGTCAGTAGCAACTACATGAGATGGG
TCCGCCAGGCTCCAGGGAAGGGGCTGGAGTGGGTCTCACTTCTTTATAGCGGTGG
TACCACATACTACGCAGACTCCGTGAAGGGCCGATTCACCATCTCCAGAGACAAT
TCCAAGAACACACTGTTTCTTCAAATGAACAGCCTGAGAGCCGAAGACACGGCC
ATGTATTACTGTGCGAGAGATAGGGGTCCGTGGTTACATGACTACTGGGGCCAGG
GAACCCTGGTCACCGTCTCC
Light Chain → 491-2A10 (LJI_0491-106).1

| | | |
|---|---|---|
| CDRL1 | SEQ ID NO: 106 | QDINKY |
| CDRL2 | SEQ ID NO: 107 | DAS |
| CDRL3 | SEQ ID NO: 108 | QQYDNLPPT |

AA: SEQ ID NO: 109
DIVMTQSPSSLSASVGDRVTITCQASQDINKYCNWYQQKPGKAPKLLIYDASNLETG
VPSRFSGSGSGTDFTFTINSLQPEDIATYYCQQYDNLPPTFGGGTKVEIK
Nt: SEQ ID NO: 110
GACATCGTGATGACCCAGTCTCCATCCTCCCTGTCTGCATCTGTAGGAGACAGAG
TCACCATCACTTGCCAGGCGAGTCAGGACATTAACAAGTATTGCAATTGGTATCA
GCAGAAACCAGGGAAAGCCCCTAAACTCCTGATCTACGATGCATCCAATTTGGAA
ACAGGGGTCCCATCAAGGTTCAGTGGAAGTGGATCTGGGACAGACTTTACTTTCA
CCATCAACAGCCTGCAGCCTGAAGATATTGCAACATATTACTGTCAACAATATGA
TAATCTCCCTCCCACTTTCGGCGGAGGGACCAAGGTGGAAATCAAA
Heavy Chain → 491-2B1 (LJI_0491-109).1

| | | |
|---|---|---|
| CRDH1 | SEQ ID NO: 111 | GYTFTSYD |
| CRDH2 | SEQ ID NO: 112 | MNPNSGNT |
| CRDH3 | SEQ ID NO: 113 | ARGRYFDWLKGIEDYEEDRVGNY |

AA: SEQ ID NO: 114
EVQLVQSGAEVKKPGASVKVSCKASGYTFTSYDINWVRQATGQGLEWMGWMNPN
SGNTGYAQKFQGRVTMTRNTSISTAYMELSSLRSEDTAVYYCARGRYFDWLKGIE
DYEEDRVGNYWGQGTLVTVS
Nt: SEQ ID NO: 115
GAAGTGCAGCTGGTGCAGTCTGGGGCTGAGGTGAAGAAGCCTGGGGCCTCAGTG
AAGGTCTCCTGCAAGGCTTCTGGATACACCTTCACCAGTTATGATATCAACTGGG
TGCGACAGGCCACTGGACAAGGGCTTGAGTGGATGGGATGGATGAACCCTAACA
GTGGTAACACAGGCTATGCACAGAAGTTCCAGGGCAGAGTCACCATGACCAGGA
ACACCTCCATAAGCACAGCCTACATGGAGCTGAGCAGCCTGAGATCTGAGGACA
CGGCCGTGTATTACTGTGCGAGAGGCAGATATTTTGACTGGTTAAAGGGAATAGA
GGACTACGAAGAAGATAGGGTGGGGAACTACTGGGGCCAGGGAACCCTGGTCAC
CGTCTCC
Light Chain → 491-2B1 (LJI_0491-109).1

| | | |
|---|---|---|
| CDRL1 | SEQ ID NO: 116 | QSVSSY |
| CDRL2 | SEQ ID NO: 117 | DAS |
| CDRL3 | SEQ ID NO: 118 | QQRSNWPPLT |

AA: SEQ ID NO: 119
DIVLTQSPATLSLSPGERATLSCRASQSVSSYLAWYQQKPGQAPRLLIYDASNRATGIP
ARFSGSGSGTDFTLTISSLEPEDFAVYYCQQRSNWPPLTFGGGTKVEIK
Nt: SEQ ID NO: 120
GACATTGTGCTGACCCAGTCTCCAGCCACCCTGTCTTTGTCTCCAGGGGAAAGAG
CCACCCTCTCCTGCAGGGCCAGTCAGAGTGTTAGCAGCTACTTAGCCTGGTACCA
ACAGAAACCTGGCCAGGCTCCCAGGCTCCTCATCTATGATGCATCCAACAGGGCC
ACTGGCATCCCAGCCAGGTTCAGTGGCAGTGGGTCTGGGACAGACTTCACTCTCA
CCATCAGCAGCCTAGAGCCTGAAGATTTTGCAGTTTATTACTGTCAGCAGCGTAG
CAACTGGCCTCCGCTCACTTTCGGCGGAGGGACCAAGGTGGAGATCAAA
Heavy Chain → 491-2B1 (LJI_0491-109).2

| | | |
|---|---|---|
| CRDH1 | SEQ ID NO: 121 | GYTFTSYD |
| CRDH2 | SEQ ID NO: 122 | MNPNSGNT |
| CRDH3 | SEQ ID NO: 123 | ARGRYFDWLKGIEDYVEDRVGNY |

AA: SEQ ID NO: 124
QVQLVQSGAEVKKPGASVKVSCKASGYTFTSYDINWVRQATGQGLEWMGWMNPN
SGNTGYAQKFQGRVTMTRNTSISTAYMELSSLRFEDTAVYYCARGRYFDWLKGIE
DYVEDRVGNYWGQGTLVTVS
Nt: SEQ ID NO: 125
CAGGTGCAGCTGGTGCAGTCTGGGGCTGAGGTGAAGAAGCCTGGGGCCTCAGTG
AAGGTCTCCTGCAAGGCTTCTGGATACACCTTCACCAGTTATGATATCAACTGGG
TGCGACAGGCCACTGGACAAGGGCTTGAGTGGATGGGATGGATGAACCCTAACA
GTGGTAACACAGGCTATGCACAGAAGTTCCAGGGCAGAGTCACCATGACCAGGA
ACACCTCCATAAGCACAGCCTACATGGAGCTGAGCAGCCTGAGATTTGAGGACA
CGGCCGTGTATTACTGTGCGAGAGGCAGATATTTTGACTGGTTAAAGGGAATAGA
GGACTACGTAGAAGATAGGGTGGGGAACTACTGGGGCCAGGGAACCCTGGTCAC
CGTCTCC
Light Chain → 491-2B1 (LJI_0491-109).2

| | | |
|---|---|---|
| CDRL1 | SEQ ID NO: 126 | QSVSSY |
| CDRL2 | SEQ ID NO: 127 | DAS |
| CDRL3 | SEQ ID NO: 128 | QQRSNWPPLT |

AA: SEQ ID NO: 129
EIVLTQSPATLSLSPGERATLSCRASQSVSSYLAWYQQKPGQAPRLLIYDASNRATGIP
ARFSGSGSGTDFTLTISSLEPEDFAVYYCQQRSNWPPLTFGGGTKVEIK
Nt: SEQ ID NO: 130
GAAATTGTGTTGACACAGTCTCCAGCCACCCTGTCTTTGTCTCCAGGGGAAAGAG
CCACCCTCTCCTGCAGGGCCAGTCAGAGTGTTAGCAGCTACTTAGCCTGGTACCA
ACAGAAACCTGGCCAGGCTCCCAGGCTCCTCATCTATGATGCATCCAACAGGGCC
ACTGGCATCCCAGCCAGGTTCAGTGGCAGTGGGTCTGGGACAGACTTCACTCTCA
CCATCAGCAGCCTAGAGCCTGAAGATTTTGCAGTTTATTACTGTCAGCAGCGTAG
CAACTGGCCTCCGCTCACTTTCGGCGGAGGGACCAAGGTGGAGATCAAA
Heavy Chain → 491-2B5 (LJI_0491-113)

| | | |
|---|---|---|
| CRDH1 | SEQ ID NO: 131 | GGSFSGYY |
| CRDH2 | SEQ ID NO: 132 | INHSGST |
| CRDH3 | SEQ ID NO: 133 | ARVVIPFFYFDY |

AA: SEQ ID NO: 134
QVQLQQWGAGLLKPSETLSLTCAVYGGSFSGYYWSWIRQPPGKGLEWIGEINHSGS
TNYNPSLKSRVTISVDTSKNHFSLKLSSVTAADTAVYYCARVVIPFFYFDYWGQGTL
VTVS
Nt: SEQ ID NO: 135
CAGGTGCAGCTACAACAGTGGGGCGCAGGACTGTTGAAGCCTTCGGAGACCCTG
TCCCTCACCTGCGCTGTCTATGGTGGGTCCTTCAGTGGTTACTACTGGAGCTGGAT
CCGCCAGCCCCCAGGGAAGGGGCTGGAGTGGATTGGGGAAATCAATCATAGTGG
AAGCACCAACTACAACCCGTCCCTCAAGAGTCGAGTCACCATATCAGTGGACACG
TCCAAGAACCACTTCTCCCTGAAGCTGAGCTCTGTGACCGCCGCGGACACGGCTG
TGTATTACTGTGCGAGAGTGGTAATTCCTTTTTTCTACTTTGACTACTGGGGCCAG
GGAACCCTGGTCACCGTCTCC
Light Chain → 491-2B5 (LJI_0491-113)

| | | |
|---|---|---|
| CDRL1 | SEQ ID NO: 136 | NIGSK |
| CDRL2 | SEQ ID NO: 137 | YDS |
| CDRL3 | SEQ ID NO: 138 | QVWDSSDPLYV |

AA: SEQ ID NO: 139
QAGLTQPPSVSVAPGKTARITCGGNNIGSKSVHWYQQKPGQAPVLVIYYDSDRPSGI
PERFSGSNSGNTATLTISRVEAGDEADYYCQVWDSSDPLYVFGTGTKLTVL
Nt: SEQ ID NO: 140
CAGGCAGGGCTGACTCAGCCACCCTCAGTGTCAGTGGCCCCAGGAAAGACGGCC
AGGATTACCTGTGGGGGAAACAACATTGGAAGTAAAAGTGTGCACTGGTACCAG
CAGAAGCCAGGCCAGGCCCCTGTGCTGGTCATCTATTATGATAGCGACCGGCCCT
CAGGGATCCCTGAGCGATTCTCTGGCTCCAACTCTGGGAACACGGCCACCCTGAC
CATCAGCAGGGTCGAAGCCGGGGATGAGGCCGACTATTACTGTCAGGTGTGGGA
TAGTAGTGATCCTCTTTATGTCTTCGGAACTGGGACCAAGCTTACCGTCCTA
Heavy Chain → 491-2B7 (LJI_0491-115).1

| | | |
|---|---|---|
| CRDH1 | SEQ ID NO: 141 | GFIFSSYA |
| CRDH2 | SEQ ID NO: 142 | ISGSGGST |
| CRDH3 | SEQ ID NO: 143 | AKQCRTDGVCYTALFDY |

AA: SEQ ID NO: 144
EVQLVQSGGGLVQPGGSLRLSCAASGFIFSSYAMSWVRQAPGKGLEWVSAISGSGG
STYYADSVKGRFTISRDNSKNTLYLQMNSLRAEDTAVYYCAKQCRTDGVCYTALF
DYWGQGSLVTVS
Nt: SEQ ID NO: 145
GAGGTGCAGCTGGTGCAGTCTGGGGGAGGCTTGGTACAGCCTGGGGGGTCCCTG
AGACTCTCCTGTGCAGCCTCTGGATTCATCTTTAGCAGCTATGCCATGAGCTGGGT
CCGCCAGGCTCCAGGGAAGGGGCTGGAGTGGGTCTCAGCTATTAGTGGTAGTGGT
GGTAGCACATACTACGCAGACTCCGTGAAGGGCCGATTCACCATCTCCAGAGACA
ATTCCAAGAACACGCTGTATCTGCAAATGAACAGCCTGAGAGCCGAGGACACGG
CCGTTTATTACTGTGCGAAACAATGTAGAACTGATGGTGTATGCTATACGGCTTT
ATTTGACTACTGGGGCCAGGGAAGCCTGGTCACCGTCTCC
Light Chain → 491-2B7 (LJI_0491-115)

| | | |
|---|---|---|
| CDRL1 | SEQ ID NO: 151 | SSNIGSNY |
| CDRL2 | SEQ ID NO: 152 | GNN |
| CDRL3 | SEQ ID NO: 153 | AAWDDSLSGRWV |

AA: SEQ ID NO: 154
QPVLTQPPSASGTPGQRVTISCSGSSSNIGSNYVYWYQQLPGTAPKLLIDGNNQRPSG
VPDRFSGSKSGTSASLAISGLRSEDEADYYCAAWDDSLSGRWVFGGGTKLTVL
Nt: SEQ ID NO: 155
CAGCCTGTGCTGACTCAGCCACCCTCAGCGTCTGGGACCCCCGGGCAGAGGGTCA
CCATCTCTTGTTCCGGAAGCAGTTCCAACATCGGAAGTAATTATGTATACTGGTA
CCAGCAGCTCCCAGGAACGGCCCCCAAACTCCTCATCGATGGGAATAATCAGCG
GCCCTCAGGGGTCCCTGACCGATTCTCTGGCTCCAAGTCTGGCACCTCAGCCTCCC
TGGCCATCAGTGGGCTCCGGTCCGAGGATGAGGCTGATTATTACTGTGCAGCATG
GGATGACAGCCTGAGTGGCCGTTGGGTGTTCGGCGGAGGGACCAAGCTTACCGTC
CTA
Heavy Chain → 491-2B7 (LJI_0491-115).2

| | | |
|---|---|---|
| CRDH1 | SEQ ID NO: 146 | GFIFSSYA |
| CRDH2 | SEQ ID NO: 147 | ISGSGGST |
| CRDH3 | SEQ ID NO: 148 | AKQCRTDGVCYTALFDY |

AA: SEQ ID NO: 149
EVQLLESGGGLVQPGGSLRLSCAASGFIFSSYAMSWVRQAPGKGLEWVSAISGSGGS
TYYADSVKGRFTISRDNSKNTLYLQMNSLRAEDTAVYYCAKQCRTDGVCYTALFD
YWGQGSLVTVS
Nt: SEQ ID NO: 150
GAGGTGCAGCTGTTGGAGTCTGGGGGAGGCTTGGTACAGCCTGGGGGGTCCCTG
AGACTCTCCTGTGCAGCCTCTGGATTCATCTTTAGCAGCTATGCCATGAGCTGGGT
CCGCCAGGCTCCAGGGAAGGGGCTGGAGTGGGTCTCAGCTATTAGTGGTAGTGGT
GGTAGCACATACTACGCAGACTCCGTGAAGGGCCGATTCACCATCTCCAGAGACA
ATTCCAAGAACACGCTGTATCTGCAAATGAACAGCCTGAGAGCCGAGGACACGG
CCGTTTATTACTGTGCGAAACAATGTAGAACTGATGGTGTATGCTATACGGCTTT
ATTTGACTACTGGGGCCAGGGAAGCCTGGTCACCGTCTCC
Light Chain → 491-2B7 (LJI_0491-115)

| | | |
|---|---|---|
| CDRL1 | SEQ ID NO: 151 | SSNIGSNY |
| CDRL2 | SEQ ID NO: 152 | GNN |
| CDRL3 | SEQ ID NO: 153 | AAWDDSLSGRWV |

AA: SEQ ID NO: 154
QPVLTQPPSASGTPGQRVTISCSGSSSNIGSNYVYWYQQLPGTAPKLLIDGNNQRPSG
VPDRFSGSKSGTSASLAISGLRSEDEADYYCAAWDDSLSGRWVFGGGTKLTVL
Nt: SEQ ID NO: 155
CAGCCTGTGCTGACTCAGCCACCCTCAGCGTCTGGGACCCCCGGGCAGAGGGTCA
CCATCTCTTGTTCCGGAAGCAGTTCCAACATCGGAAGTAATTATGTATACTGGTA
CCAGCAGCTCCCAGGAACGGCCCCCAAACTCCTCATCGATGGGAATAATCAGCG
GCCCTCAGGGGTCCCTGACCGATTCTCTGGCTCCAAGTCTGGCACCTCAGCCTCCC
TGGCCATCAGTGGGCTCCGGTCCGAGGATGAGGCTGATTATTACTGTGCAGCATG
GGATGACAGCCTGAGTGGCCGTTGGGTGTTCGGCGGAGGGACCAAGCTTACCGTC
CTA
Heavy Chain → 491-2B8 (LJI_0491-116).1

| | | |
|---|---|---|
| CRDH1 | SEQ ID NO: 156 | GYTFTTFG |
| CRDH2 | SEQ ID NO: 157 | ISTYNGNT |
| CRDH3 | SEQ ID NO: 158 | TRGGSRYNWNDFDYYNYYMDV |

AA: SEQ ID NO: 159
QVQLVQSGAEVKKPGASVKVSCKASGYTFTTFGISWVRQAPGQGLEWMGWISTYN
GNTKYAQKLQGRVTMTTDTSTSTAYMELRSLRSDDTAVYFCTRGGSRYNWNDFD
YYNYYMDVWGKGTTVTVS
Nt: SEQ ID NO: 160
CAGGTTCAGCTGGTGCAGTCTGGAGCTGAGGTGAAGAAGCCTGGGGCCTCAGTG
AAGGTCTCCTGCAAGGCTTCAGGTTACACCTTTACCACCTTTGGTATCAGCTGGGT
GCGACAGGCCCCTGGACAAGGGCTTGAGTGGATGGGATGGATCAGCACTTACAA
TGGTAACACAAAGTATGCACAGAAGCTCCAGGGCAGAGTCACCATGACCACAGA
CACATCCACGAGCACAGCCTACATGGAGCTGAGGAGCCTGAGATCTGACGACAC
GGCCGTGTATTTCTGTACGAGAGGGGGCTCTAGGTATAACTGGAACGACTTCGAC
TACTACAACTACTACATGGACGTCTGGGGCAAAGGGACCACGGTCACCGTCTCC
Light Chain → 491-2B8 (LJI_0491-116).1

| | | |
|---|---|---|
| CDRL1 | SEQ ID NO: 161 | QSLVYSDGNTY |
| CDRL2 | SEQ ID NO: 162 | KVS |
| CDRL3 | SEQ ID NO: 163 | MQGTHWPF |

AA: SEQ ID NO: 164
EIVMTQSPLSLPVTLGQPASISCRSSQSLVYSDGNTYLNWFQQRPGQSPRRLIYKVSN
RDSGVPDRFSGSGSGTNFTLKISRVEAEDVGVYYCMQGTHWPFFGGGTKVEIK
Nt: SEQ ID NO: 165
GAAATTGTAATGACACAGTCTCCACTCTCCCTGCCCGTCACCCTTGGACAGCCGG
CCTCCATCTCCTGCAGGTCTAGTCAAAGCCTCGTATACAGTGATGGAAACACCTA
CTTGAATTGGTTTCAGCAGAGGCCAGGCCAATCTCCAAGGCGCCTAATTTATAAG
GTTTCTAACCGGGACTCTGGGGTCCCAGACAGATTCAGCGGCAGTGGGTCAGGCA
CTAATTTCACACTGAAAATCAGCAGGGTGGAGGCTGAGGATGTTGGGGTTTATTA
CTGCATGCAAGGTACACACTGGCCTTTTTTCGGCGGAGGGACCAAGGTGGAAATC
AAA
Heavy Chain → 491-2B8 (LJI_0491-116).2

| | | |
|---|---|---|
| CRDH1 | SEQ ID NO: 166 | GYTFTTFG |
| CRDH2 | SEQ ID NO: 167 | ISTYNGNT |
| CRDH3 | SEQ ID NO: 168 | TRGGSRYNWYDFDYYNYYMDV |

AA: SEQ ID NO: 169
QVQLVQSGAEVKKPGASVKVSCKASGYTFTTFGISWVRQAPGQGLEWMGWISTYN
GNTKYAQKLQGRVTMTTDTSTSTAYMELRSLRSDDTAVYFCTRGGSRYNWYDFD
YYNYYMDVWGKGTTVTVS
Nt: SEQ ID NO: 170
CAGGTGCAGCTGGTGCAGTCTGGAGCTGAGGTGAAGAAGCCTGGGGCCTCAGTG
AAGGTCTCCTGCAAGGCTTCAGGTTACACCTTTACCACCTTTGGTATCAGCTGGGT
GCGACAGGCCCCTGGACAAGGGCTTGAGTGGATGGGATGGATCAGCACTTACAA
TGGTAACACAAAGTATGCACAGAAGCTCCAGGGCAGAGTCACCATGACCACAGA
CACATCCACGAGCACAGCCTACATGGAGCTGAGGAGCCTGAGATCTGACGACAC
GGCCGTGTATTTCTGTACGAGAGGGGGCTCTAGGTATAACTGGTACGACTTCGAC
TACTACAACTACTACATGGACGTCTGGGGCAAAGGGACCACGGTCACCGTCTCC
Light Chain → 491-2B8 (LJI_0491-116).2

| | | |
|---|---|---|
| CDRL1 | SEQ ID NO: 171 | QSLVYSDGNTY |
| CDRL2 | SEQ ID NO: 172 | KVS |
| CDRL3 | SEQ ID NO: 173 | MQGTHWPF |

AA: SEQ ID NO: 174
DVVMTQSPLSLPVTLGQPASISCRSSQSLVYSDGNTYLNWFQQRPGQSPRRLIYKVS
NRDSGVPDRFSGSGSGTNFTLKISRVEAEDVGVYYCMQGTHWPFFGGGTKVEIK
Nt: SEQ ID NO: 175
GATGTTGTGATGACTCAGTCTCCACTCTCCCTGCCCGTCACCCTTGGACAGCCGGC
CTCCATCTCCTGCAGGTCTAGTCAAAGCCTCGTATACAGTGATGGAAACACCTAC
TTGAATTGGTTTCAGCAGAGGCCAGGCCAATCTCCAAGGCGCCTAATTTATAAGG
TTTCTAACCGGGACTCTGGGGTCCCAGACAGATTCAGCGGCAGTGGGTCAGGCAC
TAATTTCACACTGAAAATCAGCAGGGTGGAGGCTGAGGATGTTGGGGTTTATTAC
TGCATGCAAGGTACACACTGGCCTTTTTTCGGCGGAGGGACCAAGGTGGAGATCA
AA
Heavy Chain → 491-2G3 (LJI_0491-171)

| | | |
|---|---|---|
| CRDH1 | SEQ ID NO: 176 | FTFDEYA |
| CRDH2 | SEQ ID NO: 177 | ISWNSGRL |
| CRDH3 | SEQ ID NO: 178 | AKSSRFLEWLLPGDAFDI |

AA: SEQ ID NO: 179
QVQLVESGGGWVQPGRSRRLSCAASGFTFDEYAMHWVRQTPGKGLEWVSGISWNS
GRLDYADSVKGRFTISRDNAKNSLYLQMNSLRAEDTALYYCAKSSRFLEWLLPGD
AFDIWGRGTMVSVS
Nt: SEQ ID NO: 180
CAGGTACAGCTCGTGGAGTCTGGGGGAGGCTGGGTACAGCCTGGCAGGTCCCGG
AGACTCTCCTGTGCAGCCTCTGGATTCACCTTTGATGAGTATGCCATGCACTGGGT
CCGACAAACTCCAGGGAAGGGCCTGGAGTGGGTCTCAGGTATTAGTTGGAATAG
TGGTCGCCTAGACTATGCGGACTCTGTGAAGGGCCGATTCACCATCTCCAGAGAC
AACGCCAAGAACTCCCTGTATCTGCAAATGAACAGTCTGAGAGCTGAGGACACG
GCCTTGTATTACTGTGCAAAATCTTCACGATTTTTGGAGTGGTTATTGCCCGGTGA
TGCTTTTGATATCTGGGGCCGAGGGACAATGGTCTCCGTCTCT
Light Chain → 491-2G3 (LJI_0491-171).1

| | | |
|---|---|---|
| CDRL1 | SEQ ID NO: 181 | QSVSDY |
| CDRL2 | SEQ ID NO: 182 | DAS |
| CDRL3 | SEQ ID NO: 183 | QQRSNWPPIT |

AA: SEQ ID NO: 184
DIVLTQSPATLSLSPGERATLSCRSSQSVSDYLAWYQQKPGQAPRLLIYDASNRATGIP
ARFSGSGSGTDFTLTISSLEPEDFAVYYCQQRSNWPPITFGQGTRLEIK
Nt: SEQ ID NO: 185
GACATTGTGCTGACCCAGTCTCCAGCCACCCTGTCTTTGTCTCCAGGGGAAAGAG
CCACCCTCTCCTGCAGGTCCAGTCAGAGTGTTAGCGACTACTTAGCCTGGTACCA
ACAGAAACCTGGCCAGGCTCCCAGGCTCCTCATCTATGATGCATCCAACAGGGCC
ACTGGCATCCCAGCCAGGTTCAGTGGCAGTGGGTCTGGGACAGACTTCACTCTCA
CCATCAGCAGCCTAGAGCCTGAAGATTTTGCAGTTTATTACTGTCAACAGCGTAG
CAACTGGCCTCCGATCACCTTCGGCCAAGGGACACGACTGGAGATTAAA
Heavy Chain → 491-2G3 (LJI_0491-171)

| | | |
|---|---|---|
| CRDH1 | SEQ ID NO: 176 | FTFDEYA |
| CRDH2 | SEQ ID NO: 177 | ISWNSGRL |
| CRDH3 | SEQ ID NO: 178 | AKSSRFLEWLLPGDAFDI |

AA: SEQ ID NO: 179
QVQLVESGGGWVQPGRSRRLSCAASGFTFDEYAMHWVRQTPGKGLEWVSGISWNS
GRLDYADSVKGRFTISRDNAKNSLYLQMNSLRAEDTALYYCAKSSRFLEWLLPGD
AFDIWGRGTMVSVS
Nt: SEQ ID NO: 180
CAGGTACAGCTCGTGGAGTCTGGGGGAGGCTGGGTACAGCCTGGCAGGTCCCGG
AGACTCTCCTGTGCAGCCTCTGGATTCACCTTTGATGAGTATGCCATGCACTGGGT
CCGACAAACTCCAGGGAAGGGCCTGGAGTGGGTCTCAGGTATTAGTTGGAATAG
TGGTCGCCTAGACTATGCGGACTCTGTGAAGGGCCGATTCACCATCTCCAGAGAC
AACGCCAAGAACTCCCTGTATCTGCAAATGAACAGTCTGAGAGCTGAGGACACG
GCCTTGTATTACTGTGCAAAATCTTCACGATTTTTGGAGTGGTTATTGCCCGGTGA
TGCTTTTGATATCTGGGGCCGAGGGACAATGGTCTCCGTCTCT
Light Chain → 491-2G3 (LJI_0491-171).2

| | | |
|---|---|---|
| CDRL1 | SEQ ID NO: 186 | QSVSDY |
| CDRL2 | SEQ ID NO: 187 | DAS |
| CDRL3 | SEQ ID NO: 188 | QQRSNWPPIT |

AA: SEQ ID NO: 189
EIVLTQSPATLSLSPGERATLSCRSSQSVSDYLAWYQQKPGQAPRLLIYDASNRATGIP
ARFSGSGSGTDFTLTISSLEPEDFAVYYCQQRSNWPPITFGQGTRLEIK
Nt: SEQ ID NO: 190
GAAATTGTGTTGACACAGTCTCCAGCCACCCTGTCTTTGTCTCCAGGGGAAAGAG
CCACCCTCTCCTGCAGGTCCAGTCAGAGTGTTAGCGACTACTTAGCCTGGTACCA
ACAGAAACCTGGCCAGGCTCCCAGGCTCCTCATCTATGATGCATCCAACAGGGCC
ACTGGCATCCCAGCCAGGTTCAGTGGCAGTGGGTCTGGGACAGACTTCACTCTCA
CCATCAGCAGCCTAGAGCCTGAAGATTTTGCAGTTTATTACTGTCAACAGCGTAG
TAACTGGCCTCCGATCACCTTCGGCCAAGGGACACGACTGGAGATTAAA
Heavy Chain → 491-4H4 (LJI_0491-278)

| | | |
|---|---|---|
| CRDH1 | SEQ ID NO: 191 | GFTVSSNY |
| CRDH2 | SEQ ID NO: 192 | YSGGST |
| CRDH3 | SEQ ID NO: 193 | ARAPYGDSH |

AA: SEQ ID NO: 194
QVQLVESGGGLIQPGGSLRLSCVASGFTVSSNYMSWFRQGPGKGLEWVSVIYSGGS
TFHADSVKGRFTISRDHSKNTLYLQMNSLRAEDTAVYYCARAPYGDSHWGQGTLV
TVSS
Nt: SEQ ID NO: 195
CAGGTACAGCTCGTGGAGTCTGGAGGAGGCTTGATCCAGCCTGGGGGGTCCCTGA
GACTCTCCTGTGTAGCCTCTGGGTTCACCGTCAGTAGCAACTACATGAGCTGGTTC
CGCCAGGGTCCAGGGAAGGGGCTGGAGTGGGTCTCAGTTATTTATAGCGGTGGTA
GCACATTCCATGCAGACTCCGTGAAGGGCCGATTCACCATCTCCAGAGACCATTC
CAAGAACACGCTGTATCTTCAAATGAACAGCCTGAGAGCCGAGGACACGGCCGT
GTATTACTGTGCGAGAGCTCCCTACGGTGACTCCCACTGGGGCCAGGGAACCCTG
GTCACCGTCTCCTCAG
Light Chain → 491-4H4 (LJI_0491-278)

| | | |
|---|---|---|
| CDRL1 | SEQ ID NO: 196 | QDIRNF |
| CDRL2 | SEQ ID NO: 197 | DAS |
| CDRL3 | SEQ ID NO: 198 | QQYDNLPIT |

AA: SEQ ID NO: 199
DIVMTQSPSSLSASIGDRVTITCQASQDIRNFLNWYQQKPGKAPKLLIHDASNLETGV
PSRFSGSGSGTDFTFTISSLQPEDIATYYCQQYDNLPITFGQGTRLEIK
Nt: SEQ ID NO: 200
GATATTGTGATGACTCAGTCTCCATCCTCCCTGTCTGCATCTATAGGAGACAGAGT
CACCATCACTTGCCAGGCGAGTCAGGACATTCGGAACTTTTTAAATTGGTATCAG
CAGAAACCAGGGAAAGCCCCTAAGCTCCTGATCCACGATGCATCCAATTTGGAA
ACAGGGGTCCCATCAAGGTTCAGTGGAAGTGGATCTGGGACAGATTTTACTTTCA
CCATCAGCAGCCTGCAGCCTGAAGATATTGCAACATATTACTGTCAACAGTATGA
TAATCTCCCCATCACCTTCGGCCAAGGGACACGACTGGAGATTAAA

Example 2. The present invention includes two unique human antibodies produced by memory B-cells that produce antibodies targeting the Delta and Omicron variants of the Spike protein of SARS-CoV-2 (SARS2-S).

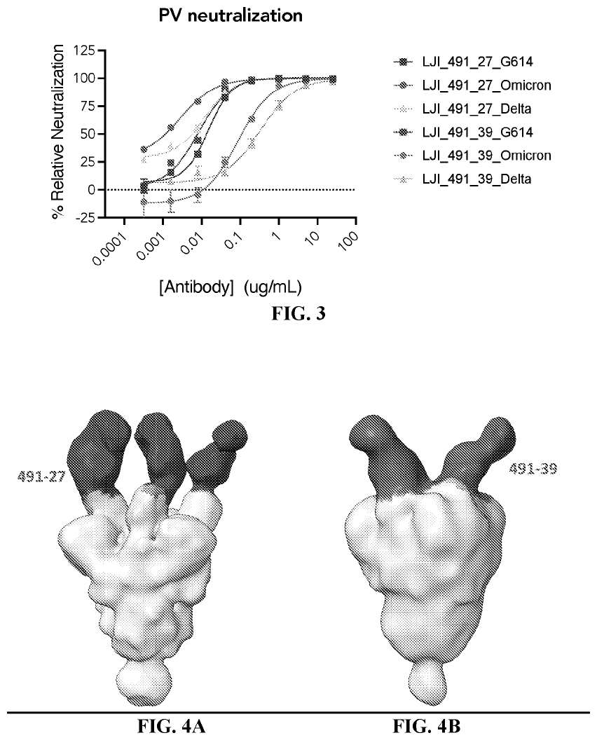

Neutralization of SARS-CoV2 pseudovirions. Pre-titrated amounts of rVSV-SARS-CoV-2 was incubated with serially diluted purified monoclonal antibody at 37° C. for 1 hr before addition to confluent Vero monolayers in 96-well plates. Infection proceeded for 12-16 hrs at 37° C. in 5% CO₂ before cells were fixed in 4% paraformaldehyde and stained with 10 ug/mL Hoescht. Cells were imaged using a CellInsight CX5 imager and infection was quantitated by automated enumeration of total cells and those expressing GFP. Infection was normalized to the average number of cells infected with rVSV-SARS-CoV-2 incubated with virus only. Data are presented as the relative neutralization for each concentration of antibody. Neutralization IC50s (displayed as ng/mL) were calculated using non-linear regression in GraphPad Prism 9.0. The antibodies also bind to D614G mutated Spike. FIG. 3 is a graph that shows the relative neutralization for each concentration of antibody in Vero monolayers infected with rVSV-SARS-CoV-2.

TABLE 1
Antibody Neutralization of SARS-CoV2 pseudovirions.

| | rVSV-G614 | | rVSV-Delta | | rVSV-Omicron | |
|---|---|---|---|---|---|---|
| | IC50 | IC90 | IC50 | IC90 | IC50 | IC90 |
| | (ng/mL) | (ng/mL) | (ng/mL) | (ng/mL) | (ng/mL) | (ng/mL) |
| LJI_491-27 (1C3) | 8 | 69 | 12 | 79 | 3 | 28 |
| LJI_491-39 (2E6) | 15 | 66 | 324 | 3236 | 88 | 784 |

Negative stain electron microscopy. Purified IgGs of each antibody was complexed with the ectodomain of SARS-CoV2 Spike. (FIG. 4A) Three antigen-binding fragments (Fab) of mAb 491-27 (1C3) (top left) bind to “up” oriented receptor-binding domains (RBD) of Spike. (FIG. 4B) Two Fabs of mAb 491-39 (2E6) (top right) are bound to “down” oriented RBDs of Spike.

### Example 2. Novel Antibodies

Complementarity determining regions (CDR)1, CDR2, and CDR3 sequences, respectively:

```
HC CDR 27
SEQ ID NO: 201
GFTFDDYA
SEQ ID NO: 202
SSWNSGSV
SEQ ID NO: 203
AKAVDPTRGSYSPDYGFDI
HC CDR 39
SEQ ID NO: 204
GFTFSGSA
SEQ ID NO: 205
IRSKANSYAT
SEQ ID NO: 206
TREDIVGVPVAKD
LC CDR 29
SEQ ID NO: 207
QSISSY
SEQ ID NO: 208
AAS
SEQ ID NO: 209
QQSYSTPLT
LC CDR 39
SEQ ID NO: 207
QSISSY
SEQ ID NO: 208
AAS
SEQ ID NO: 210
QQSYSTLSIT
Antibody sequences.
>LJI_491_27_HC (1C3)
SEQ ID NO: 211
                                                    CDR H1
QVQLLESGGGLVQPGRSLRLSCAASGFTFDDYAMHWVRQPPGKGLEWV
      CDR H2
SGSSWNSGSVVYADSVKGRFTISRDSAKNSLHLQMNSLRVEDTALYYC
          CDR H3
AKAVDPTRGSYSPDYGFDIWGQGTMVTVS
LJI_491_27_HC (1C3)
SEQ ID NO: 212
GAAGTGCAGCTGGTGGAGTTTGGGGGAGGCTTGGTACAGCCTGGCAGG
TCCCTGAGACTTTCCTGTGCAGCCTTTGGATTCACCTTTGATGATTAT
GCCATGCACTGGGTCCGGCAACCTCCAGGGAAGGGCCTGGAGTGGGTT
TCAGGTAGTAGTTGGAATAGTGGTAGCGTAGTCTATGCGGACTCTGTG
AAGGGCCGATTCACCATCTCCAGAGACAGCGCCAAGAACTCCCTGCAT
TTGCAAATGAACAGTTTGAGAGTTGAGGACACGGCCTTATATTACTGT
GCAAAAGCCGTCGACCCTACACGTGGGAGCTACTCCCCAGATTATGGT
TTTGATATCTGGGGCCAAGGGACAATGGTCACCGTCTCT
>LJI_491_27_kLC (1C3)
SEQ ID NO: 213
                                                    CDR L1
DIQMTQSPSSLSASVGDRVTITCRASQSISSYLNWYQQKPGKAPNLLI
CDR L2 CDR L3
YAASSLESGVPSRFSGSGSGTDFTLTISSLQPEDFATYYCQQSYSTPL
TFGGGTKVEIKRTV
LJI_491_27_kLC (1C3)
SEQ ID NO: 214
GACATCCAGATGACCCAGTCTCCATCCTCCCTGTcTGCATCTGTAGGA
GACAGAGTCACCATCACTTGCCGGGCAAGTCAGAGCATCAGCAGCTAT
TTAAATTGGTATCAGCAGAAACCAGGGAAAGCCCCTAACCTCCTGATC
TATGCTGCATCCAGTTTGGAAAGTGGGGTCCCATCAAGGTTCAGTGGC
AGTGGATCTGGGACAGATTTCACTCTCACCATCAGCAGCCTGCAACCT
GAAGATTTTGCAACTTACTACTGTCAACAGAGTTACAGTACCCCGCTC
ACTTTCGGCGGAGGGACCAAGGTG
>LJI_491_39_HC (2E6)
SEQ ID NO: 215
                                                    CRD H1
EVQLVQSGGGLVQPGGSLKLSCAASGFTFSGSAMHWVRQASGKGLEWV
        CDR H2
GHIRSKANSYATAYAASKGRFTISRDDSKNTAYLQMNSLKTEDTAVYY
          CDR H3
CTREDIVGVPVAKDWFDPWGQGTLVTVS
LJI_491_39_HC (2E6)
SEQ ID NO: 216
GAAGTGCAGCTGGTGCAGTCTGGGGGAGGCTTGGTCCAGCCTGGGGGG
TCCCTGAAACTCTCCTGTGCAGCCTCTGGGTTCACCTTCAGTGGCTCT
GCTATGCACTGGGTCCGCCAGGCTTCCGGGAAAGGGCTGGAGTGGGTT
GGCCATATTAGAAGCAAAGCTAACAGTTACGCGACAGCATATGCTGCG
TCGGTGAAAGGCAGGTTCACCATCTCCAGAGATGATTCAAAGAACACG
GCGTATCTGCAAATGAACAGCCTGAATACCGAGGACACGGCCGTGTAT
TACTGTACTAGAGAGGATATTGTAGGAGTACCAGTTGCTAAGGACTGG
TTCGACCCCTGGGGCCAGGGAACCCTGGTCACCGTC
>LJI_491_39_kLC (2E6)
SEQ ID NO: 217
                                                    CDR L1
DIQMTQSPSSLFASVGDRVTITCRASQSISSYLNWYQQKPGKAPKLLI
CDR L2 CDR L3
YAASSLQSGVPSRFSGSGFGTDFTLTISSLQPEDFATYYCQQSYSTLS
ITFGQGTRLEIKRTV
LJI_491_39_kLC (2E6)
SEQ ID NO: 218
GACATCCAGATGACGCAGTCTCCATCCTCCCTGTTTGCATCTGTAGGA
GACAGAGTCACCATCACTTGCCGGGCAAGTCAGAGCATTAGCAGCTAT
TTAAATTGGTATCAGCAGAAACCAGGGAAAGCCCCTAAGCTCCTGATC
TATGCTGCATCCAGTTTGCAAAGTGGGGTCCCATCAAGGTTCAGTGGC
AGTGGATTTGGGACAGATTTCACTCTCACCATCAGCAGTCTGCAACCT
GAAGATTTTGCAACTTACTACTGTCAACAGAGTTACAGTACCCTTTCG
ATCACCTTCGGCCAAGGGACACGACTGGAGATTAAACGAACTGTG
```

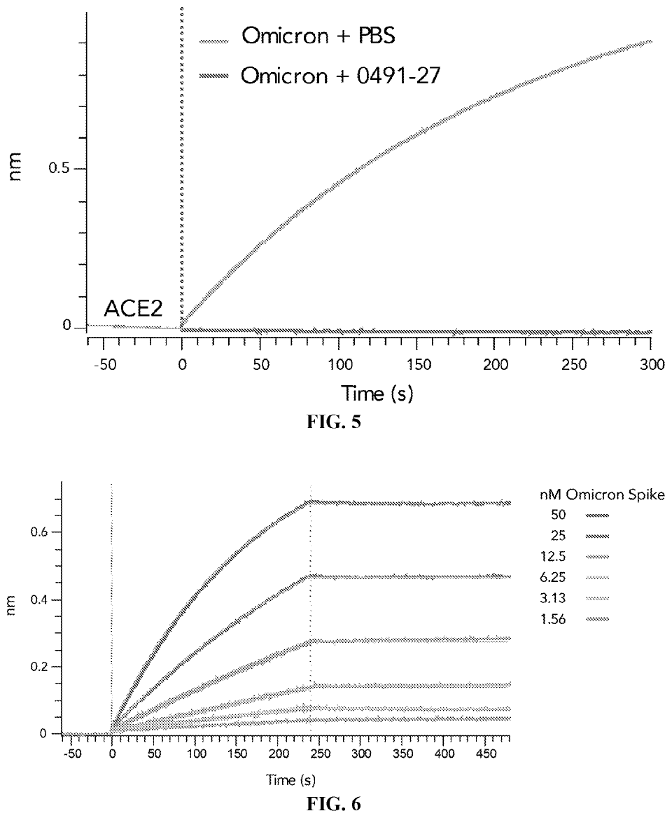

FIG. 5 is a graph that shows 100% ACE2 blocking with the 0491-27 (1C3) antibody of the present invention. FIG. 6 is a graph that shows the high-affinity on-rate of the 0491-27 (1C3) antibody of the present invention.

Comparative Neutralization of rVSV-based pseudoparticles.

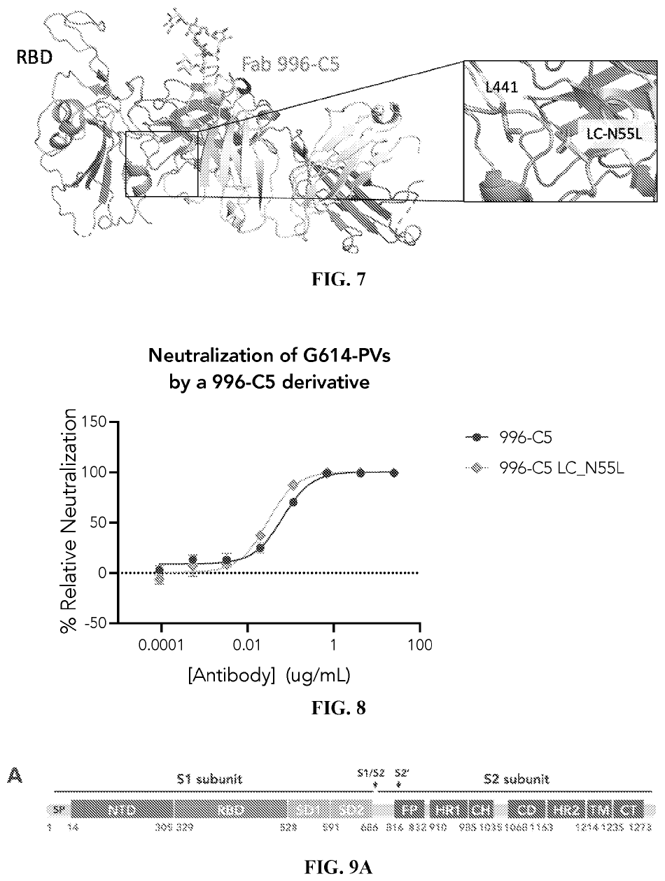

Structure of 996-C5 Fab bound to SARS-CoV-2 RBD. FIG. 7 shows the receptor binding domain (RBD) of SARS-CoV-2 in complex with the antigen-binding fragment (Fab) of antibody 996-C5, determined to 2 Å resolution. Mutation of light chain residue N55 to leucine enhances the hydrophobic packing interaction with RBD residue L441 (gray).

Pre-titrated amounts of rVSV-SARS-CoV-2 were incubated with serially diluted purified monoclonal antibody at 37° C. for 1 hour before addition to confluent Vero monolayers in 96-well plates. Infection proceeded for 12-16 hours at 37° C. in 5% C02 before cells were fixed in 4% paraformaldehyde and stained with 10 ug/mL Hoescht. Cells were imaged using a CellInsight CX5 imager and infection was quantitated by automated enumeration of total cells and those expressing GFP. Infection was normalized to the average number of cells infected with rVSV-SARS-CoV-2 incubated with virus only. Data are presented as the relative neutralization for each concentration of antibody. Neutralization IC50s (displayed as ng/mL) were calculated using non-linear regression in GraphPad Prism 9.0. An exemplary neutralization curve is shown in FIG. 8.

TABLE 1
Comparative neutralization for each concentration of antibody.

| | 996-C5 | | 996-C5 LC_N55L | |
|---|---|---|---|---|
| | IC50 (ng/mL) | IC90 (ng/mL) | IC50 (ng/mL) | IC90 (ng/mL) |
| G614 | 62 | 493 | 36 | 299 |
| Beta | 45 | 414 | 25 | 187 |
| Delta | 62 | 700 | 30 | 313 |
| Lambda | 74 | 408 | 35 | 297 |
| Omicron | 169 | 1074 | 110 | 552 |

### Antibody Sequences:

```
Heavy Chain Complementarity Determining Regions
(CDRH1 to CDRH3)
CDRH1
SEQ ID NO: 219
GFTFSYYW
CDRH2
SEQ ID NO: 220
IKQDGSEKY
CDRH3
SEQ ID NO: 221
ARDRYGWPSIPAPFDY
Light Chain Complementarity Determining Regions
(CDRL1 to CDRL3)
CDRL1
SEQ ID NO: 222
SSDIGGYDY
CDRL2
SEQ ID NO: 223
DVSL
CDRL3
SEQ ID NO: 224
NSYTNISTLGV
The bolded leucine in CDRL2 is the variant of
the present invention.
Heavy Chain (996-C5)
>996-C5_HC
SEQ ID NO: 225
                                                    CRD H1
QVQLVESGGGLVQPGGSLRLSCAASGFTFSYYWMSWVRQAPGKGLEWV
        CDR H2
ANIKQDGSEKYYVDSVKGRFTISRDNAKNSLYLQMNSLRAEDTAVYYC
          CDR H3
ARDRYGWPSIPAPFDYWGQGTLVTVS
Heavy Chain
SEQ ID NO: 226
CAAGTTCAATTGGTGGAGAGCGGGGGAGGGTTGGTTCAACCAGGCGGC
AGTCTGCGCTTGTCCTGTGCGGCATCAGGGTTTACCTTCAGCTACTAC
TGGATGAGTTGGGTGCGGCAGGCTCCAGGAAAAGGACTTGAATGGGTA
GCAAATATTAAACAGGATGGGAGCGAAAAATACTACGTCGACTCCGTC
AAAGGAAGATTCACTATAAGCCGGGATAACGCGAAAAACTCTTTGTAC
CTTCAAATGAACTCTCTCAGAGCTGAGGATACCGCCGTCTATTATTGT
GCCAGGGATCGGTATGGGTGGCCAAGTATCCCAGCGCCCTTTGATTAC
TGGGGCCAAGGGACTCTTGTTACCGTGTCTAGC
Light Chain
>996-C5_LLC (N55L mutation in bold italics) of
the present invention.
SEQ ID NO: 227
                                                      CRD L1
QSALTQPASVSGSPGQSITISCTGTSSDIGGYDYVSWYQHHPGKAPKL
    CDR L2 CDR L3
MIYDVSLRPSGVSNRFSGSKSGNTASLTISGLQAEDEADYYCNSYTNI
STLGVFGGGTKLTVL
```

The bolded, underlined and italicized Leucine in CDRL2 is the variant of the present invention (N55L mutation).

```
Light Chain
>996-C5 (Parent antibody).
SEQ ID NO: 228
                                                    CRD L1
QSALTQPASVSGSPGQSITISCTGTSSDIGGYDYVSWYQHHPGKAPKL
    CDR L2 CDR L3
MIYDVSNRPSGVSNRFSGSKSGNTASLTISGLQAEDEADYYCNSYTNI
STLGVFGGGTKLTVL
The bolded, underlined and italicized Asparagine
in CDRL2 is the original sequence.
>996-C5_LLC (N55L mutation indicated in bold)
(SEQ ID NO: 229)
CAATCAGCGCTTACCCAGCCTGCAAGTGTTAGCGGGAGCCCAGGGCAG
TCTATAACAATTTCCTGTACTGGTACAAGCTCCGACATCGGGGGGTAC
GATTATGTCAGTTGGTACCAACACCATCCCGGCAAAGCTCCAAAACTT
ATGATTTACGACGTAAGCCTTCGACCCAGCGGGGTGAGCAACAGGTTC
AGTGGGTCAAAGAGCGGGAACACAGCCTCACTTACAATAAGTGGACTT
CAGGCTGAGGATGAGGCTGACTATTACTGTAATAGTTATACTAACATT
TCAACGCTTGGGGTATTCGGGGGCGGTACTAAGCTGACAGTC
```

### Example 4. Addition of Linkers to the Hinge Region

The invention can also include different linker lengths and compositions, including Gly-Gly-Ser repeats (GGS)ₓ and (Gly-Gly-Gly-Gly-Ser)_{X} (SEQ ID NO:230) repeats GGGGSₓ and possibly other permutations. Other linkers include alone or in combination: GGS, GP, GPGP (SEQ ID NO:231), or GGSGGS (SEQ ID NO:232), which can wherein x=1, 2, 3, 4, 5, 6, 7, 8, 9, or 10×. In addition to binding and neutralization studies, the expression level and stability of each construct can be determined to improve antibody design.

Variable region (Fab, will be different for each mAb)-Constant region (Fab, CH1 domain)-Linker-Constant region (Fc, CH2 and CH3 domains).

Variable region (Fab, will be different for each mAb)-Constant region (Fab, CH1 domain)-Hinge region between CH1 and CH2 domains-Constant region (Fc, CH2 and CH3 domains).

CH1-CH3 domains are in the constant region of human IgG1 and do not change between different antibodies.

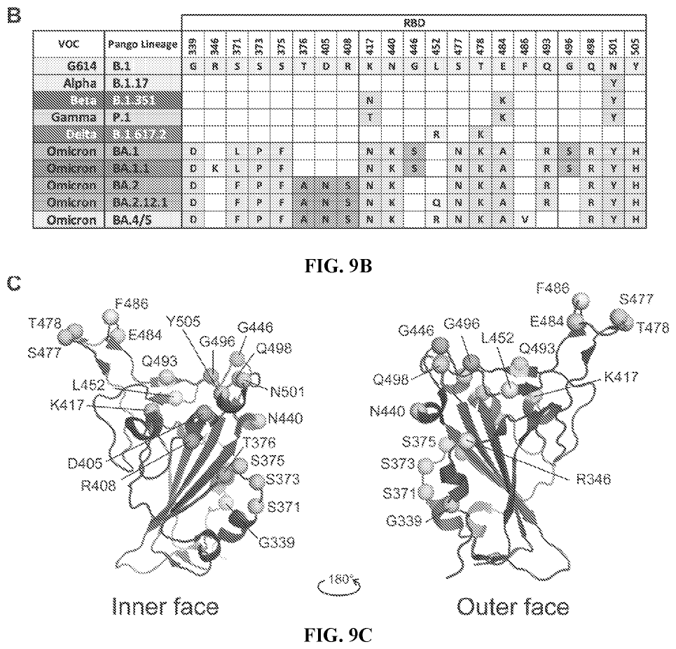

FIGS. 9A to 9C. Variant-related mutations on the SARS-CoV-2 Spike. (FIG. 9A) Spike primary structure with subunit domain boundaries denoted. SP: signal peptide; NTD: N-terminal domain; RBD: receptor-binding domain; SD1: subdomain 1; SD2: subdomain 2; S1/S2: furin cleavage site; S2′: S2 sub-cleavage site; FP: fusion peptide; CH: central helix; HR1: heptad repeat 1; CD: connector domain; HR2: heptad repeat 2; TM: transmembrane domain; CT: cytoplasmic tail. (FIG. 9B) Positions and substitutions of variant-related mutations located in the RBD, compared to the B.1 lineage. The World Health Organization variant designation and Pango lineage are indicated for each variant of concern (VOC). (FIG. 9C) Cartoon representation of the RBD and positions of variant-related mutations. Mutations shared among Omicron variants, but not with other VOCs are shaded accordingly. Those specific to BA.1 and BA.2 are shaded respectively. Mutations found in multiple VOCs are shaded throughout, as well, including N501Y. Positions that are less frequently mutated in multiple VOCs (e.g., L452 and R346) are shaded as well.

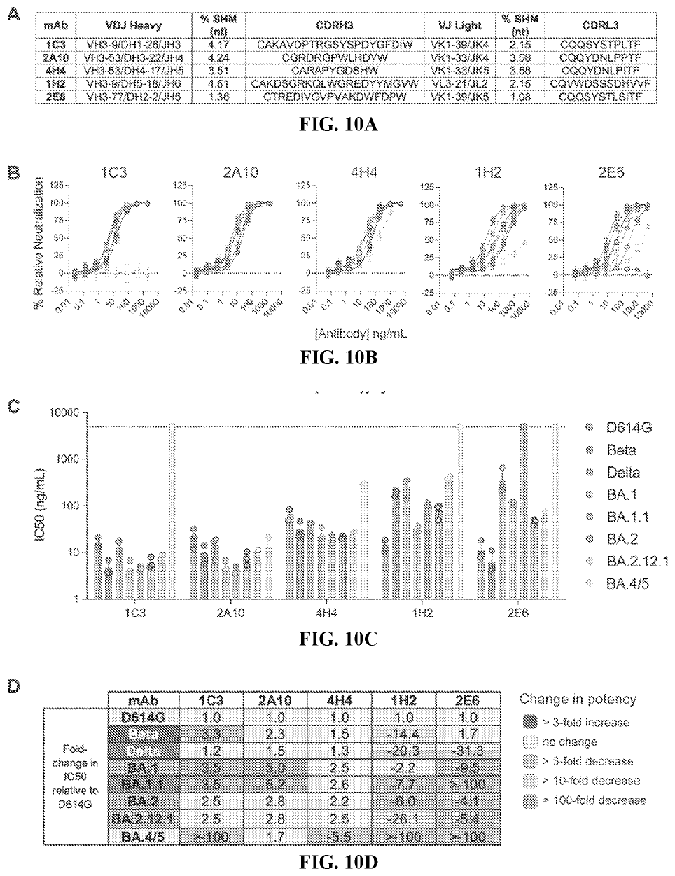

FIGS. 10A to 10D. Broadly neutralizing antibodies from a vaccinee. (FIG. 10A) The heavy (top) and light (bottom) germline, percent somatic hypermutation (SHM) and CDR3 sequence is shown for each bnAb. Middle column SEQ ID NOS: 233, 234, 235, 236, 237, and 238, right column SEQ ID NOS: 239, 240, 241, 242, 243, and 244, respectively. (FIG. 10B) Neutralization of SARS-CoV-2 pseudoviruses bearing the spike of the indicated VOC. Color coding corresponds to the key shown in (C). (FIG. 10C) IC₅₀ values for each neutralization curve in (B), calculated by nonlinear regression analysis. The limit of detection (L.O.D.) is shown as a dotted line and indicates the maximum antibody concentration used in the experiment. Error bars for panels B and C indicate the standard deviation from the mean for at least three independent experiments, each performed in technical duplicates. (FIG. 10D) Fold-change in IC50 for each antibody:variant pair relative to D614G.

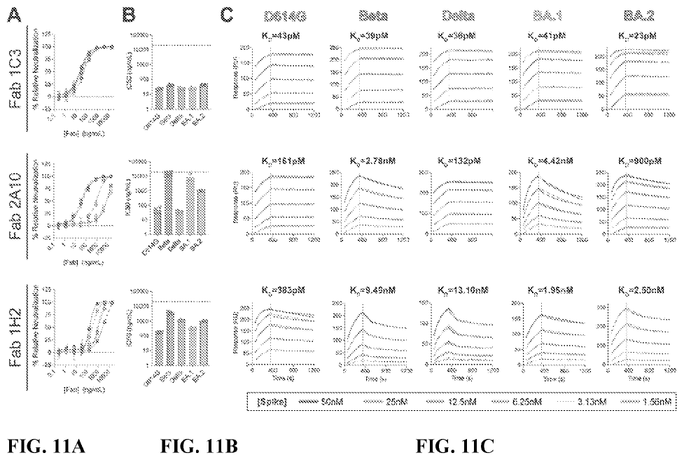

FIGS. 11A to 11C. Neutralization and kinetic analysis of select variants of concern and Fab fragments. (FIG. 11A) Neutralization of SARS-CoV-2 pseudoviruses bearing the spike of the indicated VOC by the Fab of each indicated antibody. (FIG. 11B) IC₅₀ values for each neutralization curve, calculated by nonlinear regression analysis. The limit of detection (L.O.D.), shown as a dotted line, indicates the maximum concentration of antibody used in the experiment (5000 ng/mL). For panels A and B, error bars indicate the standard deviation from the mean of four experiments, each performed in technical duplicates. (FIG. 11C) SPR analysis of each Fab:Spike pair, as indicated. The Fab ligand used for each experiment remains constant across each row and the spike analyte remains constant for each column. The average K_{D} obtained from eight replicate experiments is shown above each plot. Raw data are colored gray, with the 1:1 fit colored according to the spike concentration. For 1H2, the dissociation was truncated to 450 secs to capture an accurate off rate.

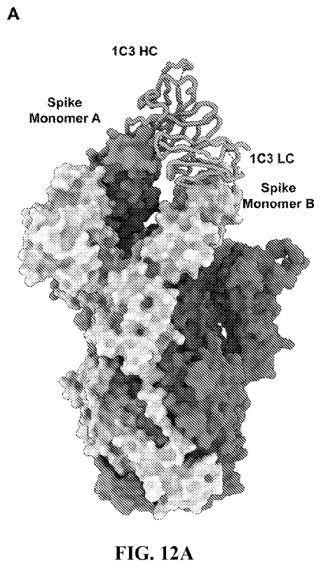

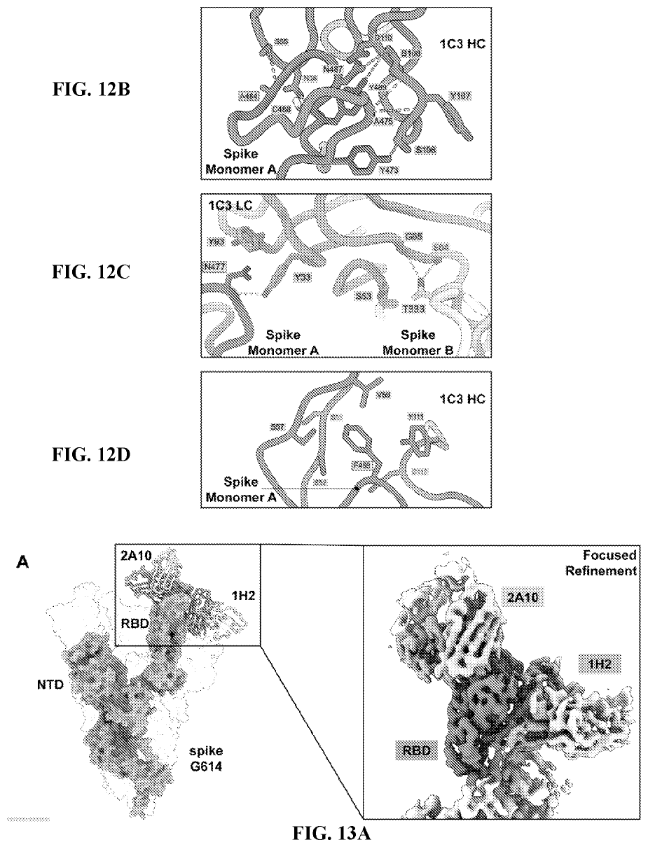

FIGS. 12A to 12D. Cryo-EM analysis of the 1C3-spike complex and interactions between 1C3 and Omicron BA.1 RBD. (FIG. 12A) Model of cryogenic electron microscopy structure of 1C3 antigen-binding fragment (Fab) in complex with SARS-CoV-2 Omicron BA.1 spike (PDB: 8F0G). One copy of 1C3 Fab (shown as cartoon representation with the HC and LC shown in dark and light, respectively) simultaneously contacts two spike monomers (shown as a surface representation), A (gray), and B (light gray). (B) SARS-CoV-2 Omicron spike interactions with 1C3, a closeup view of 1C3 HC interacting with the spike RBD in monomer A. Dashed lines indicate hydrogen bonds. (FIG. 12C) Closeup view of 1C3 light chain interacting with spike RBD in monomers A and B. Dashed lines indicate hydrogen bonds. (FIG. 12D) RBD residue F486 in BA.1, which is mutated to Valine in BA.4/5, forms intensive hydrophobic contacts with 1C3 HC residues.

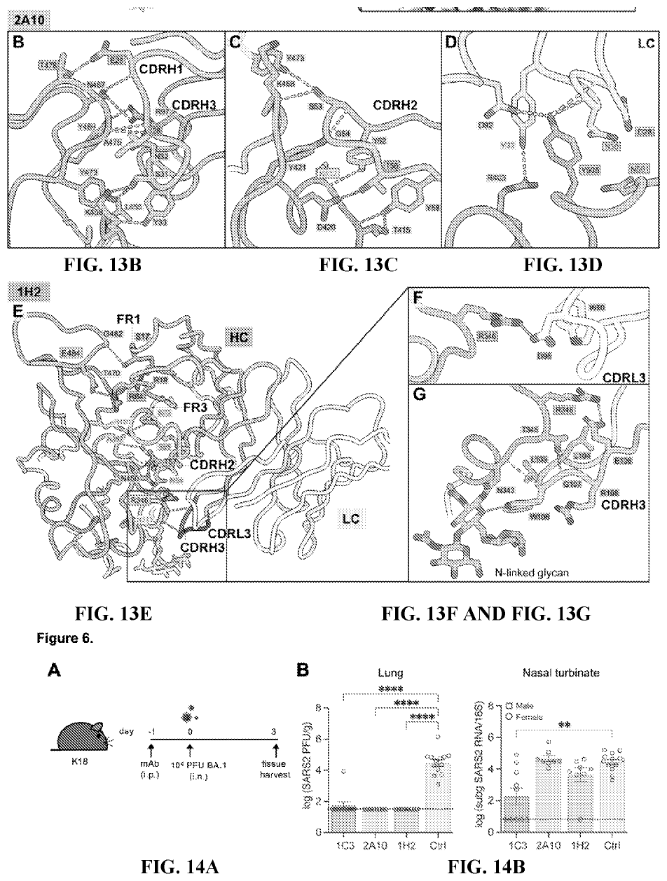

FIGS. 13A to 13G. Cryo-EM analysis of 2A10-1H2-spike complex and interactions between 2A10, 1H2 and D614G RBD. (FIG. 13A) Model of cryogenic electron microscopy structure of 1H2 and 2A10 antigen-binding fragments (Fab) in complex with SARS-CoV-2 D614G spike. The local refined structure of the RBD complexed with the two Fabs is modeled on the D614G spike model (shown as surface representation, PDB: 8F0H). Only one copy of each of the two Fabs is shown as a cartoon representation. 2A10 is shaded darker (HC) and lighter (LC), and 1H2 is also provided in dark (HC), and light (LC). (FIG. 13B-FIG. 13D) Interactions between 2A10 and D614G spike. 2A10 mainly utilizes heavy chain complementarity-determining region (CDR) H1 (B) and CDRH2 (C) and LC residues from CDR L2 and L3 (D) to contact the spike. Hydrogen bonds are indicated by dashed lines. Outlines highlight 2A10 residues that are mutated from germline as well as spike residues mutated in Omicron variants (K417N, T478K, N501Y, and Y505H). (FIG. 13E-FIG. 13G) Interactions between 1H2 and D614G spike. Contacts to the spike RBD by 1H2 residues from framework region (FR) 1, CDRL2, and FR3 are shown in (E). Zoomed-in views of contacts are shown in (F) for CDRL3 and in (G) for CDRH3. N343-linked glycans are shown as sticks. Hydrogen bonds are indicated by the dashed lines. 1H2 residue R84 is mutated from the germline and is outlined. Spike residues mutated in Omicron variants (R346K in BA.1.1, E484A) are also labeled with outlines.

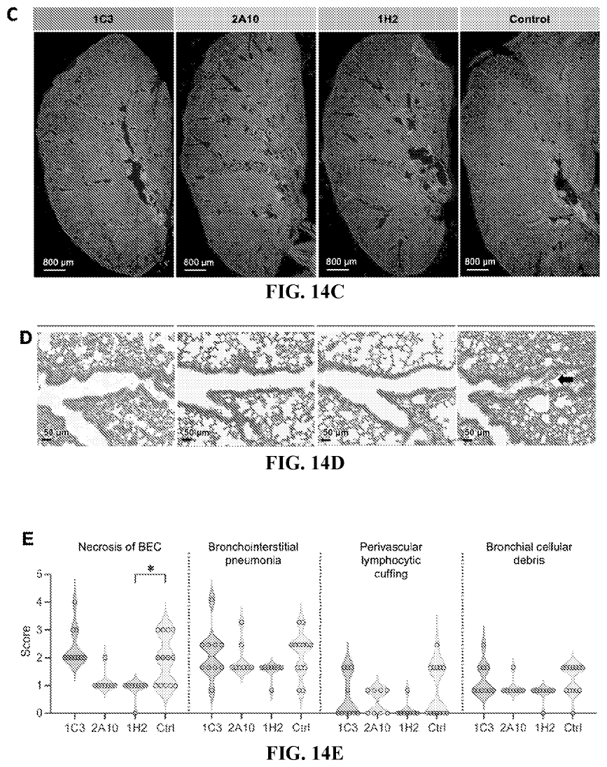

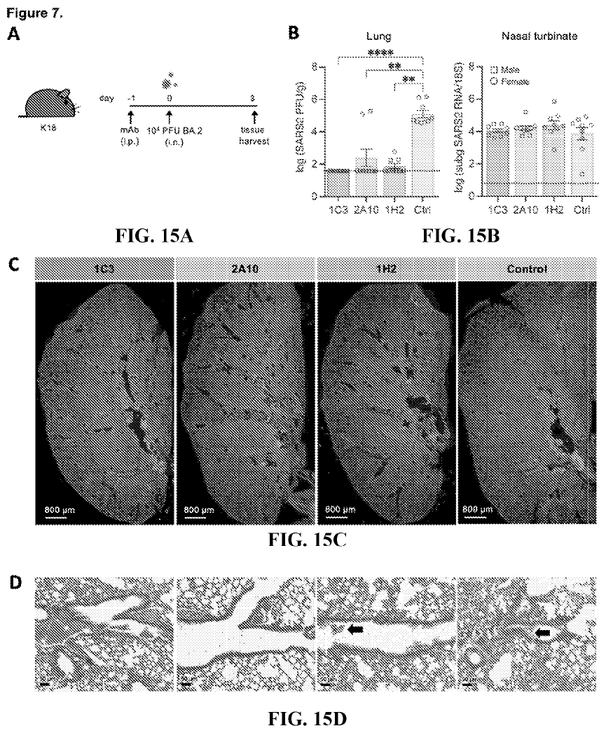

FIGS. 14A to 14E. Antibody-mediated protection of BA.1 challenge in K18-huACE2 mice. (14A) Experiment design for B to D. Mice received a single 10 mg/kg injection of 1C3, 2A10, 1H2 or control anti-SARS1 mAb intraperitoneally (i.p.) on day −1 and were challenged intranasally (i.n.) with 10⁴ PFU of SARS CoV-2 BA.1 one day later (day 0). Tissues were harvested 3 days post-infection (dpi). (FIG. 14B) Infectious virus titers measured by plaque assay of lungs collected from mice challenged with 104 PFU SARS CoV-2 BA.1 after passive transfer with mAb. Subgenomic 7a SARS CoV-2 RNA detected by RT-qPCR in nasal turbinate 3 dpi. (FIG. 14C) Lung images are representative of the distribution of immunofluorescence SARS CoV-2 N protein staining (magenta). (FIG. 14D) Representative histopathology images from lung samples stained with H&E. An arrow indicates bronchiolar epithelial cell necrosis. (FIG. 14E) Histopathological features of SARS CoV-2 infection in the lungs of mice were scored from 0 (least severe) to 5 (most severe). For panels B and E, data represent two independent experiments for a total of n=8-13 mice per group. Individual mice are represented as a square (male mouse) or circle (female mouse). Error bars indicate the SEM. Statistical analysis by Kruskal-Wallis test. *P<0.05, **P<0.01, and ***P<0.001. Only significant differences are shown. The limit of detection of each assay is represented by a dotted line.

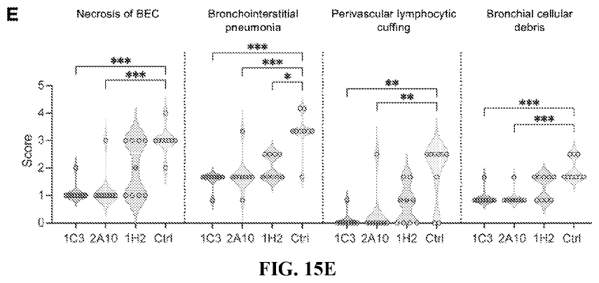

FIGS. 15A to 15E. Antibody-mediated protection of BA.2 challenge in K18-huACE2 mice. (FIG. 15A) Experiment design for panels B to D. 4 groups of mice were injected via intraperitoneal injection (i.p.) with 10 mg/kg 1C3, 2A10, or 1H2, or 200 ul PBS on day −1. Mice were dosed intranasally (i.n.) with 10⁴ PFU of SARS CoV-2 BA.2 at day 0 and organs were harvested 3 days post-infection (dpi). (FIG. 15B) Infectious viral particles in lungs were titrated using plaque assay. Replicating virus detected by RT-qPCR of subgenomic 7a SARS CoV-2 RNA in nasal turbinate 3 dpi. (FIG. 15C) Immunofluorescent distribution of SARS CoV-2 N protein staining (magenta) of lungs. (FIG. 15D) Representative histopathology images from lung samples stained with H&E. Arrows indicate bronchiolar epithelial cell necrosis. (FIG. 15E) Histopathological features of SARS CoV-2 infection in the lungs of mice were scored from 0 (least severe) to 5 (most severe). Data for panels B and E represent two independent experiments for a total of n=9 mice per group, with each mouse represented as a square (male mouse) or circle (female mouse). Error bars indicate the SEM. Statistical analysis by Kruskal-Wallis test. *P<0.05, **P<0.01, and ***P<0.001. Only significant differences are indicated. Dotted line represents the limit of detection of each assay.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

1. A monoclonal antibody or antigen-binding fragment thereof comprising heavy chain complementarity determining regions (CDR) H1, CDRH2, and CDRH3, and light chain CDRL1, CDRL2, and CDRL3 of:
SEQ ID NOS: 1, 2, 3 and 6, 7, 8;
SEQ ID NOS: 11, 12, 13 and 16, 17, 18;
SEQ ID NOS: 21, 22, 23 and 36, 37, 38; or 26, 27, 28 and 36, 37, 38; or 31, 32, 33 and 36, 37, 38;
SEQ ID NOS: 41, 42, 43 and 51, 52, 53; or 46, 47, 48 and 51, 52, 53;
SEQ ID NOS: 56, 57, 58 and 61, 62, 63;
SEQ ID NOS: 66, 67, 68 and 71, 72, 73;
SEQ ID NOS: 76, 77, 78 and 81, 82, 83;
SEQ ID NOS: 86, 87, 88 and 91, 92, 93;
SEQ ID NOS: 96, 97, 98 and 106, 107, 108; or 101, 102, 103 and 106, 107, 108;
SEQ ID NOS: 111, 112, 113 and 116, 117, 118;
SEQ ID NOS: 121, 122, 123 and 126, 127, 128;
SEQ ID NOS: 131, 132, 133 and 136, 137, 138;
SEQ ID NOS: 141, 142, 143 and 151, 152, 153; or 146, 147, 148 and 151, 152, 153;
SEQ ID NOS: 156, 157, 158 and 161, 162, 163;
SEQ ID NOS: 166, 167, 168 and 171, 172, 173;
SEQ ID NOS: 176, 177, 178 and 181, 182, 183; or 176, 177, 178 and 186, 187, 188;
SEQ ID NOS: 191, 192, 193 and 196, 197, and 198;
SEQ ID NOS: 201, 202, 203 or 204, 205, 206 and 207, 206, 208 or 207, 206, 210; or
SEQ ID NOS: 219, 220, 221, and 222, 223, and 224, respectively.

2. The monoclonal antibody of claim 1, wherein the antibody or antigen-binding fragment is cross-reactive to and binds to one or more, or at least two, variants of a Spike protein of SARS-CoV-2 (SARS2-S), and wherein the antibody is a neutralizing antibody.

3. The monoclonal antibody or antigen-binding fragment thereof of claim 1, wherein the antibody heavy chain variable region and light chain variable region comprises an amino acid sequence at least about 90, 91, 92, 93, 94, 95, 96, 97, 98, 99, or 100% identical to the amino acid sequence of SEQ ID NOS: 4 and 9; 14 and 19; 24 and 39, or 29 and 39, or 34 and 39; 44 and 54, or 49 and 54; 59 and 64; 69 and 74; 79 and 84; 89 and 94; 99 and 109, or 104 and 109; 114 and 119; 124 and 129; 134 and 139; 144 and 154, or 149 and 154; 159 and 164; 169 and 174; 179 and 184, or 179 and 189; 194 and 199, 211 and 213, 215 and 217, 225 and 226, or 225 and 227, respectively; or
the antibody heavy chain variable region and light chain variable region is encoded by a nucleic acid sequence having at least 90, 91, 92, 93, 94, 95, 96, 97, 98, 99, or 100% identical to a nucleic acid of SEQ ID NOS: 5 and 10; 15 and 20; 25 and 40, or 30 or 40, or 35 or 40; 45 and 55, or 46 and 55; 60 and 65; 70 and 75; 80 and 85; 90 and 95; 100 and 110 or 105 and 110; 115 and 120; 125 and 130; 135 and 140; 145 and 155, or 150 and 155; 160 and 165; 170 and 175;
180 and 185, or 180 and 190; 195 and 200, 212 and 214, 216 and 218, 226 and 229, respectively.

4. The monoclonal antibody or antigen-binding fragment thereof of claim 1, wherein at least one of:
the antigen-binding fragment is a recombinant single-chain fragment variable (scFV) antibody, Fab fragment, F(ab′)2 fragment, or Fv fragment;
the antibody or antigen-binding fragment is chimeric, humanized, fully human, or bispecific;
the antibody or antigen-binding fragment comprises an Fc portion mutated to at least one of: eliminate or enhance Fc Receptor (FcR) interactions to change a half-life, increase or decrease antibody-dependent cellular cytotoxicity, or increase or decrease complement activation;
the antibody or antigen-binding fragment is adapted for administration or genetic delivery with an RNA or DNA sequence or vector encoding the antibody or antigen-binding fragment; or
the antibody or antigen-binding fragment further comprises a peptide linker in a hinge region of the heavy chain.

5.-9. (canceled)

10. The monoclonal antibody or antigen-binding fragment thereof of claim 1, further comprising a pharmaceutical composition comprising a pharmaceutically acceptable excipient and a therapeutically effective amount of a monoclonal antibody or antigen-binding fragment thereof.

11.-19. (canceled)

20. A method of treating SARS-CoV-2 in a subject in need thereof, the method comprising administering to the subject a monoclonal antibody or antigen-binding fragment thereof comprising heavy chain complementarity determining regions (CDR) H1, CDRH2, and CDRH3, and light chain CDRL1, CDRL2, and CDRL3 of:
SEQ ID NOS: 1, 2, 3 and 6, 7, 8;
SEQ ID NOS: 11, 12, 13 and 16, 17, 18;
SEQ ID NOS: 21, 22, 23 and 36, 37, 38; or 26, 27, 28 and 36, 37, 38; or 31, 32, 33 and 36, 37, 38;
SEQ ID NOS: 41, 42, 43 and 51, 52, 53; or 46, 47, 48 and 51, 52, 53;
SEQ ID NOS: 56, 57, 58 and 61, 62, 63;
SEQ ID NOS: 66, 67, 68 and 71, 72, 73;
SEQ ID NOS: 76, 77, 78 and 81, 82, 83;
SEQ ID NOS: 86, 87, 88 and 91, 92, 93;
SEQ ID NOS: 96, 97, 98 and 106, 107, 108; or 101, 102, 103 and 106, 107, 108;
SEQ ID NOS: 111, 112, 113 and 116, 117, 118;
SEQ ID NOS: 121, 122, 123 and 126, 127, 128;
SEQ ID NOS: 131, 132, 133 and 136, 137, 138;
SEQ ID NOS: 141, 142, 143 and 151, 152, 153; or 146, 147, 148 and 151, 152, 153;
SEQ ID NOS: 156, 157, 158 and 161, 162, 163;
SEQ ID NOS: 166, 167, 168 and 171, 172, 173;
SEQ ID NOS: 176, 177, 178 and 181, 182, 183; or 176, 177, 178 and 186, 187, 188;
SEQ ID NOS: 191, 192, 193 and 196, 197, and 198;
SEQ ID NOS: 201, 202, 203 or 204, 205, 206 and 207, 206, 208 or 207, 206, 210; or
SEQ ID NOS: 219, 220, 221, and 222, 223, and 224, respectively.

21. The method of claim 20, wherein the antibody or antigen-binding fragment is cross-reactive to one or more, or at least two, variants of a Spike protein of SARS-CoV-2 (SARS2-S), and wherein the antibody is a neutralizing antibody.

22. The method of claim 20, wherein the antibody heavy chain variable region and light chain variable region comprises an amino acid sequence at least about 90, 91, 92, 93, 94, 95, 96, 97, 98, 99, or 100% identical to the amino acid sequence of SEQ ID NOS: 4 and 9; 14 and 19; 24 and 39, or 29 and 39, or 34 and 39; 44 and 54, or 49 and 54; 59 and 64; 69 and 74; 79 and 84; 89 and 94; 99 and 109, or 104 and 109; 114 and 119; 124 and 129; 134 and 139; 144 and 154, or 149 and 154; 159 and 164; 169 and 174; 179 and 184, or 179 and 189; 194 and 199,211 and 213, 215 and 217, 225 and 226, or 225 and 227, respectively; or
the antibody heavy chain variable region and light chain variable region is encoded by a nucleic acid sequence having at least 90, 91, 92, 93, 94, 95, 96, 97, 98, 99, or 100% identical to a nucleic acid of SEQ ID NOS: 5 and 10; 15 and 20; 25 and 40, or 30 or 40, or 35 or 40; 45 and 55, or 46 and 55; 60 and 65; 70 and 75; 80 and 85; 90 and 95; 100 and 110 or 105 and 110; 115 and 120; 125 and 130; 135 and 140; 145 and 155, or 150 and 155; 160 and 165; 170 and 175; 180 and 185, or 180 and 190; 195 and 200, 212 and 214, 216 and 218, 226 and 229, respectively.

23. The method of claim 20, wherein at least one of:
the antigen-binding fragment is a recombinant single-chain fragment variable (scFV) antibody, Fab fragment, F(ab′)2 fragment, or Fv fragment;
the antibody or antigen-binding fragment is chimeric, humanized, fully human, or bispecific;
the antibody or antigen-binding fragment comprises an Fc portion mutated to at least one of: eliminate or enhance Fc Receptor (FcR) interactions to change a half-life, increase or decrease antibody-dependent cellular cytotoxicity, or increase or decrease complement activation;
the antibody or antigen-binding fragment is adapted for administration or genetic delivery with an RNA or DNA sequence or vector encoding the antibody or antigen-binding fragment; or
the antibody or antigen-binding fragment further comprises a peptide linker in a hinge region of the heavy chain.

24.-27. (canceled)

28. A pharmaceutical device suitable for nasal or pulmonary delivery of monoclonal antibody or antigen-binding fragment thereof comprising heavy chain complementarity determining regions (CDR) H1, CDRH2, and CDRH3, and light chain CDRL1, CDRL2, and CDRL3 of:
SEQ ID NOS: 1, 2, 3 and 6, 7, 8;
SEQ ID NOS: 11, 12, 13 and 16, 17, 18;
SEQ ID NOS: 21, 22, 23 and 36, 37, 38; or 26, 27, 28 and 36, 37, 38; or 31, 32, 33 and 36, 37, 38;
SEQ ID NOS: 41, 42, 43 and 51, 52, 53; or 46, 47, 48 and 51, 52, 53;
SEQ ID NOS: 56, 57, 58 and 61, 62, 63;
SEQ ID NOS: 66, 67, 68 and 71, 72, 73;
SEQ ID NOS: 76, 77, 78 and 81, 82, 83;
SEQ ID NOS: 86, 87, 88 and 91, 92, 93;
SEQ ID NOS: 96, 97, 98 and 106, 107, 108; or 101, 102, 103 and 106, 107, 108;
SEQ ID NOS: 111, 112, 113 and 116, 117, 118;
SEQ ID NOS: 121, 122, 123 and 126, 127, 128;
SEQ ID NOS: 131, 132, 133 and 136, 137, 138;
SEQ ID NOS: 141, 142, 143 and 151, 152, 153; or 146, 147, 148 and 151, 152, 153;
SEQ ID NOS: 156, 157, 158 and 161, 162, 163;
SEQ ID NOS: 166, 167, 168 and 171, 172, 173;
SEQ ID NOS: 176, 177, 178 and 181, 182, 183; or 176, 177, 178 and 186, 187, 188;
SEQ ID NOS: 191, 192, 193 and 196, 197, and 198;
SEQ ID NOS: 201, 202, 203 or 204, 205, 206 and 207, 206, 208 or 207, 206, 210; or
SEQ ID NOS: 219, 220, 221, and 222, 223, and 224,
respectively, selected from an inhaler for liquids, a nebulizer, metered-dose inhaler, aerosol, and a dry powder inhaler.

29. The pharmaceutical device of claim 28, wherein the antibody or antigen-binding fragment is cross-reactive to one or more, or at least two, variants of a Spike protein of SARS-CoV-2 (SARS2-S).

30. A method for detecting a variant of a Spike protein of SARS-CoV-2 (SARS2-S) comprising:
obtaining or having obtained a biological sample suspected of comprising a SARS-CoV-2 virus;
contacting the biological sample with a monoclonal antibody or antigen-binding fragment thereof comprising heavy chain complementarity determining regions (CDR) H1, CDRH2, and CDRH3, and light chain CDRL1, CDRL2, and CDRL3 of:
SEQ ID NOS: 1, 2, 3 and 6, 7, 8;
SEQ ID NOS: 11, 12, 13 and 16, 17, 18;
SEQ ID NOS: 21, 22, 23 and 36, 37, 38; or 26, 27, 28 and 36, 37, 38; or 31, 32, 33 and 36, 37, 38;
SEQ ID NOS: 41, 42, 43 and 51, 52, 53; or 46, 47, 48 and 51, 52, 53;
SEQ ID NOS: 56, 57, 58 and 61, 62, 63;
SEQ ID NOS: 66, 67, 68 and 71, 72, 73;
SEQ ID NOS: 76, 77, 78 and 81, 82, 83;
SEQ ID NOS: 86, 87, 88 and 91, 92, 93;
SEQ ID NOS: 96, 97, 98 and 106, 107, 108; or 101, 102, 103 and 106, 107, 108;
SEQ ID NOS: 111, 112, 113 and 116, 117, 118;
SEQ ID NOS: 121, 122, 123 and 126, 127, 128;
SEQ ID NOS: 131, 132, 133 and 136, 137, 138;
SEQ ID NOS: 141, 142, 143 and 151, 152, 153; or 146, 147, 148 and 151, 152, 153;
SEQ ID NOS: 156, 157, 158 and 161, 162, 163;
SEQ ID NOS: 166, 167, 168 and 171, 172, 173;
SEQ ID NOS: 176, 177, 178 and 181, 182, 183; or 176, 177, 178 and 186, 187, 188;
SEQ ID NOS: 191, 192, 193 and 196, 197, and 198;
SEQ ID NOS: 201, 202, 203 or 204, 205, 206 and 207, 206, 208 or 207, 206, 210; or
SEQ ID NOS: 219, 220, 221, and 222, 223, and 224, respectively; and
detecting the binding of the antibody or antigen-binding fragment to the SARS-CoV-2 virus.

31. The method of claim 30, wherein the step of detecting the antibody or antigen-binding fragment is diagnostic for the detection of SARS-CoV-2 in the biological sample.

32. The method of claim 30, further comprising detecting SARS-CoV-2 by performing an immunoassay on the biological sample from a subject; wherein the immunoassay uses an antibody or antigen-binding fragment, wherein the immunoassay selected from radioimmunoassay, enzyme-linked immunosorbent assay (ELISA), sandwich assays, Western blot, immunoprecipitation, immunohistochemistry, immunofluorescence, antibody microarray, dot blotting, and fluorescence-activated cell sorting (FACS).

33. (canceled)

34. A kit for detection of one or more variants of a Spike protein of SARS-CoV-2 comprising a monoclonal antibody or antigen-binding fragment thereof comprising heavy chain complementarity determining regions (CDR) H1, CDRH2, and CDRH3, and light chain CDRL1, CDRL2, and CDRL3 of:
SEQ ID NOS: 1, 2, 3 and 6, 7, 8;
SEQ ID NOS: 11, 12, 13 and 16, 17, 18;
SEQ ID NOS: 21, 22, 23 and 36, 37, 38; or 26, 27, 28 and 36, 37, 38; or 31, 32, 33 and 36, 37, 38;
SEQ ID NOS: 41, 42, 43 and 51, 52, 53; or 46, 47, 48 and 51, 52, 53;
SEQ ID NOS: 56, 57, 58 and 61, 62, 63;
SEQ ID NOS: 66, 67, 68 and 71, 72, 73;
SEQ ID NOS: 76, 77, 78 and 81, 82, 83;
SEQ ID NOS: 86, 87, 88 and 91, 92, 93;
SEQ ID NOS: 96, 97, 98 and 106, 107, 108; or 101, 102, 103 and 106, 107, 108;
SEQ ID NOS: 111, 112, 113 and 116, 117, 118;
SEQ ID NOS: 121, 122, 123 and 126, 127, 128;
SEQ ID NOS: 131, 132, 133 and 136, 137, 138;
SEQ ID NOS: 141, 142, 143 and 151, 152, 153; or 146, 147, 148 and 151, 152, 153;
SEQ ID NOS: 156, 157, 158 and 161, 162, 163;
SEQ ID NOS: 166, 167, 168 and 171, 172, 173;
SEQ ID NOS: 176, 177, 178 and 181, 182, 183; or 176, 177, 178 and 186, 187, 188;
SEQ ID NOS: 191, 192, 193 and 196, 197, and 198;
SEQ ID NOS: 201, 202, 203 or 204, 205, 206 and 207, 206, 208 or 207, 206, 210; or
SEQ ID NOS: 219, 220, 221, and 222, 223, and 224, respectively, for the isolation and/or detection of SARS-CoV-2 in one or more sample types of human or nonhuman origin, including: blood, plasma, serum, saliva, tears, cerebrospinal fluid, lymph, urine, feces, exhaled breath condensate, perspiration, amniotic fluid, exosomes, cell and tissue lysates.

35. A recombinant nucleic acid molecule encoding an antibody or antigen-binding fragment thereof comprising heavy chain complementarity determining regions (CDR) H1, CDRH2, and CDRH3, and light chain CDRL1, CDRL2, and CDRL3 of:
SEQ ID NOS: 1, 2, 3 and 6, 7, 8;
SEQ ID NOS: 11, 12, 13 and 16, 17, 18;
SEQ ID NOS: 21, 22, 23 and 36, 37, 38; or 26, 27, 28 and 36, 37, 38; or 31, 32, 33 and 36, 37, 38;
SEQ ID NOS: 41, 42, 43 and 51, 52, 53; or 46, 47, 48 and 51, 52, 53;
SEQ ID NOS: 56, 57, 58 and 61, 62, 63;
SEQ ID NOS: 66, 67, 68 and 71, 72, 73;
SEQ ID NOS: 76, 77, 78 and 81, 82, 83;
SEQ ID NOS: 86, 87, 88 and 91, 92, 93;
SEQ ID NOS: 96, 97, 98 and 106, 107, 108: or 101, 102, 103 and 106, 107, 108;
SEQ ID NOS: 111, 112, 113 and 116, 117, 118;
SEQ ID NOS: 121, 122, 123 and 126, 127, 128;
SEQ ID NOS: 131, 132, 133 and 136, 137, 138;
SEQ ID NOS: 141, 142, 143 and 151, 152, 153; or 146, 147, 148 and 151, 152, 153;
SEQ ID NOS: 156, 157, 158 and 161, 162, 163;
SEQ ID NOS: 166, 167, 168 and 171, 172, 173;
SEQ ID NOS: 176, 177, 178 and 181, 182, 183: or 176, 177, 178 and 186, 187, 188;
SEQ ID NOS: 191, 192, 193 and 196, 197, and 198;
SEQ ID NOS: 201, 202, 203 or 204, 205, 206 and 207, 206, 208 or 207, 206, 210; or
SEQ ID NOS: 219, 220, 221, and 222, 223, and 224, respectively.

36. The recombinant nucleic acid molecule of claim 35, the nucleic acid molecule encoding the antigen-binding fragment thereof, wherein the antibody heavy chain variable region and light chain variable region is encoded by a nucleic acid sequence having at least 90, 91, 92, 93, 94, 95, 96, 97, 98, 99, or 100% identical to the nucleic acid of SEQ ID NOS: 5 and 10; 15 and 20; 25 and 40, or 30 or 40, or 35 or 40; 45 and 55, or 46 and 55; 60 and 65; 70 and 75; 80 and 85; 90 and 95; 100 and 110 or 105 and 110; 115 and 120; 125 and 130; 135 and 140; 145 and 155, or 150 and 155; 160 and 165; 170 and 175; 180 and 185, or 180 and 190; 195 and 200; 212 and 214; 216 and 218; or 226 and 229, respectively.

37. The recombinant nucleic acid molecule of claim 35, further comprising an expression control sequence operatively linked to the recombinant nucleic acid molecule.

38. The recombinant nucleic acid molecule of claim 37, wherein the vector is in a host cell.

39. A method of making an antibody or antigen-binding fragment of an antibody comprising culturing a recombinant host cell comprising a recombinant expression construct comprising an expression control sequence operatively linked to a recombinant nucleic acid molecule encoding a monoclonal antibody or binding fragment thereof comprising heavy chain complementarity determining regions (CDR) H1, CDRH2, and CDRH3, and light chain CDRL1, CDRL2, and CDRL3 of:
SEQ ID NOS: 1, 2, 3 and 6, 7, 8;
SEQ ID NOS: 11, 12, 13 and 16, 17, 18;
SEQ ID NOS: 21, 22, 23 and 36, 37, 38; or 26, 27, 28 and 36, 37, 38; or 31, 32, 33 and 36, 37, 38;
SEQ ID NOS: 41, 42, 43 and 51, 52, 53; or 46, 47, 48 and 51, 52, 53;
SEQ ID NOS: 56, 57, 58 and 61, 62, 63;
SEQ ID NOS: 66, 67, 68 and 71, 72, 73;
SEQ ID NOS: 76, 77, 78 and 81, 82, 83;
SEQ ID NOS: 86, 87, 88 and 91, 92, 93;
SEQ ID NOS: 96, 97, 98 and 106, 107, 108; or 101, 102, 103 and 106, 107, 108;
SEQ ID NOS: 111, 112, 113 and 116, 117, 118;
SEQ ID NOS: 121, 122, 123 and 126, 127, 128;
SEQ ID NOS: 131, 132, 133 and 136, 137, 138;
SEQ ID NOS: 141, 142, 143 and 151, 152, 153; or 146, 147, 148 and 151, 152, 153;
SEQ ID NOS: 156, 157, 158 and 161, 162, 163;
SEQ ID NOS: 166, 167, 168 and 171, 172, 173;
SEQ ID NOS: 176, 177, 178 and 181, 182, 183; or 176, 177, 178 and 186, 187, 188;
SEQ ID NOS: 191, 192, 193 and 196, 197, and 198;
SEQ ID NOS: 201, 202, 203 or 204, 205, 206 and 207, 206, 208 or 207, 206, 210; or
SEQ ID NOS: 219, 220, 221, and 222, 223, and 224, respectively, wherein the host cell produces the antibody or antigen-binding fragment of an antibody, and isolating the antibody or antigen-binding fragment of an antibody from the host cell.
